(19) United States
(12) Patent Application Publication
(10) Pub. No.: US 2011/0196189 A1
(43) Pub. Date: Aug. 11, 2011

(54) EXTRA-CARDIAC DIFFERENTIAL VENTRICULAR ACTUATION BY INERTIAL AND BARIC PARTITIONING

(75) Inventor: Michael Milbocker, Holliston, MA (US)

(73) Assignee: MyoCardioCare, Inc., Fort Collins, CO (US)

(21) Appl. No.: 12/702,788

(22) Filed: Feb. 9, 2010

(51) Int. Cl. A61M 1/12

(52) U.S. Cl. 600/16

(57) ABSTRACT

A device for extra-cardiac actuation for the support of a failing heart is provided that utilizes the inertial component of a driving fluid to provide separately adjustable quantities of supportive energy to the left and right ventricles. By partitioning the inertial and baric forms of energy delivered to a single chamber, heart encircling assist device, and by localizing the heart to minimize lateral and axial displacement a device and method for achieving balanced left-right flow is disclosed that restores the Frank-Starling mechanism to a failing heart and assists recovery by enabling the heart to achieve physiologic cardiovascular equilibrium.

### BACKGROUND

An alternative to blood contacting devices for augmenting blood circulation in the body is known as extra-cardiac actuation, where force is applied to the epicardial surfaces of the heart's ventricles in order to increase one or more of the following: ejection fraction, cardiac output, stroke volume, arterial pulsatility, and mean arterial pressure.

Traditional approaches to extra-cardiac actuation have focused on delivering supplement pressure to the external surfaces of the ventricles during systole and relying on atrial pressure to refill the ventricles once the supplemental pressure is removed. Less common are extra-cardiac assist devices which supply both positive systolic pressure and negative (relative to chest cavity pressure) diastolic pressure to assist in the refilling of the ventricles with blood.

These non-blood contacting devices, similar in form but not function to the current invention, provide inadequate augmentation of cardiac function. The inadequacy is due to the form and timing of energy delivered to the heart. The heart employs three principle forms of energy; potential, inertial, and baric in a temporally distinct sequence.

Potential energy is stored in the heart walls. This mode of energy storage is frequently removed when the ventricular walls of the heart are constrained by an extra-cardiac assist device, and storage capacity is compromised in most failing hearts.

The heart develops inertial energy by rapidly increasing the velocity of blood in the major arteries. This form of energy cannot develop in the circulation if energy is too slowly delivered to the ventricles.

Baric or pressure energy is the usual form of energy delivery by extra-cardiac assist devices. However, pressure can be developed inside the ventricles in two ways. Extra-cardiac assist devices develop a transient pressure gradient in the walls of the ventricles assuming that an incremental increase in pressure (ΔP) at the surface translates to a ΔP increase in the ventricular blood volume. However, the heart develops pressure in two ways, neither of which employs a change in pressure at the epicardial surface. Tension develops in the heart wall that translates as a radial pressure, this tension is either compromised or absent in the extra-cardiac actuation assisted heart. Secondly, the rate at which the ventricular volume changes induces an accelerative term in the blood flow that is manifest as a pressure.

This accelerative pressure developed within the blood volume is needed to alter the proximal vascular structure to accept a bolus of blood volume. In this way the aorta acts as a secondary ventricle, storing potential energy during the initial systolic phase, and depositing this energy into the blood flow once the ventricular valves close during the end systolic phase. The native heart utilizes all three forms (potential, inertial and baric) of energy in a particular sequence to achieve physiologic blood flow. Traditional extra-cardiac assist devices fail to reproduce the timing and magnitude of each of these component energy forms.

A number of extra-cardiac assist devices have been described and studied that share the feature of compressing the outer epicardial surface, thereby generating a pressure gradient in the heart wall. These methods have focused on improving cardiac performance by assisting the systolic (positive pressure) function of the heart. Primarily systolic extra-cardiac actuation methods have been investigated only in the laboratory setting.

Examples of primary systolic extra-cardiac actuation techniques include, but are not limited to, cardiomyoplasty (the technique of wrapping skeletal muscle around the heart and artificially stimulating it), the Cardio support system (Cardio Technologies, Inc., Pinebrook, N.J.) and the “Heart Booster” (Abiomed, Inc., Danvers, Mass.).

Cumulative results from laboratory investigations using these devices have all resulted in similar findings. Specifically, primary systolic extra-cardiac actuation has been shown to enhance left ventricular (LV) systolic function without apparent change in native LV oxygen consumption requirements; suggesting this increase does not require extra work from the heart.

Primary systolic extra-cardiac assist devices have been shown to only benefit hearts with substantial degrees of LV failure. These hearts are typically congestive, presenting an abnormally large ventricular volume ideally suited for baric energy transfer. Primarily systolic extra-cardiac actuation techniques are associated with negative effects on diastolic right ventricle (RV) and left ventricle (LV) function.

This is exhibited by reductions in diastolic volume that, in part, explains the diminishing support potential on hearts in lesser degrees of failure (less dilated). Primary systolic extra-cardiac actuation relies on pathologically elevated ventricular filling pressures to achieve adequate diastolic ventricular volume. Thus, primary systolic extra-cardiac actuation tends to perpetuate heart failure conditions such as elevated atrial pressure, elevated pulmonary pressure, large ventricular volume, and reduced arterial pressure.

For these reasons, the timing of systolic support and the addition of diastolic support to the extra-cardiac actuation approach is needed if such devices are to support physiological hemodynamics. The duration of systolic support (positive pressure) relative to the duration of the cardiac cycle (systolic+diastolic) is of prime importance. Once the exiting valves of the ventricles close, it is critical that the ventricular blood pressure drop as rapidly as possible. The interval between valve closure and blood flow into the ventricle subtracts from the filling time (diastolic volume) or ejection time (systolic stroke volume).

In addition, the rate at which blood fills the ventricles is dependent upon the difference in ventricular blood pressure and the supplying atrial blood pressure. Primary systolic extra-cardiac assist devices have negative effects on the dynamics of diastolic relaxation and, in effect, reduce the rate of diastolic pressure decay (negative dP/dt max), increasing the time required for ventricular relaxation. This better explains why primary systolic extra-cardiac actuation techniques rely on substantial degrees of LV and RV loading (i.e., increased left and right atrial pressure or “preload”) to be effective.

The heart is comprised of four interconnected compliant blood reservoirs which interact dynamically. It is not only that the start and end volumes and pressures are important, but the more subtle aspects of timing, and the first and second time derivatives of pressure and volume as well as the partition of delivered energy into the associated potential, inertial and baric forms.

The following features may be found in a physiologic extra-cardiac assist device:
means to augment diastolic function of the heart
means to generate a physiologic left ventricular pressure-volume relationship (LVPVR)
means to generate a physiologic right ventricular pressure-volume relationship (RVPVR)
means to minimize abnormal septal motion, abnormal free wall motion (especially RV) axial heart expulsion from the device, and left-right heart volley within the device
means to minimize the load-dependence of performance by a more natural partition of delivered energy forms
means to minimize damage to the heart by minimizing energy misapplication by delivering supplemental energy in physiologically supportive forms
means to synchronize application of supplemental energy with native function of the heart
means to adjust the extra-cardiac assist device to a range of heart sizes
means to minimize trauma from shear forces and abrasion
means to provide improvement in end organ perfusion by increasing the inertial component of energy supplied to a patient's heart
means to extend the preservation time of donor organs intended for transplant, but increasing the inertial component of energy supplied to the heart
means to effectively support patients who have recently undergone bypass graft surgery
means to protect specific regions of the myocardium from forces applied by the action used to support the heart

The physiological extra-cardiac assist device is an example of one type of mechanical non-blood contacting cardiac assistance device that delivers supportive forms of energy to the heart.

In general, a physiological extra-cardiac assist device system comprises two primary elements: (a) a Cup having dynamic characteristics and material construction that keep the device's actuating liner or sheath closely conformed to the exterior surface (epicardium) of the heart throughout systolic and diastolic actuation, and (b) a Drive System serving as energy source and controller that cyclically applies hydraulic energy to the epicardium in a manner satisfying the above physiologic requirements.

Effective extra-cardiac actuation requires that the Cup and Drive System satisfy multiple and complex performance requirements. Preferred embodiments of the Cup of the present invention satisfy these critical performance requirements in a manner that is superior or lacking in prior art extra-cardiac assist devices.

Conventional extra-cardiac assist devices apply cyclic motion to the free walls of the heart to achieve a target volumetric displacement of blood during systole and in some devices volumetric filling of blood during diastole to the left and right ventricles.

Due to the interactive nature between left and right ventricles, and the differences in free wall compliances, ventricular pressures, filling pressures and ventricular shapes and the propensity for the heart to move within the device, simply changing the internal volume of the device does not achieve a physiologic volumetric change in the right and left ventricles. The abnormal volumetric changes that occur are caused by right ventricle left ventricle coupling. The coupling between right and left ventricles is increased by the presence of the extra-cardiac assist device, especially during systole.

Further complications include changes in total blood volume, left and right output resistance, vascular compliance, left and right atrial pressure, pulmonary resistance, and changes in blood chemistry. In addition there is the possibility of significant changes in load depending on the contractile state of the heart, ranging from a flaccid non-contractile heart to a contracted fibrillating heart. Interference can occur if the extra-cardiac actuation is not in synchrony with the native contractions, or the ratio of systolic to diastolic duration for device and heart is different, or the presence of premature ventricular contractions, either device induced or spontaneous.

Thus, the notion that repeatedly pushing inwardly on the exterior walls of the heart, and compressing the left and right ventricles into a systolic configuration, thereby improves hemodynamics, is rarely correct long term. There needs to be approximately equal volumes of blood expelled from the left and right ventricles, and the volumes must be sufficient to maintain normal mean ventricular volumes, maintain normal vascular volume, and maintain normal oxygen saturation. It is not true that merely forcing the heart into certain volumetric states at key times during the cardiac cycle is sufficient to achieve the desired equilibrium states of the cardiovascular system as a whole. Furthermore, the path to achieving these desired equilibrium states depends sensitively on the ventricular pressure-volume curve, which varies according to the Frank-Starling mechanism in response to changes in systemic parameters.

Therefore, it is desirable for an extra-cardiac assist device to supply energy to a heart in such a way that enables the heart-extra-cardiac actuation system to respond to varying cardiovascular parameters according to the Frank-Starling mechanism.

The present state of medical device technology makes it clinically inappropriate to instrument a heart sufficiently to verify that the long term ventricular pressure-volume variations follow the Frank-Starling curves. Even given this information, the corrective action depends on the time varying values for ventricular volume and pressure and their first and second time derivatives. Such an equation does not typically have an analytical solution, and given the closed loop nature of the cardiovascular system the response to a supplemental force is nonlinear and a solution is difficult to achieve iteratively. However, the present invention will disclose means for properly fixating a heart in an extra-cardiac assist device, means for supplying supplemental diastolic phase energy, and means for applying a spectrum of systolic energy forms intended to restore a normal Frank-Starling mechanism to a failing cardiovascular system sufficient to achieve physiologic hemodynamics, and in a manner that minimizes or eliminates trauma to the heart.

Cardiovascular catastrophe associated with acute failure of the heart, such as acute myocardial infarction, is particularly responsive to restoration of normal ventricular pressure-volume dynamics. In these patients restoration of a near-normal Frank-Starling mechanism provides the body with the means to achieve systemic equilibrium. It is therefore desirable to provide a means for rapid implantation of the physiological extra-cardiac assist device Cup. The Cup is optionally installed on the heart by applying vacuum to the interior apex of the Cup. This enables a non-traumatic and technically simple means of cardiac attachment of the Cup device in the patient and facilitates diastolic actuation.

The need for high-flow vacuum assisted attachment is minimized by the design of the physiological extra-cardiac assist device Cup, having a shell member that is somewhat oversize for the typical heart and using an inflatable annular ring that provides firm attachment at the A-V groove once the Cup is in place. Thus the system may be designed with a simpler vacuum source having a relatively low flow requirement.

To install the Cup, the heart is exposed by a chest incision. The Cup is positioned over the apex of the heart in a position such that the apex of the heart is partially inserted therein. A vacuum is applied to the interior apex of the Cup, thereby pulling the heart and the Cup together, such that the apices of the Cup and the heart, and the inner wall of the Cup and the epicardial surface of the heart are substantially in contact and ready for actuation. This procedure can be accomplished in minutes, and it is easy to teach to individuals with minimal surgical skill.

Optimal physiological extra-cardiac assist device performance requires that the Cup be sealed to the heart. It is desirable to provide a fitting mechanism proximal to the rim (base) of the cup so that measurement of the patient's heart dimensions is not required prior to implantation. Such a fitting mechanism should allow for a selection of cup size, if a variety of sizes are anticipated, based on easily determined clinical metrics such as patient surface area or weight.

In order to minimize myocardial damage and affect diastolic support the cup fit must enable the base of the cup to seal chronically in the region of the atrio-ventricular (AV) groove.

It is preferred that the liner move with the heart surface such that a point on the liner and an adjacent point on the epicardium stay adjacent throughout the cardiac cycle. There are several reasons for wanting to fix the heart in the device in this way. Firstly, heart motion relative to the liner is a source of epicardial abrasion.

Secondly, proteinaceous fluid, always present during implantation, is trapped in a thin layer between the heart and liner providing a lubricious interface. If the heart-device motion is minimal this protein will more quickly form soft adhesions between the heart and liner, thus reducing the level of vacuum required to retain the heart in the cup and eventually allowing for the vacuum to be removed entirely.

Thirdly, a vacuum applied to the heart will seat the apex of the heart at the interior bottom of the cup and also fix the sealing ring near the AV groove. Thus lateral pressure applied to the ventricles will not be translated to axial dilation of the heart. This is important because inside the heart, cords attach the aortic valve leaflets to apical locations within the left ventricle and axial dilation may strain the cords or damage the valve leaflets as well as potentially cause aortic valve insufficiency.

Fourthly, the prevention of heart-liner motion will prevent lateral motion of the heart. This occurs because the right ventricle blood volume is at a lower pressure than the left ventricular blood volume. Thus pressure applied to the left ventricle tends to collapse the right ventricle first, shifting the septum laterally. This is referred to as heart volley, and it is a typical feature of primary systolic extra-cardiac actuations but not of the physiological extra-cardiac assist device of the present invention.

As described briefly above, heart volley can adversely affect left-right flow balance. The alternative is a phenomenon known as over-driving the heart, where excess volume displacement occurs in the device with the intention of complete ejection of the blood volume in both ventricles. Over-driving is non-physiological, and the normal ejection fraction is around 50% of the total ventricular volume. In order to achieve this end, more time must be spent during systole than is natural, which tends to limit diastolic filling if the device is operated in synchrony with the native heart. Over-driving creates a new volumetric equilibrium point where the mean ventricular volume decreases. This effect not only reduces the effectiveness of primary systolic extra-cardiac actuations, but causes substantial intra-ventricular damage caused by the ventricular free wall rubbing against the heart septum.

Lastly, anterior and posterior fixation of the epicardium in lines corresponding to the location of the septum lessens septal bulging into the lower pressure right ventricle during early systole. This is another potential source of left-right flow imbalance that is not mitigated in prior art extra-cardiac assist devices.

Optimal physiological extra-cardiac assist device versatility and stability over a range of normal and pathological circulatory conditions requires energy be delivered to the heart in a variety of forms.

Proper fixation of the heart in the Cup, as described briefly above, allows for delivered forms of energy to retain their intended positional and temporal dependency and prevents supportive energy from being converted to damaging forms.

For example, epicardial motion of any kind relative to the liner generates heat. Heat is itself damaging, but more importantly is the byproduct of friction and stain. Fixation also presents the possibility of preferentially driving one ventricle more than the other. Although the right and left ventricles eject approximately the same volume, the left side stroke uses more energy because the change in pressure from end diastole to peak systole is greater. This is why the left free wall is many times thicker than the right free wall.

Therefore a heart-assist device is needed that does not cause damage to the heart as a result of its mechanical action on the heart (extra-cardiac assist devices) or implant requirements (blood contacting devices). There also exists a need for an active implantation means to ensure that such a device (1) is properly positioned and/or installed on the heart, (2) adequately seals against the heart, (3) achieves the desired systolic and diastolic action at installation and over the implanted life of such device, (4) supports the cardiovascular system in regaining the Frank-Starling mechanism needed to enable the patient to maximize the benefits of support, and (5) delivers a spectrum of energy forms critical in driving the cardiovascular system toward natural hemodynamic equilibrium, a condition necessary for successful weaning of the patient off the device.

There is also a need for a process to accomplish the above tasks very quickly, in order to avoid brain death and other organ damage. The inherent ability of the physiological extra-cardiac assist device Cup of the present invention to be installed in a very short period of time without surgical connection to the cardiovascular system of the patient enables the Cup of the present invention to save patients who require acute resuscitation. The active acquisition of the heart by the cup enables minimally invasive surgical implantation and reduces the need to visually expose the heart.

There is also a need for a device that does not contact the blood so that anticoagulation countermeasures are not needed, and so that the potential for infection within the blood is reduced.

It is therefore an object of this invention to provide a Physiological Extra-Cardiac Actuation Device that does not damage the heart as a result of its mechanical action on the heart or misappropriation of supportive energy.

It is a further object of this invention to provide a physiological extra-cardiac assist device that is technically straightforward to properly install and operate.

It is an additional object of this invention to provide a physiological extra-cardiac assist device that may be installed on the heart and rendered functional by a procedure that is accomplished in a few minutes.

It is another object of this invention to provide a direct physiological extra-cardiac assist device that adequately seals against the heart, thereby enabling intended operation of the device.

It is an additional object of this invention to provide a physiological extra-cardiac assist device that drives the systolic and diastolic action of the heart within precisely defined and controlled parameters via a spectrum of energy forms.

It is a further object of this invention to provide a physiological extra-cardiac assist device that supports the heart function in a state that is in normal equilibrium with the rest of the cardiovascular system in order to improve clinical outcome during reduction of support levels and eventual device removal.

It is another object of this invention to provide a physiological extra-cardiac assist device that provides appropriate feedback data necessary for the proper operation of the device and routine assessment of cardiovascular health while on support.

It is a further object of this invention to provide a physiological extra-cardiac assist device that provides functional data such as stroke volume, ejection fraction, actuation rate, % systole, and any parameter of clinical use relevant to maintaining support and course of therapy.

It is a further object of this invention to provide a physiological extra-cardiac assist device that does not require the implantation or placement of medical sensors that are not typically available; and, relies primarily on extra-corporal EKG for synchronization of the device to the native rhythm of the heart, in cases where such synchronization is warranted.

It is another object of this invention to provide a physiological extra-cardiac assist device that is compatible with devices intended to restore normal electrical function of the heart, including pacemakers and defibrillators and means for interfacing to such devices for the purpose of synchronization.

It is an object of this invention to provide a physiological extra-cardiac assist device that has no direct contact with circulating blood, nor requires surgical modification of the heart that results in exposure of blood thereby reducing the risk for thrombogenic and bleeding complications, thus decreasing the potential for infection of the blood, and eliminating the need for anticoagulation that has many serious complications, especially in patients with serious cardiovascular disease and recent surgery.

It is another object of this invention to provide passive means for assessing the phase synchrony between device and heart.

It is another object of the present invention to provide a physiological extra-cardiac assist device that can augment cardiac function without any surgical insult (direct or indirect) to the heart and/or great vessels.

It is another object of the present invention to provide a physiological extra-cardiac assist device that reduces the metabolic requirements of the heart while increasing blood flow to the vessels of the myocardium during positive diastolic actuation.

It is a further object of the present invention to provide a physiological extra-cardiac assist device having a detachable liner, which can thus facilitate passive fitting of the cup to the heart, stimulate the formation of soft bonds between the liner and the epicardium, increase the durability of the liner, and enable the physiological extra-cardiac assist device to be removed from the patient with no trauma to the heart of the patient.

It is a further object of the present invention to provide a physiological extra-cardiac assist device having a fluid-induced volumetrically expanding liner and/or seal, thereby improving fit, assisting in the dehydration of proteinaceous fluids, establishing an immediate tackiness, and providing a low durometer interface between heart and device.

It is a further object of the present invention to provide volumetrically expanding elements within the Cup that mitigate abrasion, occlusion of surface vessels, and damage to vascular grafts and friable areas of the myocardium while maintaining an intended fixation of the heart relative to the Cup.

It is a further object of the present invention to provide a physiological extra-cardiac assist device with Control System capable of controlling the time evolution of cup volume and pressure, the time derivative of cup volume and pressure, and the second time derivative of cup volume and pressure.

It is a further object of the present invention to provide a physiologic extra-cardiac assist device that is capable of both using as its primary drive control cup-pressure-derived data and alternatively using as its primary drive control cup-volume-derived data.

In accordance with the present invention, there is provided a process for assisting the function of a heart disposed within a body and comprising an outer wall, the process comprising the steps of measuring at least one parameter that is indicative of the function of the heart, applying a compressive force to a portion of the outer wall of the heart, and applying an expansive force to the portion of the outer wall of the heart.

In accordance with the present invention, there is further provided an apparatus for assisting the function of a heart disposed within a body and comprising an outer wall, the apparatus comprising a cup-shaped shell having an exterior wall, an interior wall, an apex, and an upper edge; a liner having an outer surface and an inner surface, an upper edge joined to the interior wall of the cup-shaped shell, and a lower edge joined of the interior wall of the cup-shaped shell, thereby forming a cavity between the outer surface thereof and the interior wall of the shell; and a drive fluid cyclically interposed within the cavity, the drive fluid applying a variety of forces on a portion of the outer wall of the heart.

In accordance with the present invention, there is further provided an apparatus for assisting the function of a heart disposed within a body, and comprising an outer wall, the apparatus comprising a cup-shaped shell having an exterior surface and an interior surface; a liner having an outer surface, an upper edge joined to the interior surface of the cup-shaped shell, and a lower edge joined of the interior surface of the cup-shaped shell, thereby forming a cavity between the outer surface thereof and the interior surface of the shell; a drive fluid cyclically interposed within the cavity; a volumetric expansive toroidally molded element position on the interior surface such that upon inflation the toroidally molded element fluidically seals the cup-shaped shell to the outer surface of the heart; the toroidally molded element coated with a fluidically expansive substance such that upon contact with fluids naturally present on the heart the fluidically expansive substance swells and induces the naturally present fluids to form a temporary bond between the toroidally molded element and the outer surface of the heart; the fluidically expansive substance also deposited on a location below the lower edge on the interior surface of the cup-shaped shell; and the combination of the fluidically expansive substances serve to fix the outer surface of the heart such that points on the liner adjacent to points on the outer surface of the heart remain at fixed distances during cyclical interposition of the drive fluid.

In accordance with the present invention, there is further provided an apparatus for assisting the function of a heart disposed within a body, and comprising an outer wall, the apparatus comprising a cup-shaped shell having an exterior surface and an interior surface; a liner having an outer surface, an upper edge joined to the interior surface of the cup-shaped shell, and a lower edge joined of the interior surface of the cup-shaped shell, thereby forming a cavity between the outer surface thereof and the interior surface of the shell; a drive fluid cyclically interposed within the cavity; the drive fluid possessing a compressive characteristic such that once the drive fluid is introduced into the cavity potential energy is applied to the outer surface of the heart; drive fluid conduit oriented relative to the exterior surface of the cup-shaped shell directing the drive fluid in a direction approximately perpendicular to the outer wall of the heart such that inertial energy is applied to the outer surface of the heart; the drive fluid possessing a volumetric characteristic such that once the drive fluid is introduced into the cavity baric energy is applied to the outer surface of the heart; and control means for repeatably delivering the three forms of energy in a prescribed ratio, and with prescribed relative timing.

The physiological extra-cardiac assist device of the present invention described above is advantageous because compared to other prior art devices, it utilizes inertia energy to precisely drive the mechanical actuation of the ventricular chambers of the heart without damaging the tissue thereof, or the circulating blood. Additionally the physiological extra-cardiac assist device uses at least one additional form of energy to ensure the physiological extra-cardiac assist device-heart system re-establishes the Frank-Starling mechanism so that the cardiovascular system of the patient can respond to system changes in volume and load; it may be installed by a simple procedure that can be quickly performed; it localizes the heart in the Cup so as to minimize heart volley and heart expulsion; it provides functional performance data of the heart; and it can provide electrophysiological synchronization and allow the heart to control actuation of the Cup.

As a result of the invention, a greater variety of patients with cardiac disease can be provided with critical life-supporting care, under a greater variety of circumstances, including but not limited to; enhanced capacity for resuscitation, bridging to other therapies, and extended or even permanent support because it utilizes a novel ratio of energy forms to support the heart and establish normal cardiovascular equilibrium. Finally the device can support the heart through a period of acute injury and allow healing, in some conditions, to full recovery of unsupported heart function, and places the recovering heart in a state uniquely suited for device explantation, this state is typically not achieved by other extra-cardiac assist device.

### BRIEF DESCRIPTION OF THE DRAWINGS

The invention will be described by reference to the following drawings, in which like numerals refer to like elements, and in which:

FIGS. 1-8 are graphical representations of time dependent pressure and volume relationships of blood displaced by the left and right ventricles of a healthy human heart, of an unhealthy human heart, and of a physiological extra-cardiac assist device assisted heart during systole and diastole;

FIGS. 9-10 are graphical representations of time dependent blood pressure within the left and right ventricles of a healthy human heart, and of a physiological extra-cardiac assist device assisted heart, respectively, during systole and diastole;

FIGS. 11-12 are graphical representations of time dependent blood flow rates ejected from the left and right ventricles of a healthy human heart, and of a physiological extra-cardiac assist device assisted heart during systole;

FIG. 13 is a graphical representation of time dependent blood flow rates into and out of the ventricles of the heart taken over a sequence of two physiological extra-cardiac assist device—assisted complete cardiac cycles;

FIGS. 14-18 are cross-sectional schematic views depicting a sequence of actions of physiological extra-cardiac assist device of the present invention a heart, which assist the systolic and diastolic functions thereof depicted graphically in FIGS. 1-13;

FIGS. 21-26 are cross-sectional schematic views depicting undesired operations and/or effects of a physiological extra-cardiac assist device, which is lacking the proper control and/or structural features provided in accordance with the present invention;

FIGS. 27-30 are cross-sectional schematic views depicting operations and/or effects of a physiological extra-cardiac assist device on a heart afflicted with pulmonary hypertension and right ventricular hypertrophy;

FIGS. 31-32 are cross-sectional schematic views depicting the action of a liner of a prior art extra-cardiac assist device upon the wall of the heart;

FIGS. 33-36 are cross-sectional schematic views depicting the action of the liner of one preferred physiological extra-cardiac assist device Cup of the present invention upon the wall of the heart;

FIG. 37 is a cross-sectional view depicting the action of the inflatable annular ring used to properly size the physiological extra-cardiac assist device Cup to a heart, and to retain the Cup in place;

FIG. 38 is a flow chart of a general method for using sensor data to guide physiological extra-cardiac assist device installation and assess cardiac performance under the influence of physiological extra-cardiac assist device;

FIGS. 39 and 40 are flowcharts of a more specific algorithm for automatically adjusting the function of an embodiment of the physiological extra-cardiac assist device Cup;

FIG. 41 is a schematic representation of working fluid pressure and/or flow rate sensors integrated into the Cup and Drive Assembly;

FIGS. 42 and 43 are two sectional views of the physiological extra-cardiac assist device Cup showing embodiments of a barrier membrane and a bio-adhesive attachment strip;

FIG. 44 is a schematic of an algorithm used to control flow balance in left and right ventricles;

FIG. 45 is a graph depicting the relationships between typical pressure and flow rates of drive fluid to the physiological extra-cardiac assist device Cup;

FIG. 46 is a graph depicting the relationship between inertial and total energy applied to the heart under a typical set of operational conditions;

FIG. 47 is a graph illustrating the effects of left-right balance on the Frank-Starling mechanism for a patient on physiological extra-cardiac assist device support;

FIG. 48 is a plot of the Frank-Starling mechanism under various operational conditions for the physiological extra-cardiac assist device Cup;

FIG. 49 is a graph of actual pressure-flow traces for a rabbit heart under various operational set points for the physiological extra-cardiac assist device Cup; and

FIG. 50 is another plot of actual rabbit pressure-flow data as operational changes from those in FIG. 49 to improve cardiac output.

### DETAILED DESCRIPTION

For a general understanding of the present invention, reference is made to the drawings. In the drawings, like reference numerals have been used throughout to designate identical elements.

In describing the present invention, a variety of terms are used in the description. Standard terminology is widely used in cardiac art. For example, one may refer to Bronzino, J. D., The Biomedical Engineering Handbook, Second Edition, Volume I, CRC Press, 2000, pp. 3-14 and 418-458; or Essential Cardiology, Clive Rosendorf M. D., ed., W.B. Saunders Co., 2001, pp. 23-699, the disclosures of which are incorporated herein by reference.

As used herein, the term Cup is meant to indicate the implanted portion of the physiologic extra-cardiac assist device of the present invention, such device comprising a cup-shaped outer shell. The term physiologic extra-cardiac assist device in this specification is intended to denote the overall physiological extra-cardiac assist device of the present invention in its various embodiments, unless specifically noted otherwise.

As used herein, the abbreviation LV is meant to denote the term “left ventricle,” or “left ventricular” and the term RV is meant to denote the term “right ventricle, or “right ventricular,” as appropriate for the particular context. “Right” and “left” as used with respect to the ventricles of the heart are taken with respect to the right and left of the patient's body, and according to standard medical practice, wherein the left ventricle discharges blood through the aortic valve into the aorta, and the right ventricle discharges blood through the pulmonic valve into the pulmonary artery

However, the Figures of the instant application, which depict the present invention and the heart contained therein, are taken as viewed facing the patient's body. Accordingly, in such Figures, the left ventricle depicted in any such Figure is to the right and vice-versa just as is done in convention when viewing radiographs and figures of related organs in the medical field. For the sake of clarity in such Figures, the left and right ventricles are labeled “LV” and “RV,” respectively.

As used herein, the terms “normal heart,” and “healthy heart” are used interchangeably, and are meant to depict a nominal, unafflicted human heart, not in need of DMVA assistance or other medical care.

As used herein, the term cardiac function is meant to indicate a function of the heart, such as the pumping of blood in systemic and pulmonary circulation; as well as other functions such as healing and regeneration of the heart following a traumatic event such as e.g., myocardial infarction. Parameters indicative of such functions are physical parameters, including but not limited to blood pressure, blood flow rate, blood volume, and the like; and chemical and biological parameters such as concentrations of oxygen, carbon dioxide, lactate, etc.

As used herein, the term cardiac state is meant to include parameters relating to the functioning of the heart, as well as any other parameters including but not limited to dimensions, shape, appearance, position, etc.

The physiologic extra-cardiac assist device of the present invention will now be described in detail. This description will begin with a description of the systolic and diastolic cycles of a healthy human heart, the systolic and diastolic cycles of an unhealthy human heart (of which there are many variants), and in general, how the physiologic extra-cardiac assist device of the present invention differs from prior art extra-cardiac assist devices.

In a subsequent description in this specification, the manner in which the physiologic extra-cardiac assist device of the present invention provides assistance to an unhealthy human heart on a long time scale according to various algorithms is provided. In some embodiments, such assistance entails heart health detection and a weaning strategy, such that the heart is assisted to an overall improved state and cardiovascular equilibrium is restored at which time physiologic extra-cardiac assist device support is gradually reduced and the device finally explanted.

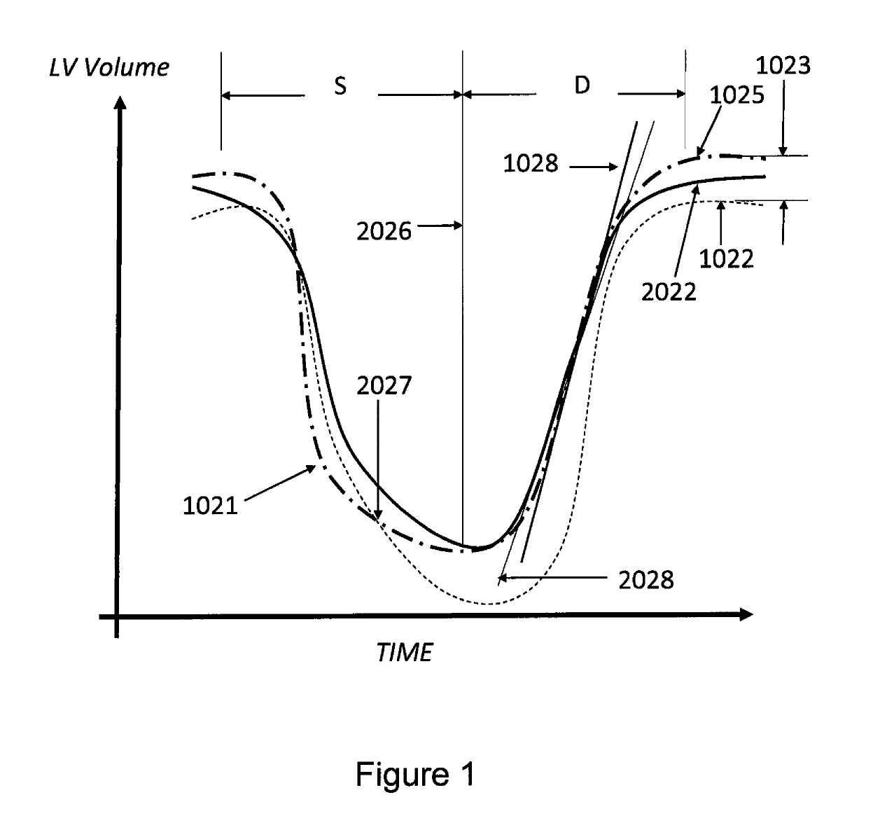

It is to be understood that the FIGS. 1-12, which depict time-dependent volumes, pressures, and flow rates of blood displaced by the ventricles of physiologic extra-cardiac assist device assisted hearts, prior art extra-cardiac assist assisted and non-assisted hearts are illustrative in nature, and are not meant to indicate precise quantitative values thereof, nor the sole beneficial functions thereof.

It is to be further understood that representations of such parameters with respect to an “unhealthy heart” are also illustrative in nature, and may vary widely, depending upon the particular cardiac disorder that is affecting such unhealthy heart, which can vary from incremental degrees of worsening dysfunction to cardiac standstill (“cardiac arrest”).

Accordingly, the particular representations of DMVA assistance to such exemplary unhealthy hearts are to be taken as one embodiment of assistance thereto, and that many other time dependent pressure, volume, and/or flow rate curves and resulting mechanical assistance can be provided by the DMVA device to such unhealthy or even non-beating hearts, which may be equally or more beneficial. A key attribute of the DMVA device of the present invention is the capability thereof to sense the performance of the heart and the performance of the device itself without the need for implanting sensors, and with embedded algorithms in the control system thereof, to select and execute a beneficial sequence of assistive actions to the heart to which it is fitted.

In the following description of FIGS. 1-12, references to ventricular volume are taken with respect to the blood volume contained within the ventricles, rather than blood volume displaced from the ventricles. Thus it will be apparent that blood volume in the ventricles is shown to decrease to a minimum at the completion of systole, and to increase to a maximum at the completion of diastole. Blood pressure is to be considered from a frame of reference within the ventricles unless noted otherwise. Also with regard to FIGS. 1-12 and in various subsequent Figures, the use of the upper case letter “S” is meant to indicate systole, and the use of the upper case “D” is meant to indicate diastole.

FIGS. 1-8 are graphical representations of time dependent pressure and volume relationships of blood displaced by the left and right ventricles of a healthy human heart, of an unhealthy human heart, and of a DMVA-assisted heart during systole and diastole.

FIG. 1 in particular is a representation of the time dependence of the volume of the left ventricle during one complete cardiac cycle including systole (S) and diastole (D), for a normal healthy heart, for a typical prior art extra-cardiac assist device assisted heart, and for one embodiment of a physiologic extra-cardiac assist device assisted heart.

Referring to FIG. 1, there is depicted the time dependent left ventricular volume curve 2020 (solid line) for a healthy heart, the time dependent left ventricular volume curve 1020 (dashed line) for an extra-cardiac assist device assisted heart, and the time dependent left ventricular volume curve 1021 (dot-dash-line) for a physiologic extra-cardiac assist device assisted heart.

Several preferred features of the physiologic extra-cardiac assist device and method of the present invention are illustrated in curve 1021 of FIG. 1.

In the preferred embodiment, the physiologic extra-cardiac assist device Cup is fitted to the heart such that the end diastolic volume 1025 of the physiologic extra-cardiac assist device assisted heart is slightly more (by volume difference 1023) than the end diastolic volume 2022 of a normal heart, and significantly more than the end diastolic volume 1022 of the extra-cardiac assist device assisted heart. In this manner, the end diastolic volume can be varied by control methodology (described later) from a super-normal state needed to reoxygenate ischemic tissue to a normal state as cardiovascular equilibrium is restored, and further if clinically useful to gradually train a dilated heart to a less dilated end ventricular volume.

No such freedom exists for the extra-cardiac assist device assisted heart since such devices typically do not contain a sealing ring and rely on end diastolic ventricular pressure to maintain a seal. Another beneficial feature is that the physiologic extra-cardiac assist device does not over drive the heart as does the extra-cardiac assist device 1022, 2022. Over driving the heart has been found in animal studies to result in endocardial abrasion.

There is also benefit to shifting earlier in time and extending the duration of end systole as achieved in 1021 (physiologic extra-cardiac assist device) as compared to shortening and shifting later in time as depicted in 1020 (extra-cardiac assist device). Shifting the actuation cycle earlier in time relative to the native contraction is the only way to access the restoration of native contractility and provide maximum contractile support. Coincident actuation is in practice difficult to achieve, impossible to monitor without implanting pressure or flow probes, and if slightly delayed fails to adequately rest the heart. In cases where active diastolic support is present, delayed systole loads the heart by resisting contraction, a condition that can rapidly increase the metabolic requirements of the heart and expand regions of recent myocardial ischemic damage.

A general feature of an extra-cardiac assist device apparatus and method is the propensity to compress the left ventricle to a lesser end systolic volume 1022 than the normal heart LV end-systolic volume 2022. This requires longer than normal filling time, but since end systole is shifted later in time there is both more volume to fill and less time to accomplish this end. The result typically is a gradually decreasing mean ventricular volume, which ultimately defeats the long term efficacy of the device. This condition also shifts blood volume into the distal vasculature placing the entire cardiovascular system out of equilibrium. Thus, although the cardiac cycle in extra-cardiac assist device assistance achieves a normal stroke volume the heart begins at a lower LV end diastolic volume 1022, it achieves a correspondingly lower LV end systolic volume, and thus does not represent normal physiologic heart function.

In the embodiment depicted in FIG. 1, the physiologic extra-cardiac assist device achieves end-systolic volume at a time 1027 of the actuating cycle slightly earlier than the time 2026 of a normal heart's cardiac cycle. Moreover, the physiologic extra-cardiac assist device ensures maximal support during LV compression by such relative earlier timing in this portion of the actuating cycle rather than increases in LV compression.

Thus, such a strategy does not interfere with adequate diastolic filling, and achieves a higher level of myocardial support during the systolic phase of the actuating cycle.

The physiologic extra-cardiac assist device is operated such that it also provides active assistance to the heart in diastole. Such active diastolic assistance provides for longer end systolic duration which has been shown in animal studies to improve myocardial circulation. Increased myocardial circulation beneficially affects myocardial healing. In some cases where the heart wall is abnormally thick it may be beneficial to increase the rate of diastolic filling above normal as is indicated by the steeper slope 1028 (change in volume/change in time or dV/dt) as compared to the normal filling rate 2028, which can be achieved with any extra-cardiac assist device possessing active diastolic support.

In addition, it can be seen that systolic emptying under physiologic extra-cardiac assist device support is improved over extra-cardiac assist device support as curve 1020 crosses curve 1021 at point 2027. Assistance is notably important to overcome pathological conditions that impair diastolic filling and constrain end-diastolic geometry. The sensing means and control algorithms and numerous structural features such as the Cup shell edge seal and apex restraint that are described subsequently in this specification enable this active diastolic assistance capability.

Such internal sensing means, algorithms, and features enable the physiologic extra-cardiac assist device and method to be adapted as required to provide assistance to an unhealthy heart in a manner that is optimal for the particular disorder afflicting such heart.

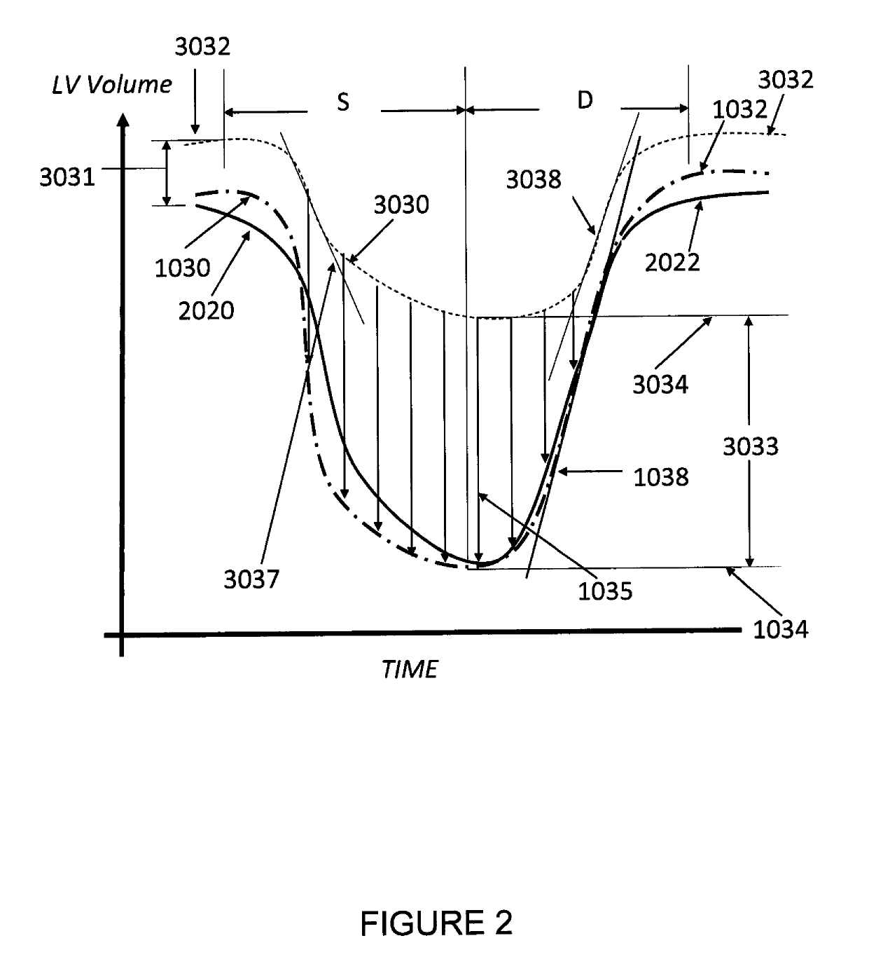

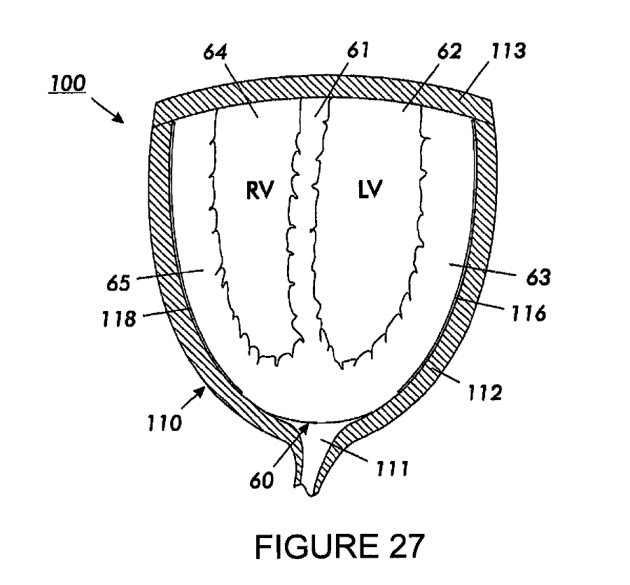

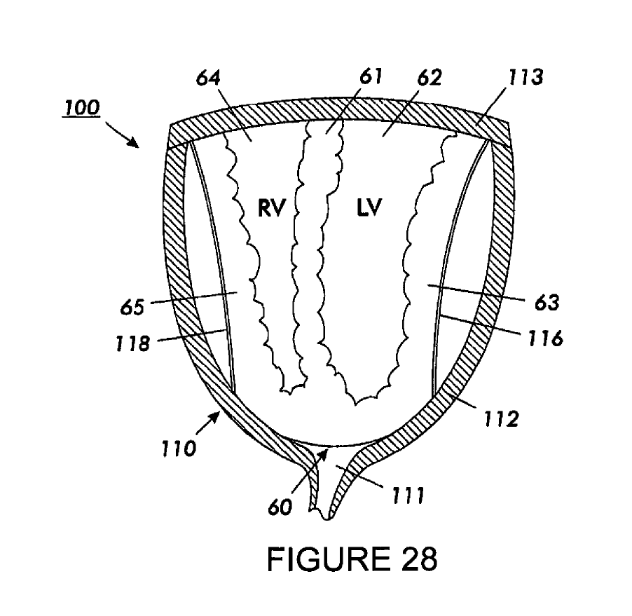

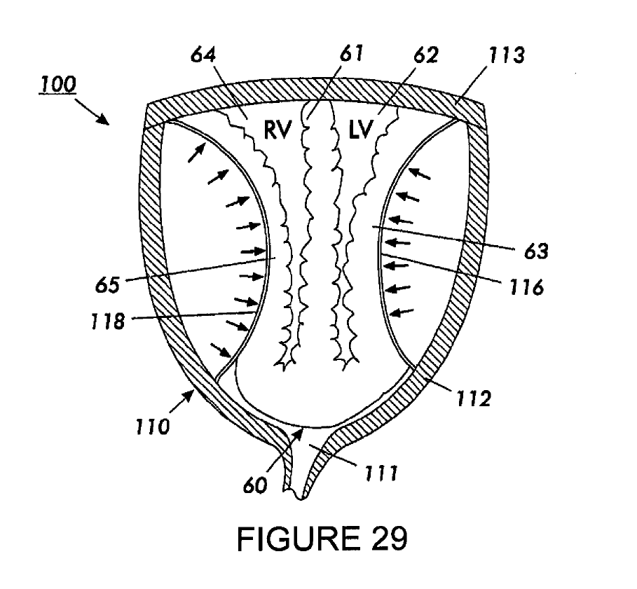

FIG. 2 is a representation of the time dependence of the volume of the left ventricle during one complete cardiac cycle including systole (S) and diastole (D), for a normal healthy heart, for a physiologic extra-cardiac assist device assisted heart immediately after implantation, wherein such heart is unhealthy and in a distended condition such as the heart depicted in FIGS. 27-29 and described subsequently in this specification, and for a physiologic extra-cardiac assist device assisted recovered heart prior to explantation, wherein such heart is less distended, the myocardium is rested, and native contraction has improved metabolic efficiency.

Referring to FIG. 2, curve 3030 (dashed line) represents the left ventricular volume of the unhealthy heart during a cardiac cycle, as compared to the LV curve 2020 (solid line) for a normal heart. It will be apparent that there is a substantial difference 3031 between the end diastolic volume 2022 of a healthy heart, and the end diastolic volume 3032 of the unhealthy, dilated heart in FIG. 2.

It will be further apparent that the volumetric output of such an unhealthy heart is much less than a normal heart, as indicated by the difference 3033 between the end systolic volumes thereof.

Curve 1030 (dot dashed line) depicts the LV volume of the assisted unhealthy heart immediately after implantation, which is provided assistance by the physiologic extra-cardiac assist device. Note the diastolic volume 3032 of the unhealthy heart is above the diastolic volume 2020 for a normal heart, which is compatible with the diastolic volume of the physiologic extra-cardiac assist device 1025 (FIG. 1) and not compatible with the diastolic volume of the extra-cardiac assist device 1020 (FIG. 1).

Referring back to FIG. 2, the physiologic extra-cardiac assist device is fitted and programmed to operate at a greater end diastolic volume 1032 than the normal heart 2022 and lesser than the end diastolic volume 3032 of the unhealthy heart, which benefits the unhealthy heart by reducing myocardial stretch and/or wall tension.

The embodiment depicted in FIG. 2, illustrates that physiologic extra-cardiac assist device support of the unhealthy, dilated heart operates at a higher end diastolic volume than the end diastolic volume 2022 of an otherwise normal beating heart. Ventricular remodeling during assistance may allow the physiologic extra-cardiac assist device assisted heart to achieve lower end-diastolic volumes that may benefit the heart by improving its chance for recovery. The physiologic extra-cardiac assist device assisted heart immediately achieves end systolic volume(s) 1034 that are significantly less than the end systolic volume 3034 of the unhealthy unassisted heart in order to effectively improve stroke volume and improve total cardiac output.

Thus a substantial difference in output between the unhealthy heart and the assisted heart is achieved, as indicated by the relative area 1035 between curves 1030 and 3030. It will be apparent that the net stroke volume output of the assisted heart is approximately the same as that of a healthy heart albeit at an elevated mean LV volume. Adjustments in support level can be varied by adjustments in drive dynamics as deemed appropriate to both minimize myocardial stress and achieve optimal ventricular dynamics. At a later time, and just before explant, the physiologic extra-cardiac assist device assisted heart 1030 approximates the normal heart function in terms of stroke volume, end diastolic volume and end systolic volume.

These functional characteristics are achieved while “tailoring” the fit and operation of the physiologic extra-cardiac assist device to the particular unhealthy heart in a manner that does not damage such heart while providing assistance thereto.

In the embodiment depicted in FIG. 2, the unhealthy heart is provided with active assistance during systole and diastole, as indicated by the relatively steep slopes 1037 and 1038, respectively, of curve 1030 as compared to the relatively gradual slopes 3037 and 3038, respectively of curve 3030 for the unassisted unhealthy heart.

One method of assessing myocardial health is to gradually decrease the Cup pressure equivalent of slope 1037, the inflation pressure rate, until there appears a change in slope, indicating where the assist-driven slope is overcome by the healthier contractility of the native heart. In the extreme case, the Cup pressure will actually drop as the heart attempts to pull away from the Cup liner.

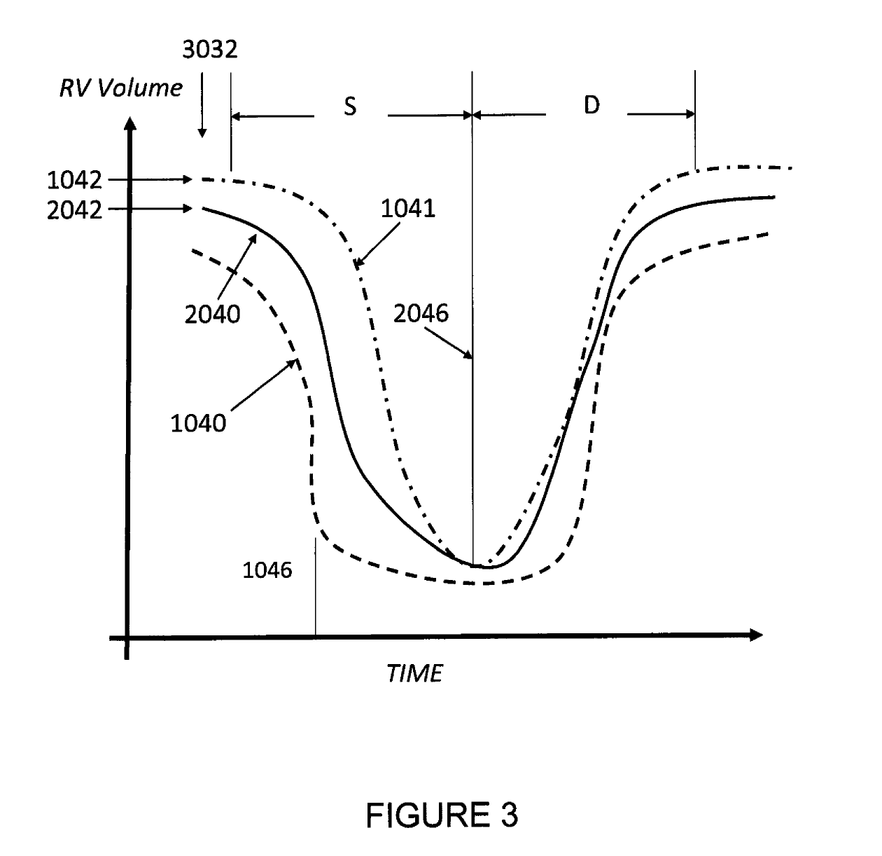

FIG. 3 is a representation of the time dependence of the volumetric changes of the right ventricle during one complete cardiac cycle for a normal healthy heart, an extra-cardiac assist device assisted heart, and for a physiologic extra-cardiac assist device assisted heart.

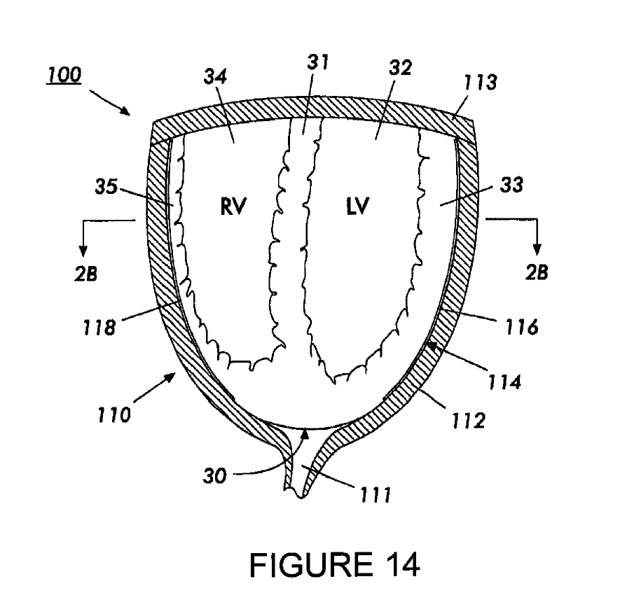

Referring to FIG. 3, there is depicted the time dependent right ventricular volume curve 2040 (solid line) for a healthy heart, the time dependent right ventricular volume curve 1040 (dashed line) for an extra-cardiac assist device assisted heart, and a time dependent right ventricular volume curve 1041 (dot-dashed line) illustrated in general in FIGS. 14-30 and subsequently described in this specification.

In the DMVA embodiment depicted in FIG. 3, some similar preferred features are illustrated in curve 1041, as were depicted in curve 1021 of FIG. 1. In the preferred embodiment, the volume of the physiologic extra-cardiac assist device Cup and the displacement of the liner therein are fixed to the heart such that the heart does not move relative to the liner surface. This condition does not exist in extra-cardiac assist devices, even those with vacuum attachment. Vacuum resists predominately axial motion of the heart relative to the liner; it does not prevent the heart from sliding side to side.

The RV pressure is significantly less than the LV pressure, therefore any uniform circumferential force will cause the heart to displace in the cup in such a way that the LV will be displaced away from the expanding liner and the RV will be pressed into the expanding liner, thus the RV will collapse before the LV and before 1040 its normal time course 2040 as depicted in FIG. 3.

The details of this mechanism will be described subsequently. While the physiologic extra-cardiac assist device of the present invention employs heart localization to achieve a better balance between right and left ventricular compression, in a preferred embodiment the actuating fluid is delivered on the left side of the Cup cavity and delayed by structural members in its propagation to the right side of the Cup cavity.

A left-right adjustment means is further provided that involves adjusting the ratio of the magnitude of supportive energy forms, such that a higher ratio of inertial to baric energy favors LV compression, and a lower ratio of inertial to baric energy favors RV compression. There is a variety of techniques for assessing flow imbalance clinically that do not require implantation of additional sensors, although in emergent situations the preferred actuation delays the start of compression 1041 relative to the normal time dependent volume curve 2040. It may also be desirable to shorten the interval during which the RV is at minimum volume, in order to reduce elevated mean right atrial pressure, which elevates mean venous pressure, and ultimately forces the cardiovascular system out of normal equilibrium.

In contrast, the RV end diastolic volume 1042 of the extra-cardiac assist device assisted heart is less than the RV end diastolic volume 2042 of a normal heart, which contributes to an early RV actuation relative to the normal. Additionally, the extra-cardiac assist apparatus will compress the right ventricle to a lesser end systolic volume than the normal heart RV end systolic volume, in many cases to a volume which allows the endocardial surface of the right free wall to abrade against the septum.

In similar fashion, the RV is delayed in filling since the higher filling pressure of the LV will cause the LV to fill the decreasing volume of the interior of the Cup at a pressure higher that needed to start RV filling. This effect is defeated in the physiologic extra-cardiac assist device design by a variety of mechanisms, the most important mechanism being the removal of the inertial energy component during Cup evacuation (diastole).

Thus, as indicated by the sequence of FIGS. 14-20 depicting an extra-cardiac assist device assisted heart, the systolic actuation and corresponding displacement of blood from the right ventricle begins substantially in advance of and is completed before the corresponding displacement of blood from the left ventricle.

In the embodiment depicted in FIGS. 3 and 1, systolic actuation of the right ventricle is relatively complete at a time 1046 that is substantially earlier and/or more rapid than the normal RV ejection time that comparatively ends at time 2046. The overall time for RV ejection is thereby relatively abbreviated while the duration of the systolic interval is relatively increased. During the time interval 1049 required to complete DMVA assisted systole for the left ventricle and to begin diastole, the right ventricular free wall is squeezed, fixed and maintained in a position contacting the septum at a relatively constant end systolic volume 1044.

Subsequently, active diastolic assistance is provided to the right ventricle, as for the left ventricle assistance described and shown in FIG. 1. It will be apparent that the slope 1048 (change in volume/change in time or dV/dt) of curve 1040 for the extra-cardiac assist and physiologic extra-cardiac assist device assisted heart is generally steeper than the corresponding slope 2048 of curve 2040 for the normal heart for right ventricular diastolic actuation, as was previously noted for the left ventricular diastolic actuation.

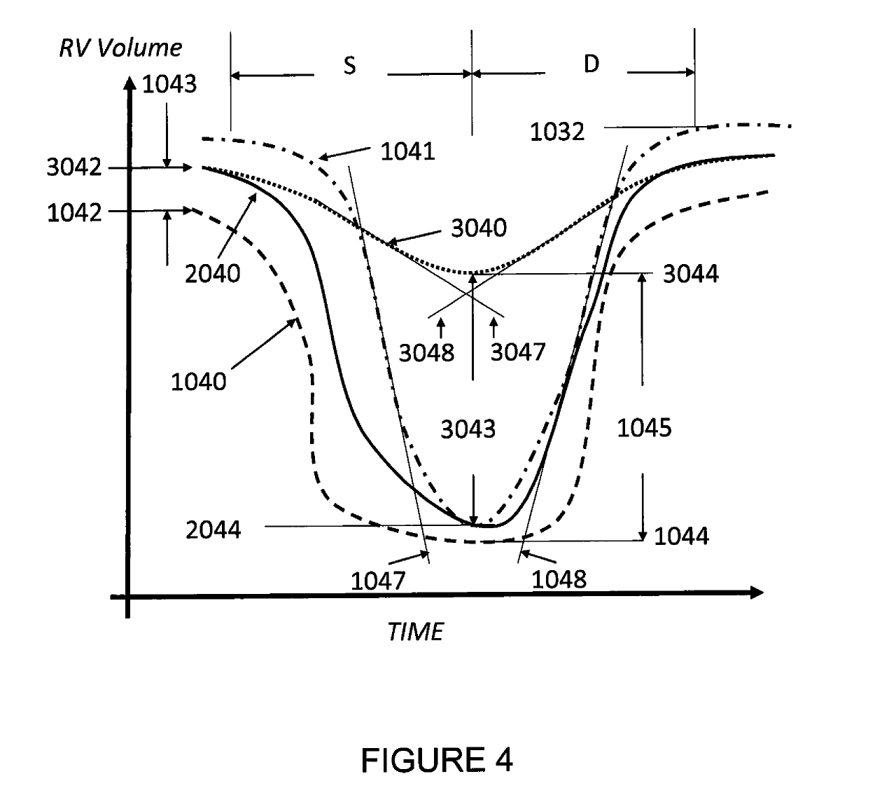

FIG. 4 is a representation of the time dependence of the volume of the right ventricle during one complete cardiac cycle for a normal healthy heart (curve 2040, solid line), for an embodiment of an extra-cardiac assist device assisted heart (curve 1040, dashed line), and for a physiologic extra-cardiac assist device assisted heart (dot-dashed line 1041, dashed line), wherein such heart is unhealthy.

Referring to FIG. 4, curve 3040 (dotted line) represents the right ventricular volume of the unhealthy heart during a cardiac cycle, as compared to the RV curve 2040 for a normal heart. It will be apparent that the volumetric output of such an unhealthy heart is much less than a normal heart, as indicated by the difference 3043 between the end systolic volumes thereof. Curve 1040 depicts the condition of RV over driving; the curve depicts the RV volume of the assisted unhealthy heart, which is provided assistance by an extra-cardiac assist device.

In the embodiment depicted in FIG. 4, the extra-cardiac assist device is fitted and programmed to operate at a slightly lesser end diastolic volume 1042 and the physiologic extra-cardiac assist device is fitted and programmed to operate at a slightly greater end diastolic volume 1032 than the end diastolic volume 3042 of the unhealthy heart, as shown by volume 1043. Both the extra-cardiac assist and physiologic extra-cardiac assist device assisted heart can achieve end systolic volumes 1044 and 2044, respectively, that are significantly less than the end systolic volume 3044 of the unhealthy unassisted heart in order to obtain an adequate stroke volume.

The difference being that the physiologic extra-cardiac assist device can achieve the same stroke volume at a greater end systolic volume, thus providing greater margin to protect against RV free wall contact with the septum. In both cases a substantial difference in output between the unhealthy heart and the assisted heart is achieved, as indicated by the difference 1045 between end systolic volumes 1044 and 3044.

In the embodiment depicted in FIG. 4, the end systolic volume 1044 of the extra-cardiac assist device assisted heart is less than the end systolic volume 2044 of a normal heart; however the physiologic extra-cardiac assist device substantially matches the end systolic volume 2044 of a healthy heart, such that the net RV blood volume output of the assisted heart is approximately the same as that of a healthy heart. This balanced output is achieved while “tailoring” the ratio of energy forms the physiologic extra-cardiac assist device delivers to the particular unhealthy heart in a manner that does not damage such heart while providing assistance thereto. In the embodiment depicted in FIG. 4, the unhealthy heart is provided with active assistance during systole and diastole, as indicated by the relatively steep slopes 1047 and 1048 as compared to the relatively weak slopes 3047 and 3048 of curve 3040 for the unassisted unhealthy heart.

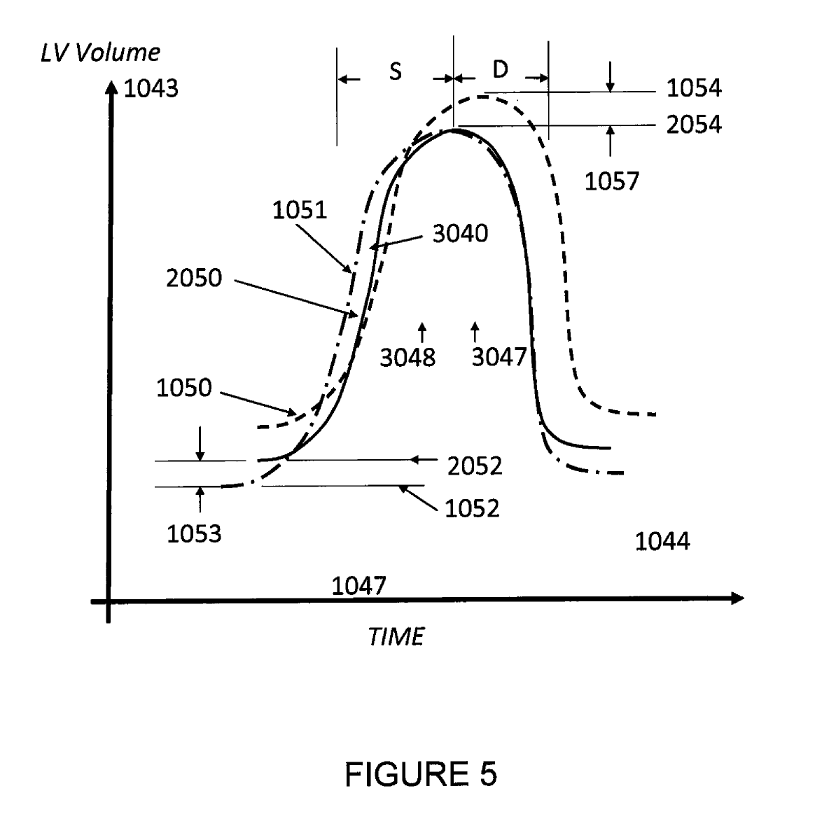

FIG. 5 is a representation of the time dependence of the blood pressure within the left ventricle during one complete cardiac cycle for a normal healthy heart (solid line), for an extra-cardiac assist device assisted heart (dashed line) and for a physiologic extra-cardiac assist device assisted heart (dot-dash line). The curve representing the extra-cardiac assist device assisted heart is “shifted” slightly to the right relative to the normal heart. Physiologic extra-cardiac assist device assistance of the heart begins before natural contraction of the heart to reduce the work of the heart.

With this understanding, FIG. 5 depicts the time dependent left ventricular pressure curve 2050 for a healthy heart (solid line), the time dependent left ventricular pressure curve 1050 (dashed line) for a extra-cardiac assist device assisted heart, and the time dependent left ventricular pressure curve 1051 (dot-dash line) for a physiologic extra-cardiac assist device assisted heart, illustrated in general in FIGS. 14-30 and subsequently described in this specification.

In the preferred embodiment, the physiologic extra-cardiac assist device Cup is slightly larger than the heart, with the displacement of the liner therein such that the very early diastolic pressure 1052 of the physiologic extra-cardiac assist device assisted heart is less than the very early diastolic pressure 2052 of a normal heart. This is not shown in pressure difference 1053. For the extra-cardiac assist device assisted heart, the end-diastolic pressures are increased, as illustrated in pressure difference 1053, which reflects the fit (Cup smaller than heart) of the extra-cardiac assist device and its physical effect on ventricular pressures in the normal heart.

Another preferred feature of the physiologic extra-cardiac assist device apparatus and method is the ability thereof to increase stroke volume without elevating the left ventricle to a greater peak systolic pressure 1054 than the normal heart LV maximum systolic pressure 2054. This is beneficial from the point of view of damage to the heart ventricles as well as left/right flow balance. Yet another preferred feature is the ability to attain greater relative increases and decreases in the rate of pressure change (dP/dt) as indicated by slope 1056 without increasing peak systolic pressure, when compared to a healthy heart. The larger size of the physiologic extra-cardiac assist device enables it to be more effectively matched to the requirements of the particular unhealthy heart needing assistance and also does not make the heart dependent upon the device, which is the likely result if the heart volume were to be reduced from its pre-implant volume. The physiologic extra-cardiac assist device apparatus of the present invention is thus atraumatic with respect to the heart.

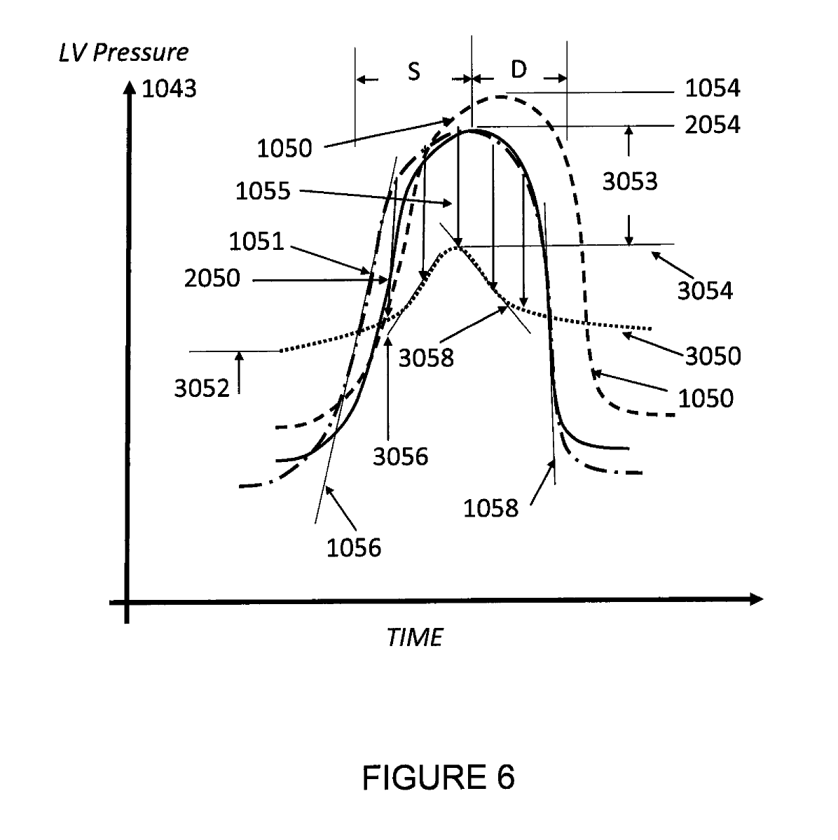

FIG. 6 is a representation of the time dependence of the pressure of the left ventricle during one complete cardiac cycle for a normal healthy heart, for an extra-cardiac assist device assisted heart, and for a physiologic extra-cardiac assist device assisted heart, wherein such heart is unhealthy. Referring to FIG. 6, curve 3050 (dotted line) represents the left ventricular pressure of the unhealthy heart during a cardiac cycle, as compared to the LV curve 2050 (solid line) for a normal heart. It will be apparent that the LV pressure of such an unhealthy heart is much less than a normal heart, as indicated by the difference 3053 between the peak systolic pressures thereof, 2054 and 3054. Curve 1050 (dashed line) depicts the LV pressure of the assisted unhealthy heart, which is provided assistance by an extra-cardiac assist device and curve 1051 (dot-dash line), which is provided assistance by a physiologic extra-cardiac assist device.

In the embodiment depicted in FIG. 6, the physiologic extra-cardiac assist device is fitted and programmed to operate at a lower diastolic pressures 1052 than the diastolic pressure 3052 of the unhealthy heart. The amount of work performed by the extra-cardiac assist device (vertical hash) and the physiologic extra-cardiac assist device (horizontal hash) are approximately equivalent, as indicated by the region 1055 between pressure curves 1050 and 3050 and between 1051 and 3050. This is followed by a more rapid decrease in pressure (dP/dt) as indicated by slope 1058 of curve 1051.

Thus, in the embodiment depicted in FIG. 6, the unhealthy heart is provided with active assistance during systole and diastole, as indicated by the relatively steep slopes 1056 and 1058 as compared to the relatively weak slopes 3056 and 3058 of curve 3050 for the unassisted unhealthy heart. As indicated previously, such values of dP/dt for the physiologic extra-cardiac assist device assisted heart, while significantly greater (i.e. steeper in slope) than those of an unassisted unhealthy heart they are adjusted to be somewhat more approximate to the overall characteristics of those for a healthy heart.

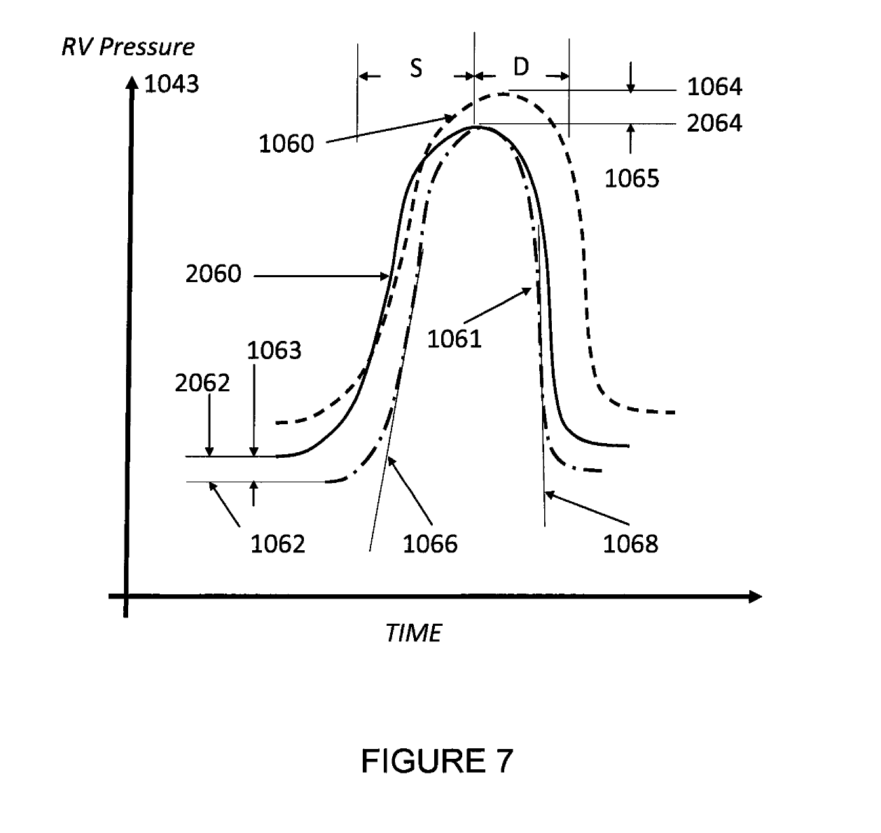

FIG. 7 is a representation of the time dependence of the blood pressure within the right ventricle during one complete cardiac cycle for a normal healthy heart, for an extra-cardiac assist device assisted heart, and for a physiologic extra-cardiac assist device assisted heart.

Referring to FIG. 7, there is depicted the time dependent right ventricular pressure curve 2060 (solid line) for a healthy heart, the time dependent right ventricular pressure curve 1060 (dashed line) for a extra-cardiac assist device assisted heart, and the time dependent right ventricular pressure curve 1061 (dot-dash line) for a physiologic extra-cardiac assist device assisted heart, illustrated in general in FIGS. 14-30 and subsequently described in this specification.

In the preferred embodiment, the physiologic extra-cardiac assist device Cup is fitted to the heart, and the displacement of the liner therein is controlled such that the RV diastolic pressure 1062 of the physiologic extra-cardiac assist device assisted heart is slightly less (by pressure difference 1063) than the RV diastolic pressure 2062 of a normal heart.

An extra-cardiac assist apparatus and its method of use are dependent on a higher peak systolic pressure 1064 than the normal heart RV maximum systolic pressure 2064. It can be seen that the pressure difference 1065 between these peak systolic pressures is even greater than the corresponding difference 1057 between the peak systolic pressure 1054 of the assisted heart and the peak systolic pressure 2054 of the normal heart (see FIG. 5).

This greater difference is due to the additional pressure needed to displace blood from the left ventricle. Such an increased pressure, which is provided by the extra-cardiac assist fluid drive system, occurs during the time that the RV free wall is in contact with the septum, thus maximizing the potential for damage. The higher peak systolic pressures 1064 of the extra-cardiac assist device assisted heart are reflected into the pulmonary circulation as well as increased RV stroke volume relative to LV stroke volume and both tend to produce an increase in pulmonary blood pressure within the patient. This increased pulmonary blood pressure can lead to edema.

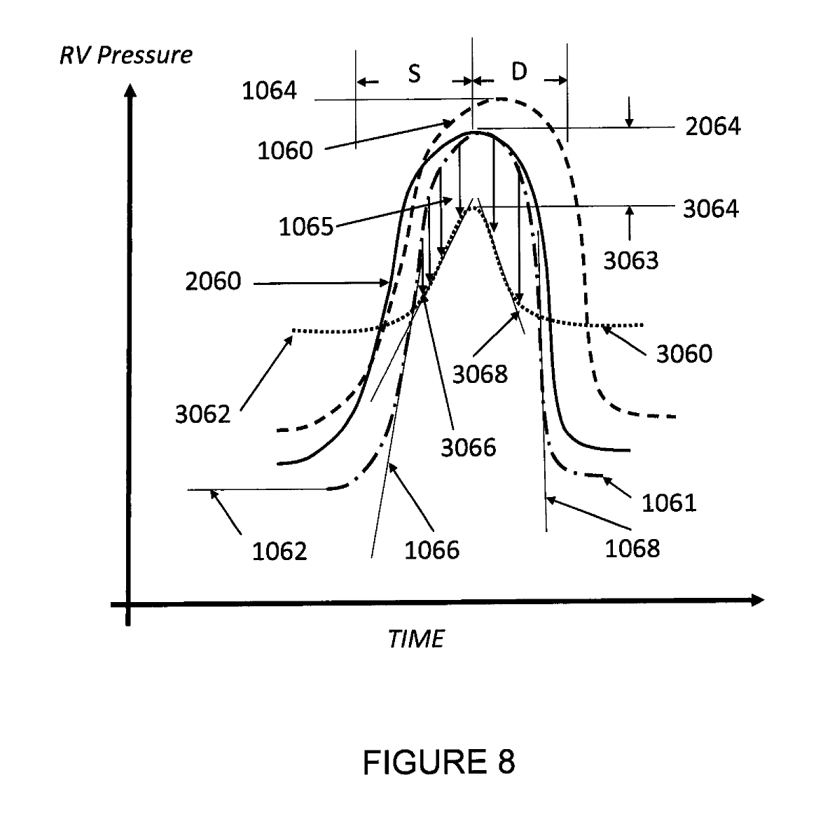

FIG. 8 is a representation of the time dependence of the pressure of the right ventricle during one complete cardiac cycle for a normal healthy heart, for an extra-cardiac assist device assisted heart, and for a physiologic extra-cardiac assist device assisted heart, wherein such heart is unhealthy.

Referring to FIG. 8, curve 3060 (dotted line) represents the right ventricular pressure of the unhealthy heart during a cardiac cycle, as compared to the RV curve 2060 (solid line) for a normal heart. It will be apparent that the RV systolic pressure of such an unhealthy heart is much less than a normal heart, as indicated by the difference 3063 between the peak systolic pressures thereof. Curve 1060 (dashed line) depicts the RV pressure of the extra-cardiac assist-assisted unhealthy heart, and curve 1061 (dot-dash line) depicts the RV pressure of the physiologic extra-cardiac assist device assisted unhealthy heart, which is provided assistance by these devices.

In the embodiment depicted in FIG. 8, the physiologic extra-cardiac assist device is fitted and programmed to operate at a lower early diastolic pressure 1062 than the early diastolic pressure 3062 of the unhealthy heart. However, the physiologic extra-cardiac assist device assisted heart achieves a peak RV systolic pressure 2064 that is significantly greater by 3063 than the peak RV systolic pressure 3064 of the unhealthy unassisted heart and less than the extra-cardiac assist-assisted peak RV systolic pressure 1064.

Additionally, a substantial difference in pressure between the unhealthy heart and the assisted heart is maintained for a lesser portion of the cardiac cycle, as compared to the extra-cardiac assist device assisted heart indicated by the region 1065 between systolic pressure curves 1060 and 3060. This feature, in particular, is important in maintaining left/right flow balance. The peak systolic pressure is followed by a more rapid decrease in pressure (dP/dt) as indicated by slope 1068 of curve 1061.

Thus, in the embodiment depicted in FIG. 8, the unhealthy heart is provided with active assistance during systole and diastole, as indicated by the relatively steep slopes 1066 and 1068 as compared to the relatively weak slopes 3066 and 3068 of curve 3060 for the unassisted unhealthy heart. As indicated previously, such values of dP/dt for the physiologic extra-cardiac assist device assisted heart, while significantly greater (i.e. steeper in slope) than those of an unassisted unhealthy heart, are more closely representative of those for a healthy heart.

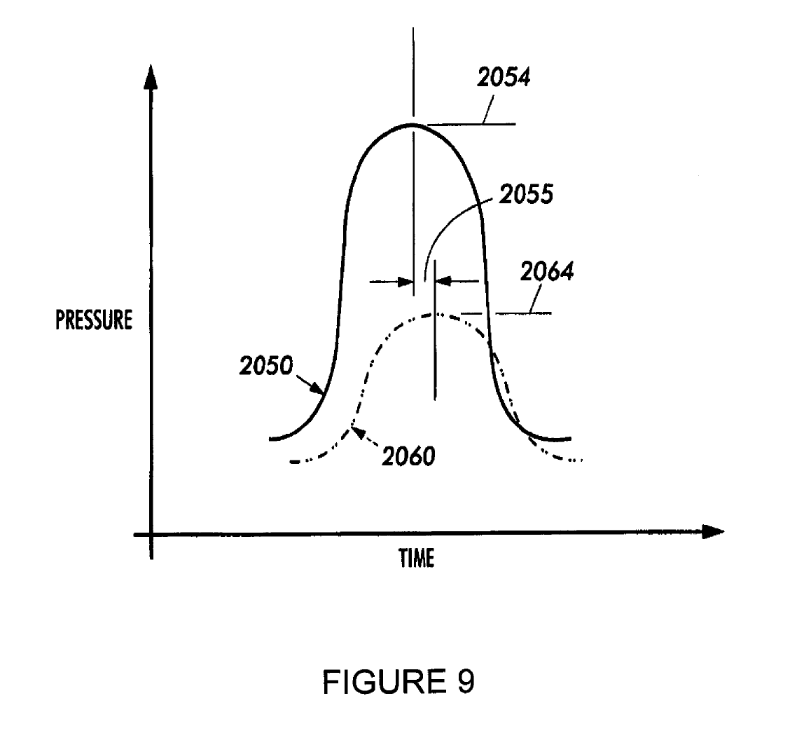

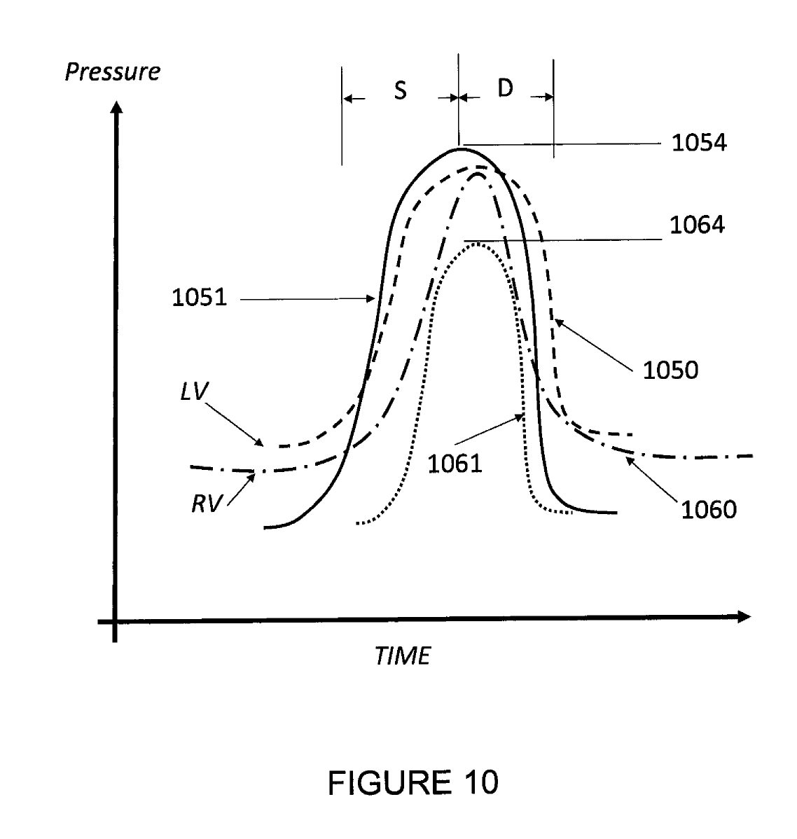

FIGS. 9 and 10 are graphical representations of time dependent blood pressure within the left and right ventricles of a healthy human heart, and of a DMVA-assisted heart during systolic and diastolic actuation.

Referring to FIG. 9I, which depicts the left ventricle pressure 2050 (solid line) and the right ventricle pressure 2060 (dash/double-dot line) for a healthy heart on the same time axis, it can be seen that the peak systolic pressure 2054 of the left ventricle is considerably higher than the peak systolic pressure 2064 of the right ventricle. It can also be seen that there is typically a small time difference 2055 between the occurrence of the peak systolic pressure 2054 of the left ventricle and the peak systolic pressure 2064 of the right ventricle.

FIG. 10 depicts the left ventricle pressure 1050 (dashed line) and the right ventricle pressure 1060 (dash/dot line) of a heart assisted by an extra-cardiac assist apparatus and the left ventricular pressure 1051 (solid line) and the right ventricular pressure 1061 (dot line) of a heart assisted by a physiologic extra-cardiac assist device apparatus.

Referring to FIG. 10, it can be seen that pressure increases occur approximately simultaneously, since the physiologic extra-cardiac assist device drive fluid is applying the same uniform pressure through the action of the liner therein to both ventricles. However, the early systolic slope of 1051 is considerable earlier than the slope of 1061, this is due to the application of inertial energy to the LV and the absence of this inertial energy applied to the RV. Nevertheless, the baric energy (pressure) drives the peak systolic pressures 1054 of the left ventricle and 1064 of the right ventricle so that the peaks occur at approximately the same time.

The partition between inertial and baric energy forms is such that the overall pressure rise of the RV is shifted to later times (right) compared to the normal beating heart. It will also be apparent the peak systolic pressure 1054 of the left ventricle is considerably higher than the peak systolic pressure 1064 of the right ventricle. This difference in pressures can only be achieved by applying different forms of energy to the left and right ventricles.

Otherwise, as depicted in curves 1050 and 1060, the peak LV and RV pressures are the same, which is characteristic of pure baric driven support. The difference in peak LV systolic pressure 1050 and RV systolic pressure 1064 is critical for left/right flow balance since higher pressure is needed for systemic circulation as compared to pulmonary circulation.

It can also be seen that the minimum right ventricle diastolic pressure is substantially lower than the corresponding minimum left ventricle diastolic pressure for the extra-cardiac assist device assisted heart. This is characteristic of a device constrained heart. For the physiologic extra-cardiac assist device assisted heart, the minimum end diastolic pressures for right and left sides are the same. The two pressures must be the same if cardiovascular equilibrium is to be achieved.

With regard to FIGS. 9 and 10, it is to be understood that there is no intent that such Figures are depicted on the same time scale, and that the cardiac cycle of an assisted heart occurs on approximately the same time scale as for the cardiac cycle of a normal heart.

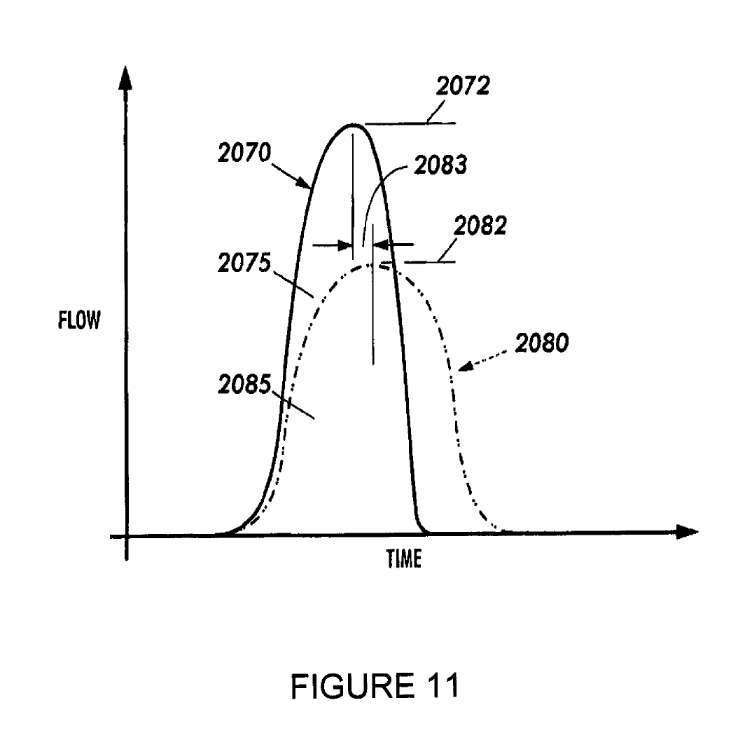

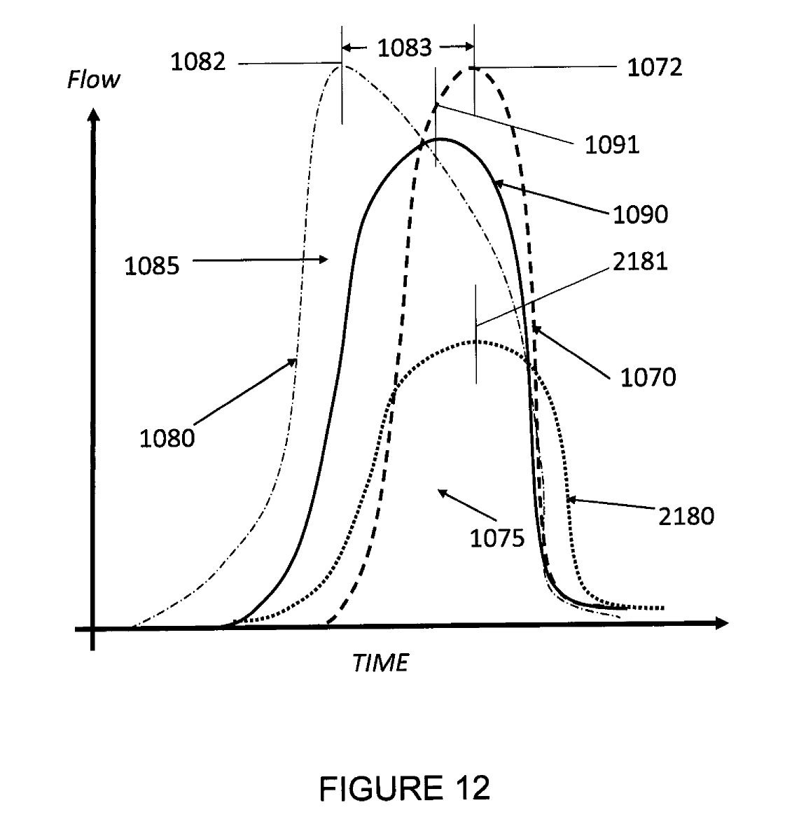

FIGS. 11 and 12 are graphical representations of time dependent rates of blood flow exiting from the left and right ventricles of a healthy human heart, of an extra-cardiac assist device assisted heart during systole and of a physiologic extra-cardiac assist device assisted heart during systole.

Referring to FIG. 11, which depicts the blood flow rate 2070 (solid line) from the left ventricle and the blood flow rate 2080 (dash/double-dot line) from the right ventricle for a healthy heart on the same time axis, it can be seen that the ejections are nearly concurrent, with the peak flow 2072 from the left ventricle preceding the peak flow 2082 from the right ventricle by a small interval 2083.

It can also be seen that the flow for the right ventricle occurs over a somewhat longer time interval, and that the area 2075 representing the total volume displaced from the left ventricle is approximately equal to the area 2085 representing the total volume displaced from the right ventricle, since the volume of systemic circulation is approximately equal to the volume of pulmonary circulation, with some variation due to the physiologic shunting of blood. It is also noted that these relationships will vary in accordance with different cardiovascular disease states.

FIG. 12 depicts the blood flow rate 1070 exiting from the left ventricle and the blood flow rate 1080 exiting from the right ventricle of a heart assisted by an extra-cardiac assist apparatus and the blood flow rate 1090 exiting from the left ventricle and the blood flow rate 2180 exiting from the right ventricle of a heart assisted by a physiologic extra-cardiac assist device apparatus.

Referring to FIG. 12 and the extra-cardiac assist device assisted heart, it can be seen that the ejections are not concurrent, but that the ejections overlap to some degree. The peak flow 1082 from the right ventricle precedes the peak flow 1072 from the left ventricle by interval 1083.

It can also be seen that, unlike the function of a normal heart, the majority of flow from the left ventricle occurs over a somewhat shorter time interval, and the area 1085 representing the total volume displaced from the right ventricle is considerably greater than the area 1075 representing the total volume displaced from the left ventricle.

Thus the volume of systemic circulation is far from equal to the volume of pulmonary circulation in an extra-cardiac assist—assisted heart. Referring to FIG. 12 and the physiologic extra-cardiac assist device assisted heart, it can be seen that the left (solid line) and right (dot line) ejections are roughly concurrent, with the left ventricular ejection slightly preceding the right ventricular ejection as it does in the normal heart.

The left ventricular flow peak 1091 slightly precedes the right ventricular flow peak 2181, as occurs in the normal heart. Most importantly, the area (stroke volume) of the left ventricular flow curve 1090 is substantially equal to the area of the right ventricular flow curve 2180. It should also be understood that these relationships will vary in accordance with different cardiovascular disease states. Accordingly, the ratio of inertial to baric energy forms can be adjusted to ensure these normal heart relationships are maintained.

With regard to the timing of blood flows of the extra-cardiac assist-assisted heart, it can be seen by reference to FIGS. 14-30 (to be subsequently explained in detail in this specification) that the extra-cardiac assist apparatus compresses and empties the right ventricle prior to the time at which such apparatus compresses and empties the left ventricle and in a relatively abbreviated time span within any given comparative cycle rate when contrasted to the normal beating heart. As previously explained, the precedence of the right ventricle is due to the timing of the pulmonary and aortic valve openings, and because the nominal pulmonary blood pressure is lower than the nominal aortic blood pressure and also due to the generally less resistant, thin RV wall when compared to the thicker LV free wall and septum.

With regard to the timing of blood flows of the physiologic extra-cardiac assist device assisted heart, it can be seen by reference to FIGS. 14-30 (to be subsequently explained in detail in this specification) that the physiologic extra-cardiac assist device apparatus compresses and empties the left ventricle prior to the time at which such apparatus compresses and empties the right ventricle. As previously explained, the precedence of the left ventricle is achieved by applying inertial energy to the left ventricle before substantial pressure rise in the liner.

This inertial energy is converted to baric energy before impacting the right side of the device. In the early phase, there is only inertial energy applied to the heart, since the below ambient condition of the interior of the cup equilibrates to ambient with the first interval of flow into the cup. The interior pressure in the cup must rise above ambient (or chest pressure if the chest is closed) for the heart to be supported. This situation is considerably enhanced for the physiologic extra-cardiac assist device where the cup is slightly larger than the heart and considerably blocked for the extra-cardiac assist device where the cup is smaller than the heart.

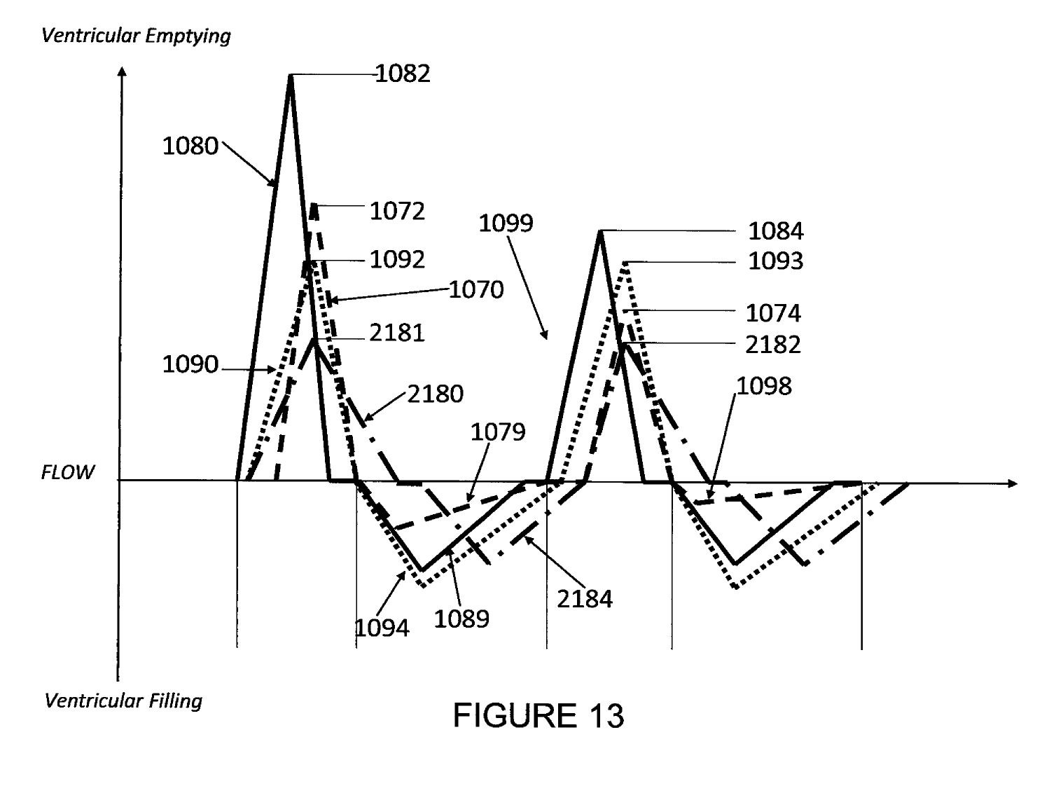

FIG. 13 is a graphical representation of time dependent blood flow rates into and out of the ventricles of the heart assisted by an extra-cardiac assist device and a physiologic extra-cardiac assist device taken over a sequence of two complete cardiac cycles.

Referring to FIG. 13, there is depicted for an extra-cardiac assist device assisted heart an overall left ventricle flow plot 1098 (dashed line) and an overall right ventricle flow plot 1099 (solid line) for two cycles. Left ventricle flow plot 1098 comprises curves 1070 (dashed line, one per cycle) during systole, and right ventricle flow plot 1099 comprises curves 1080 (solid line, one per cycle) during systole, each with flow out of the ventricle being taken as a positive value. Left ventricle flow plot 1098 further comprises curves 1079 (dashed line, one per cycle) during diastole, and right ventricle flow plot 1099 comprises curves 1089 (solid line, one per cycle) during diastole, each with flow out of the ventricle being taken as a negative value.

There is also depicted for a physiologic extra-cardiac assist device assisted heart an overall left ventricular flow plot 1090 (dot line) and an overall right ventricular flow plot (dot-dash line) 2180 for two cycles. Left ventricle flow plot 1090 further comprises curves 1094 (dot line, one per cycle) during diastole, and right ventricle flow plot 2180 comprises curves 2184 (dot-dash line, one per cycle) during diastole, each with flow out of the ventricle being taken as a negative value.

The physiologic extra-cardiac assist device curves demonstrate a cardiovascular system in equilibrium, and the extra-cardiac assist curves demonstrate a transitory cardiovascular state. For example, for the extra-cardiac assist device assisted heart peak RV systolic flow in cycle 1 (1082) is greater than peak systolic flow in cycle 2 (1084). Similarly, for extra-cardiac assist device assisted heart peak LV systolic flow in cycle 1 (1072) is greater than peak systolic flow in cycle 2 (1074).

The long term results are a progressively shrinking heart, which has an asymptote far from the normal heart volume. Referring to the physiologic extra-cardiac assist device assisted heart peak RV systolic flow in cycle 1 (2181) is approximately equal to the peak systolic flow in cycle 2 (2182). Similarly, for physiologic extra-cardiac assist device assisted heart peak LV systolic flow in cycle 1 (1092) is approximately equal to the peak systolic flow in cycle 2 (1093).

It is to be understood that the plots of FIG. 13 are for general illustrative purposes only, and that interpretation of details thereof are not intended to be taken as limiting.

For example, the sharp reversals of flow depicted at the apices of curves 1070, 1080, 1079, and 1089 occur in practice as smooth, curved transitions when the time line is expanded or the recordings are made with a greater speed. In addition, there may be a pause of relatively greater duration than indicated between the completion of ventricular filling, and the next cycle of ventricular emptying which are dictated by adjustments in the drive dynamics used to operate the devices. In general, the time scale of an assisted cardiac cycle is between about 0.5 and 1.0 seconds (120-60 beats per minute). Moreover, such variations in cycle rates will result in relative changes in the pressure and flow characteristics. However, it is to be understood that all of these variables, as well as many others are fully controllable in accordance with the present invention.

Referring again to FIG. 13, it can be seen that the ejections of blood from the right and left ventricles for the extra-cardiac assist device are not concurrent, but that such ejections do overlap to some degree, as depicted in FIG. 12.

The ejections of blood from the right and left ventricles for the physiologic extra-cardiac assist device are approximately concurrent. Although, during the extra-cardiac assist-assistance depicted in FIG. 13, the filling of the left and right ventricles are substantially concurrent.

The physiologic extra-cardiac assist device assistance depicted in FIG. 13 the filling of the left and right ventricles are not concurrent, the right ventricle filling later. This is primarily due to the higher filling pressure of the left atrium. The physiologic extra-cardiac assist device can be adjusted to create more rapid filling in the early part of diastolic actuation such that the filling of the right and left ventricles would be even more facilitated in the early part of diastolic actuation by applying successively more negative pressure until the desired filling and balance between left and right ventricles is achieved.

In certain circumstances, such as a rapidly beating heart, this may be advantageous, as it enables the controller to utilize more time in systolic compression if these were for example required to more appropriately compress the ventricles in the latter half of the cycle. The converse is also true: that is the controller could effectively empty the ventricles more rapidly, and based on the evaluation of the pressure and flow curves, thereby dedicate more time to diastolic actuation to ensure adequate filling. All of these adjustments require the evaluation of the resultant RV and LV volumes to ensure appropriate filling and emptying of the ventricles in each half of the cycle.

As can be seen from the relative timing and amplitudes of the curves depicted in FIG. 13, the physiologic extra-cardiac assist device is biased toward less filling and less stroke volume of the RV relative to filling and ejection of the LV. Conversely, the extra-cardiac assist device is strongly biased toward less filling and less stroke volume of the LV relative to filling and ejection of the LV. The present invention can be adjusted in degrees in a direction towards favoring the right ventricle by reducing the ratio of inertial energy to baric energy. This follows from the observation that the extra-cardiac assist device is a pure baric energy driven device.

In one embodiment to be described subsequently in this specification, the ventricular emptying and ventricular filling blood flows are inferred from volumetric changes of the cavity comprised of the Cup liner and shell by measuring the pressure and flow of drive fluid delivered to and from such device. In another embodiment, specific left and right blood flows are obtained by sensors in the pulmonary artery (RV) and descending aorta (LV). (In the latter case, correction factors must be applied to account for blood flow out of the brachiocephalic, left common carotid, and left subclavian arteries.)

FIGS. 14-30 are cross-sectional schematic views depicting a sequence of actions of a physiologic extra-cardiac assist device of the present invention on a heart, which assists the systolic and diastolic functions thereof depicted graphically in FIGS. 1-13.

For the sake of simplicity of illustration, only the ventricular portion of the heart that is contained in the physiologic extra-cardiac assist device Cup is shown in FIGS. 14-30; the atria and valves are not shown, with it being understood that such portions of the heart remain functional as commonly understood. Also for the sake of simplicity of illustration, the liner of the physiologic extra-cardiac assist device Cup, which displaces the ventricles to perform systolic and diastolic actuation, is shown as a simple membrane joined to the Cup shell wall. It is to be understood that numerous other liner embodiments of the present invention, as described and shown in this specification, are to be considered within the scope of the description of FIGS. 14-30.

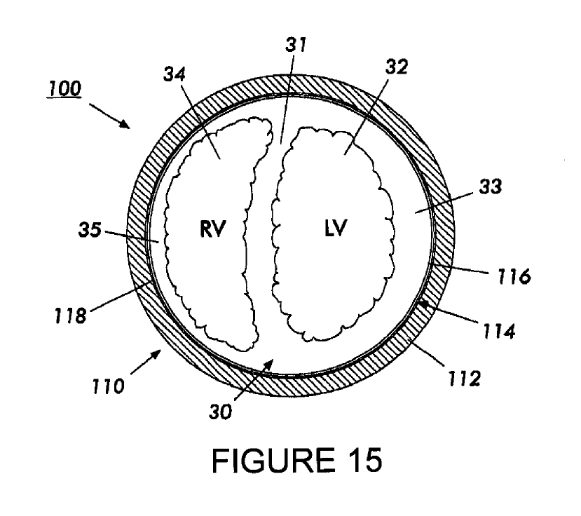

FIG. 14 is a cross-sectional elevation view of a heart in an uncompressed state contained within the physiologic extra-cardiac assist device Cup prior to the beginning of systolic compression, and FIG. 15 is a top cross sectional view taken along line 2B-2B of FIG. 14.

The relative timing of the situation of FIG. 14 in the cardiac cycle is shown by arrow 2A of FIG. 12. Referring to FIGS. 14 and 15, heart 30 comprising left ventricle 32 and right ventricle 34 is contained and secured within DMVA cup 100 by the action of vacuum drawn from tube 111 and by seal 113. The actual interface between tube 111 and seal 113 is not shown.

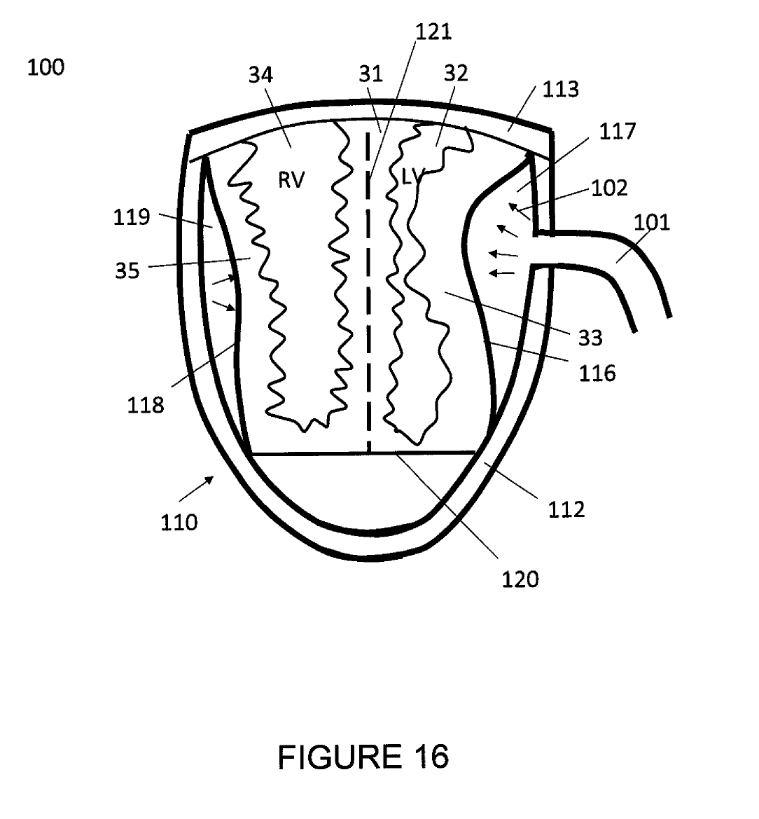

Physiologic extra-cardiac assist device Cup 100 further comprises a housing 110 with dynamic properties formed by wall 112, and elastic liner 114 attached to wall 112. In operation, a drive fluid is used to displace liner 114, with liner 114 preferably being of unitary construction, comprising a left portion 116 and a right portion 118. Such drive fluid displaces a continuous annular cavity between liner 114 and the inner surface of shell wall 112. Such annular cavity comprises a left cavity portion 117 (see FIG. 16) and a right cavity portion 119 (see FIG. 16). Thus the ventricular chambers of the heart are circumferentially compressed with the left ventricular free wall 33 of heart 30 being displaced by the left liner portion 116, and the right ventricular free wall 33 of heart 30 being displaced by right liner portion 118.

FIG. 16 is a cross-sectional elevation view of a heart contained within the physiologic extra-cardiac assist device Cup early in the process of systolic compression, approximately at the time indicated by arrow 2C of FIG. 12. Referring to FIG. 16, physiologic extra-cardiac assist device drive fluid is delivered into a supply port 101 in shell wall 112 and displaces liner 114, accumulating in cavity portion 119. The early displacement of liner 114 predominantly compresses left ventricular wall 33 of the heart 30, causing blood to flow from left ventricle 32 as indicated in FIG. 12 and described previously.

It this time, the pressure in left cavity portion 117 and right cavity portion 119 is approximately less than or equal to ambient and the motion of liner 114 is substantially due to the inertial energy of inflow jet 102. Substantially all of the inertial energy of jet 102 is transferred to left cavity portion 117 and LV 32. It is further true that the pressure in left cavity portion 117 and right cavity portion 119 tend toward equality during the evolution of systole, yet at pressures below a threshold, inertial energy exceeds baric energy, even during pressurization of right cavity portion 119 and compression of RV 34. Furthermore, change in left ventricular volume exceeds change in right ventricular volume as long as ((ΔLVV*LVP)−(ΔRVV*RVP))<inertial energy, where ΔLVV is the instantaneous change in LV volume, LVP is the left ventricular pressure, ΔRVV is the instantaneous change in RV volume, RVP is the right ventricular pressure, and inertial energy is the inertial energy applied to the left ventricle 32.

Furthermore, motion of the heart relative to the Cup is constrained at lip 113 and seal 120, and more specifically septum 31 is substantially restrained from translation by these restraints, and by the vacuum between the liner and the heart wall. The liner is anchored to the shell at location 121 anteriorly and posteriorly, such that the liner does not lift off the interior of the shell during inflation.

Anchor 121 is perforated by channels 122 such that drive fluid can pass from left cavity portion 117 to right cavity portion 119. The dimensions of channels 122 can be tailored to cup size or other clinical parameters to further favor actuation of the LV over the RV by introducing a delay in the transmission of baric energy from left cavity portion 117 to right cavity portion 119.

Typically, left cavity portion 117 will be at a higher pressure transitionally relative to right cavity portion 119 due to a pressure drop across anchor 121. In all these respects, the cross section length of septum 31 is unchanging due to the intervening vacuum and the fixity of the liner along the line 121

This is yet another distinguishing feature over extra-cardiac assist devices where when right ventricle wall 33 has been initially displaced by liner portion 116, intraventricular septum 31 has also been displaced toward right ventricle 34 both in lateral translation and bulging of septum 31 into RV 34. Accordingly, in the instance of extra-cardiac assist the left ventricle 32 has not exhibited any volume reduction by extra-cardiac assist drive fluid, and blood flow from left ventricle 32 has therefore not begun, also indicated at time 2C of FIG. 12.

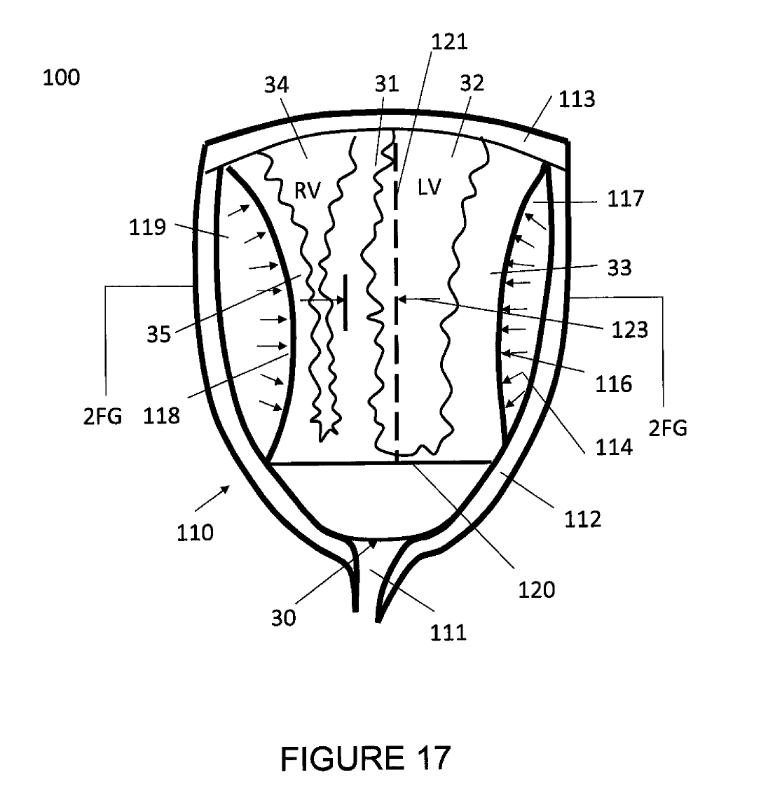

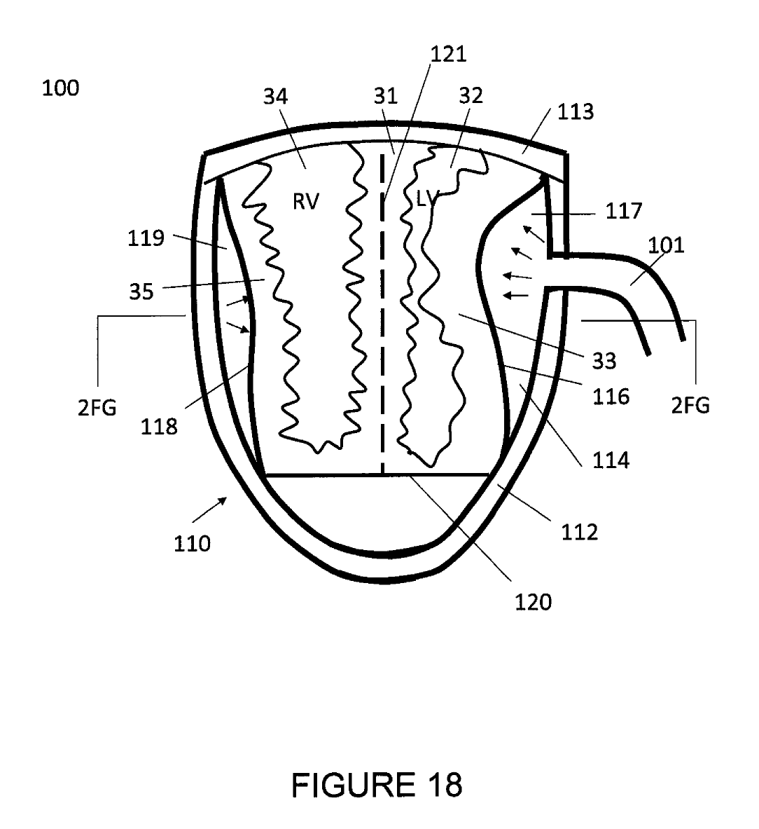

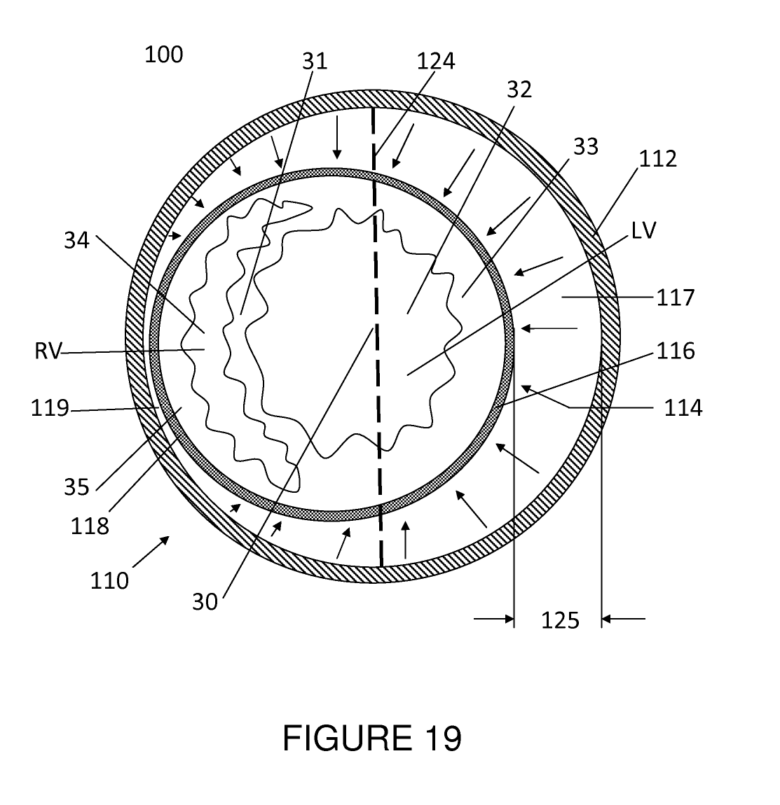

FIG. 17 is a cross-sectional elevation view of a heart contained within a extra-cardiac assist Cup at roughly the mid-point of systolic compression, and FIG. 18 is the same view within a physiologic extra-cardiac assist device Cup; and FIG. 19 is a top cross sectional view taken along line 2FG-2FG of FIG. 17, and FIG. 20 is a top cross sectional view taken along line 2FG-2FG of FIG. 17, approximately at the time indicated by arrow 2D of FIG. 12.

Referring to FIGS. 18 and 19, extra-cardiac assist drive fluid continues to flow into a supply port (not shown) in shell wall 112 into cavity portions 117 and 119, further displacing right ventricular wall 35 and left ventricular wall 33 of heart 30. The right ventricular volume is substantially smaller than the left ventricular volume, partially resulting from septum 31 bulging into the lower pressure RV as depicted by 123. It can be seen that left liner portion 116 provides compression forces on the left ventricular wall 33 of heart that lead to a greater reduction in volume of right ventricle 34. Accordingly, blood flows concurrently from right ventricle 34 and left ventricle 32 as indicated in FIG. 12 and described previously.

It can also be seen that in the extra-cardiac assist embodiment, the extra-cardiac assist apparatus applies a force uniformly to the heart around the circumference thereof, causing the preferential collapse of the right ventricle such that the heart is compressed in a manner that renders the heart with a substantial departure from a circular cross section and with a minimum diameter at the plane defined by line 2FG-2FG of FIG. 17. As used in this specification, the term cardiac core diameter is meant to indicate this diametrical minimum of the heart that occurs during extra-cardiac assist assistance.

Referring to FIGS. 18 and 20, physiologic extra-cardiac assist device drive fluid flows into a supply port 101 above line 2FG-2FG and near the greatest cross section of the ventricular volumes. The supply port 101 delivers fluid flow in shell wall 112 into cavity portions 117 and 119, displacing left ventricular wall 33 with inertial and elevated baric energy and right ventricular wall 35 with decreased baric energy. The left ventricular volume is slightly smaller than the right ventricular volume, partially resulting from fixation of septum 31 along line 121 preventing bulging into the lower pressure RV as depicted in FIG. 18.

It can be seen that left liner portion 116 provides inertial and baric forces on the left ventricular wall 33 of heart that lead to a greater reduction in volume of left ventricle 32. Accordingly, blood flows concurrently from right ventricle 34 and left ventricle 32 as indicated in FIG. 12 and described previously.

It can also be seen that in the physiologic extra-cardiac assist device embodiment, the physiologic extra-cardiac assist device apparatus applies a differential force that caused uniform compression to the heart around the circumference thereof, causing in the limit equal collapse of the right ventricle and left ventricle such that the heart is compressed in a manner that renders the heart with a substantially circular cross section and with a core diameter at the plane defined by line 2FG-2FG of FIG. 17 substantially normal, and aligned with the central plane 124 of the physiologic extra-cardiac assist device Cup.

In the extra-cardiac assist case depicted in FIGS. 18 and 19, there is both septal bulging 123 and lateral translation of the whole heart 125, such that the right ventricular free wall is in contact with the septum before substantial reduction in LV volume.

As depicted in FIGS. 18 and 20, there is neither septal bulging nor lateral translation of the heart which enables the inertial plus baric forces applied to the left ventricle to generate a reduction in LV volume approximately equal to the reduction in RV volume achieved with lower baric force.

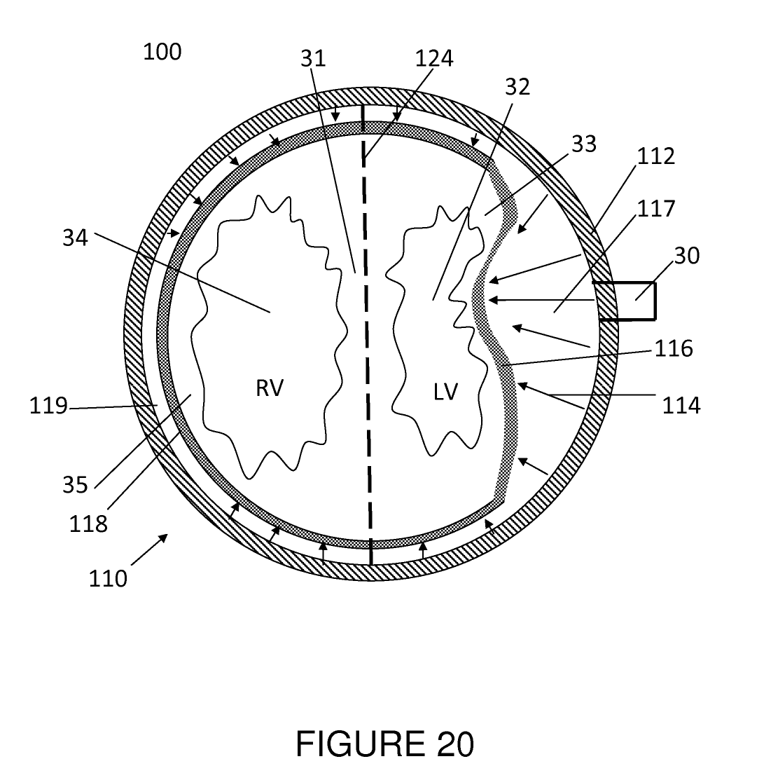

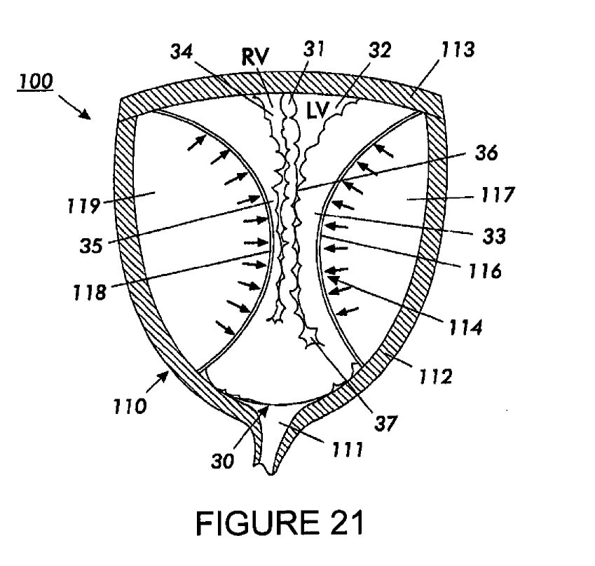

FIG. 21 is a cross-sectional elevation view of a heart contained within an extra-cardiac assist Cup at the completion of systolic compression. Referring to FIG. 21, right ventricle wall 35 has remained squeezed against intraventricular septum 31, left ventricle wall 33 has been nearly displaced to a point of contact with intraventricular septum 31, and blood flow from left ventricle 32 has ceased (see FIG. 12).

FIG. 21 is illustrative of a single mode constraint existing in all extra-cardiac assist devices. In order to achieve close to a flow balanced condition both right and left ventricles must be substantially completely evacuated. This condition is far from the normal in which 50% ejection fraction for normal hearts is typical, and far less for enlarged hearts. The ejection of this much blood volume causes the systemic circulation to experience cyclic hyper and hypobaric conditions, the fluctuations are typically outside the ability of the cardiovascular system to accommodate.

Frequently the response is a progressive reduction in total blood volume, which results in a lower mean arterial pressure. In all physiologic responses, the final state is far from a normal sustainable state without support, thus making the failing heart dependent upon the device.

In contrast, FIG. 17 is typical of an end systolic state, wherein a variety of ejection fractions can be achieved by changing the systolic duration, or the ratio of inertial to baric energy, or a variety of other strategies. Flow balance does not depend on total evacuation of the heart, and flow balance is maintained approximately from early systole to end systole, whereas flow balance in the extra-cardiac assist device is only achieved at higher than normal pressure and briefly at end systole.

FIGS. 21-26 are cross-sectional schematic views depicting undesired operations and/or effects of an extra-cardiac assist device, which typically cannot be avoided by control features alone and in accordance with the present invention require flow and structural features for normal heart support. Such conditions are additionally avoided by use of the internal sensing, controls, and algorithms of the present invention.

Referring to FIG. 21, there is depicted a heart 30 in a state of excessive compression by an extra-cardiac assist device 100. It can be seen that excessive forces are placed on the entire ventricular mass with the left ventricle 32 excessively compressed to a point where there is a large region 36 of contact between left ventricle wall 33 and intra-ventricular septum 31. In some instances, entrapment of blood may occur in a pocket 37 formed at the base of left ventricle 32.

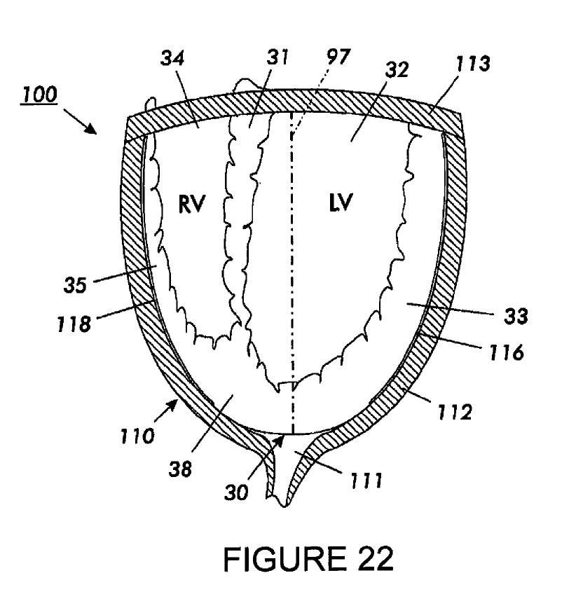

In all cases, and particularly in instances where the extra-cardiac assist Cup 100 is undersized for the particular heart 30, misalignment of the heart within the Cup occurs as depicted in FIG. 22, wherein the heart is shown at the conclusion of diastolic actuation.

Referring to FIG. 22, it can be seen that the right ventricle 34 has been substantially displaced from alignment with the central plane 97 of Cup 100, and that apex 38 of heart 30 has been displaced upwardly away from vacuum tube 111. Such a misalignment distorts predominantly the right ventricle 34, and prevents proper operation of the extra-cardiac assist Cup 100. RV filling in particular is compromised.

Such a circumstance is prevented by use of sufficient vacuum applied at vacuum port 111, but without a positive sealing ring at lip 113 the Cup pressure rapidly equilibrates with chest pressure by leakage thus the retaining force is lost.

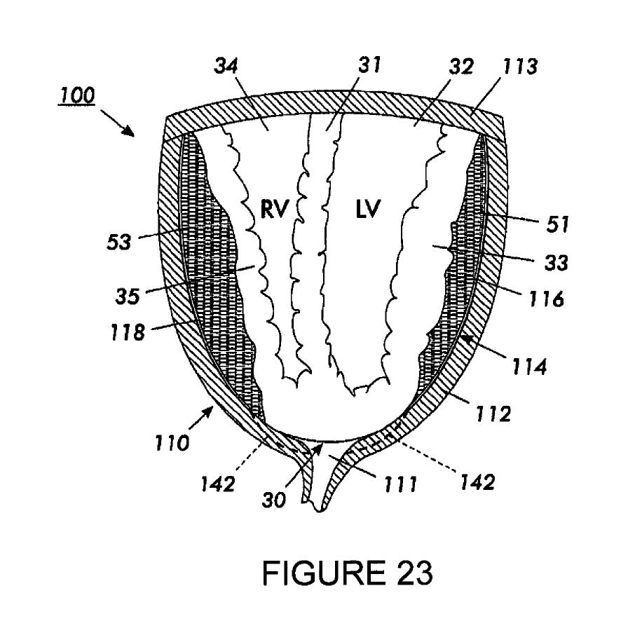

The result of leakage is depicted in FIG. 23, which depicts a situation wherein a type of “cavitation” has occurred during diastolic actuation, such that the left ventricle wall 33 and right ventricle wall 35 have become detached and are no longer contiguous with left liner portion 116 and right liner portion 118, respectively. As used herein the term “cavitation” does not refer to the generation of vacuum or a vapor phase as a result of sudden relative motion in a volatile liquid medium, but refers to the unwanted incursion of a fluid, either liquid or gas, into the interface between the Cup liner and the myocardial surface.

Bodily fluid or cavitated air has become entrained in such cavities 51 and 53. Such a condition is caused by one or more of the following: diastolic actuation and exposure of vacuum to the body, rapid or early application of vacuum by the extra-cardiac assist drive fluid on the heart 30; a poor fit of passive seal 113 to heart 30; sealing/blocking of port 111 by apex 38 of heart 30 in spite of the presence of drainage grooves 142 intended to prevent blockage of flow; or inadequate vacuum applied to vacuum port 111.

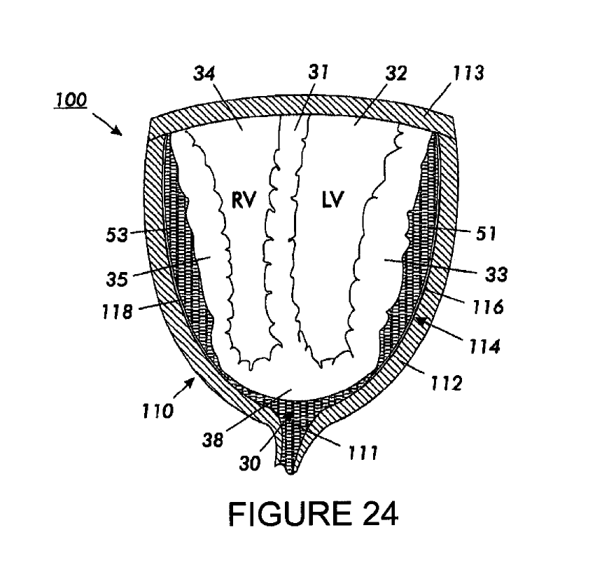

In such a situation, RV and LV filling are both compromised, as the extra-cardiac assist device separates from the heart 30 during diastolic actuation and the heart 30 fills passively and is not afforded diastolic assist. During systole, the heart is expelled from the confines of the housing 110 rather than the blood being expelled from within the ventricles 32 and 34. In instances where seal is lost over a number of cycles, substantially complete detachment of the heart 30 from wall 112 of the Cup shell 110 may occur, as depicted in FIG. 24.

It can be seen that apex 38 of heart 30 has become detached from vacuum port 111 of Cup 100. It is to be understood that the detachment shown in FIGS. 23 and 24 is depicted as a typical occurrence for baric-only actuating devices. The present invention includes an actively sealing rim located at 113 designed to prevent such failure modes.

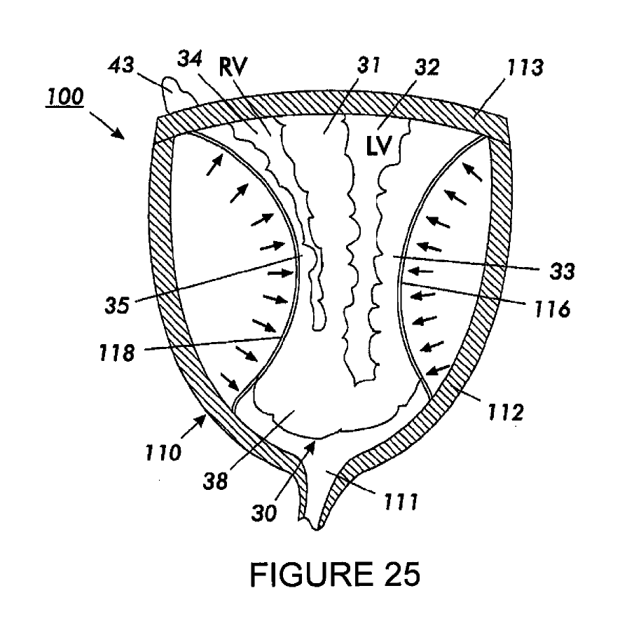

FIG. 25 depicts a situation wherein herniation has occurred during systolic actuation, this occurs despite adequate vacuum. The vacuum minimizes the space between the heart surface and the liner, but it does not prevent the heart from stretching axially. Since the heart is cone shaped, a slight axial displacement breaks the seal with the fixed position of the lip seal. Stretching the heart axially narrows the diameter over the entire length of the heart.

These are the two principal reasons for an active cup seal, one which can respond to changes in heart diameter, and preferably one which resists axial stretching such that the heart 30 is prevented from extruded from the physiologic extra-cardiac assist device Cup 100. Such stretching is a natural consequence of actuation fluid pressure during early systolic actuation and predominantly affects the RV infundibulum; i.e., the upper portion of the ventricle walls proximate to the atrio-ventricular (AV) groove and/or basal portion of the RV free wall.

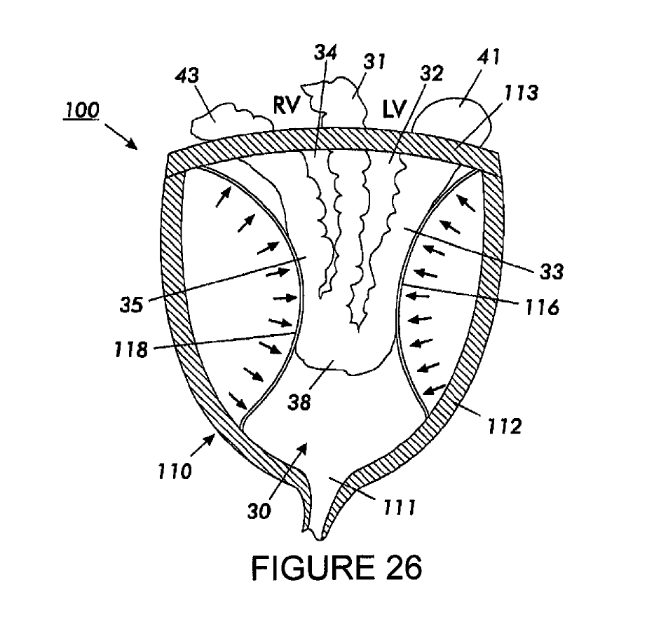

Referring to FIG. 25, where an active seal is absent, it can be seen that heart 30 has been forced into misalignment within Cup 100, and that an upper portion 43 of right ventricle wall (infundibulum or basal portion of the RV) 35 has been displaced upwardly beyond seal 113. In instances where the active seal is absent and such early systolic fluid pressure is sustained over a number of cycles, displacement of both ventricles 32 and 34 of the heart 30 from the Cup 100 may occur, as depicted in FIG. 26.

It can be seen that apex 38 of heart 30 has become detached with cavitation of air or fluid accumulation within the apical portion of the cup as the heart is displaced 111 from the Cup 100, and that upper portion 43 of right ventricle wall 35 and upper portion 41 of left ventricle wall 33 have been displaced beyond seal 113 of Cup 100. A combination of active lip seal 113 and anti-slip ring 120 prevent both axial and lateral slippage, one or both of which is responsible for the above described failure modes.

FIGS. 27-29 are cross-sectional schematic views depicting operations and/or effects of a physiologic extra-cardiac assist device on a heart afflicted with pulmonary hypertension and/or right ventricular hypertrophy.

Referring to FIG. 27, physiologic extra-cardiac assist device Cup 100 is depicted therein at the end of diastolic actuation. It can be seen that heart 60 afflicted with pulmonary hypertension (PHT) and/or RV hypertrophy is characterized in particular by a thickening of right ventricle wall 65, and the relative shapes of the septum 61, LV volume 62, left ventricular wall 63, and RV volume 64. The operation of physiologic extra-cardiac assist device Cup 100 can be programmed and/or controlled such that the energy rendered to heart 60 is a lower ratio, in terms of the ratio of inertial energy to baric energy, and is specifically matched to the needs thereof due to the PHT condition.

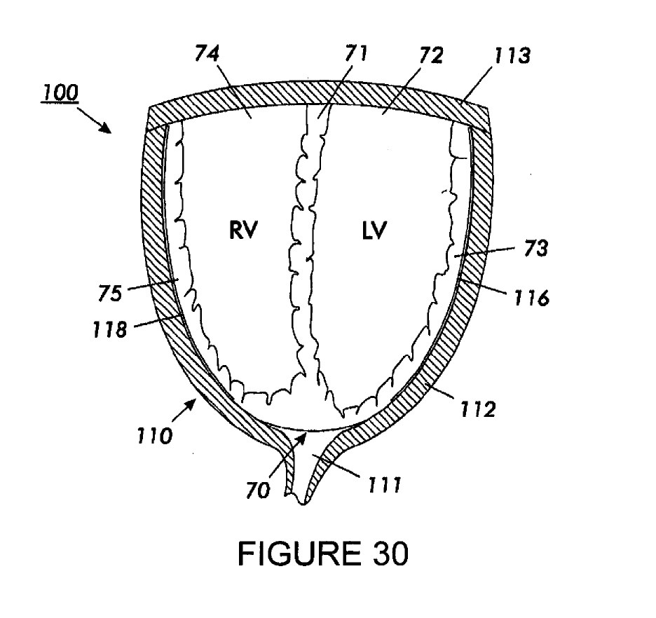

FIG. 30 is a cross-sectional schematic view depicting operations and/or effects of a physiologic extra-cardiac assist device on a heart afflicted with dilated cardiomyopathy. The condition of cardiomyopathy is in one sense the opposite of the condition of pulmonary hypertension since both walls of the ventricles are thinner than normal. Referring to FIG. 30, physiologic extra-cardiac assist device Cup 100 is depicted therein at the end of diastolic actuation. It can be seen that heart 70 afflicted with dilated cardiomyopathy (DCM) is characterized in particular by an overall dilation or enlargement of heart 70, accompanied by a thinning of left ventricle wall 73, right ventricle wall 75, and intraventricular septum 71, such that the volumes of left ventricle 72 and right ventricle 74 are increased. The operation of physiologic extra-cardiac assist device Cup 100 can be programmed and/or controlled such that the assistance rendered to heart 70 is specifically matched to the needs thereof due to the DCM condition. In particular, the inflation rate would be increased as far as possible in order to provide the largest ratio of inertial energy to baric energy.

In the present invention, the basic design of the Cup completely encompasses the heart from the atrio-ventricular groove (A-V groove) to the apex of the heart. Such a construction affords several advantages. A first advantage, enabled by liners of the present invention working with the Cup shell of the present invention, is the ability of the internal liner to compress or dilate the heart with a motion and force that is perpendicular to the heart tissue as previously described.

A second advantage of the Cup dynamic geometry of the present invention is the ability of the device to act and conform to both right and left ventricles in both systolic and diastolic assist, thereby supporting both pulmonary and systemic circulation.

A third advantage is the ability of the device to better maintain both right ventricle and left ventricle function to preserve flow balance.

A fourth advantage is the ability of the device to operate temporally and in terms of ejection fraction to allow the Frank-Starling mechanism to be functional in the healing cardiovascular system such that cardiovascular equilibrium is obtained.

A fifth advantage is the presence of active lip seal and slip resistant inner seal which together prevent abrasion and free wall bruising.

A sixth advantage is the freedom to use in combination a slightly larger cup volume compared to the patient's unhealthy heart volume with the active lip seal to treat a wider range of heart sizes with one Cup size and also provide for dynamic fitting during the course of therapy.

A seventh advantage is the perforated anchoring seam which fixes the liner to the Cup shell axially and approximately at the location of the septum such that inflation of the right side is delayed and this anchoring means used in conjunction with the two afore mentioned slip reduction seals so that actuation can be applied differentially to the left and right ventricles of a supported heart.

The Cup's dynamic geometry and the fluid drive control means of the physiologic extra-cardiac assist device of the present invention further provide for a full volumetric range of compression of the heart during systole, and a full range of volumetric expansion of the heart during diastole. The Cup's dynamic geometry and the fluid drive control means of the physiologic extra-cardiac assist device of the present invention further provide for a full temporal range of compression of the left ventricle Vs the right ventricle during systole.

This capability enables the physiologic extra-cardiac assist device to provide a full range of Systolic Pressure-Volume Relationships and Diastolic Pressure-Volume Relationships in a manner consistent with the Frank-Starling mechanism across a wide range of pathological conditions such that near normal cardiovascular function occurs and an equilibrium is reached which allows for a gradual transition from full support to device weaning and ultimate explant. The present invention also provides total circulatory support without direct blood contact, thereby decreasing the risk of thromboembolic complications including clotting, strokes, and other associated severe morbidity, and in some cases death, as well as significant blood cell lysis, which can adversely affect blood chemistry and patient health. This feature also eliminates the need for anti-coagulation drugs which reduces the risk for bleeding.

The present invention is a device that can be placed more rapidly than other existing devices from the start of the procedure, and therefore enables the unique ability to acutely provide life-sustaining resuscitative support, as well as continued short to long term support, as deemed necessary. All other cardiac assist device products (approved or in clinical trials) known to the applicants require surgical implantation with operative times that far exceed the ability of the body to survive without circulation. Physicians will welcome a device that can be placed when routine resuscitation measures are not effective. The number of failed resuscitations in the US annually is estimated to be on the order of hundreds of thousands. The device of the instant invention can support the circulation indefinitely as a means of bridge-to-recovery, bridging to other blood pumps, bridging to transplant, or long-term total circulatory support.

The present invention utilizes an active seal design that facilitates fitting a wide range of heart sizes to a single Cup size and further provides slip resistance and long-term reliability of the seal. Specific critical seal design features include the inflatability of the seal, thickness, shape, and durometer; and the location of the seal against the heart at the atrio-ventricular (AV) groove thereof. Additionally, one embodiment of the present invention utilizes a seal material that utilizes a swellable layer on the seal surface generally in contact with the heart.

Such swellable layers take in fluids from the surface of the heart, reducing its intrinsic lubricity as well as providing a tacky surface, a consequence of the fluid uptake, and also in this improved state of fit and fixation promotes the denaturation of proteins readily present and therefore affecting a proteinaceous bond between sealing surface and the surface of the heart. The infiltration of fibrin, which further improves the localization and long-term fixation of the heart relative to the liner surface by bonding to the liner over its entire inner surface, a condition much accelerate in time by localization of the heart within the Cup.

Embodiments of the present invention also utilize a liner coating material that promotes the controlled infiltration of fibrin, which further improves diastolic action and helps to minimize motion of the liner against the heart, which further minimizes abrasion between the liner and heart tissues. This additional coating is situated at least in an annular ring at the junction of the liner with the cup such that the heart is constrained against slippage.

Additionally, such coating may be applied in a strip aligned with the septum and approximately above the liner anchor which serves to separate left and right cavity portions and fix the liner proximal to the Cup shell inner surface, such that the heart is pulled taut in the plane of the septum by virtue of application of the apical vacuum which then constrains the septum against bulging into the RV volume. The remaining liner surface is smooth to provide for easy detachment after the fibrin bond has formed.

Alternatively, an elastic inner sheath placed between the liner and heart can be used to provide improve approximate fit and also comprise a porous surface specifically designed to provide fibrous ingrowth. The liner preferably is made of absorbable material such that the Cup can be detached from the bio-absorbable sheath with a minimum of trauma, and the liner once freed from the cup provides a minimum of loading to the healed heart and subsequently is digested by the body.

These and other novelties allow the physiologic extra-cardiac assist device Cup to be easily removed when the heart is in a condition of recuperation and the cardiovascular system is in a state of responsive equilibrium such that the Frank-Starling mechanism is restored so that the patient can be safely weaned off the device or is in a preferred state for bridge to another therapy.

In a further embodiment, the present invention also utilizes a liner or a secondary sheath that is biodegradable and/or one that becomes permanently attached to the heart's surface (with or without biodegradable properties) such that the physiologic extra-cardiac assist device can be removed by detaching the housing from the liner or sheath, leaving it in place. Such a liner can then instill favorable mechanical properties to the heart and/or provide drugs or other therapies (e.g., gene therapy etc., as described in greater detail elsewhere in this specification) but a secondary sheath will permit optimization of the liner for its mechanical properties and optimization of the secondary sheath for its biological and therapeutic drug properties.

Such therapeutic agents include but are not limited to anti-inflammatory agents, gene therapy agents, gene transfer agents, stem cells, chemo-attractants, cell regeneration agents, ventricular remodeling agents, anti-infection agents, tumor suppressants, tissue and/or cell engineering agents, imaging contrast agents, tissue staining agents, nutrients, and mixtures thereof. Such agents may be diffused or embedded throughout all or part of the liner, or alternatively, such agents may be contained within a gap formed within a liner comprising a first membrane in contact with the DMVA drive fluid, and a second membrane in contact with the heart, wherein the second membrane is permeable to the agent or agents. One or more portions of the liner may optionally be comprised of hydrophilic polymer capable of swelling a predetermined amount.

Thereby, the Cup serves a dual purpose of support of the heart for a period of time, and incorporating a therapeutic liner that is responsible for continued treatment of the underlying disorder. The liner can simply provide additional structural integrity through its mechanical properties, serve as a delivery agent, or a combination of both. Furthermore, the liner may simply be passive once the Cup is removed, but provides a simple, safe means of device detachment without otherwise risking bleeding or trauma to the heart that might result if it is removed.

In yet another embodiment, and in the case wherein the seal has been caused to be ingrown with myocardial tissue but the remainder of the liner is not ingrown with such tissue, removal of the liner is effected by separation from the seal. Thus only the seal will be left attached to the heart after Cup removal. Additionally this liner may provide reinforcement to a necrotic region of the heart associated with myocardial infarction, or it may provide a cushion and effective distribute the force of actuation when fresh bypass grafts are present.

Many existing cardiac assist devices, such as Left Ventricular Assist Devices (LVADs) require surgically perforating the cardiac chambers and/or major vessels. The present invention eliminates the need to perforate the heart or major vascular structures, and provides the ability to easily remove the device, leaving no damage to the heart and circulatory system once the heart heals and cardiac function is restored, or when the patient can safely be bridged to another therapy.

Existing cardiac assist devices, such as Left Ventricular Assist Devices (LVADs), which include axial flow pumps, produce blood flow that is non-physiologic and not representative of physiological pulsatile blood flow. The present invention avoids this condition and creates a near-normal physiological pulsatile blood flow both in terms of timing of the left and right ventricular pressures, in terms of stroke volume, in terms of flow balance, and in terms of end systolic pressures. Since blood passes through the natural chambers and valves of the native heart, which is more beneficial for vital end-organ function and/or resuscitation, particularly as it relates to restoring blood flow following a period of cardiac arrest or low blood flow the present invention is ideally suited to promoting the body's own cardiovascular regulatory and healing processes.

Furthermore, the present invention provides a physiologically compatible controllable environment surrounding the heart, which makes the operation of the physiologic extra-cardiac assist device system resistant to modes of operation that takes the patient's cardiovascular system out of normal equilibrium, thus it is better suited than extra-cardiac assist systems for recovering the natural heart. Alternatively, in the absence of natural recovery, can be used to apply pharmaceutical and tissue regeneration agents, even at localized concentrations that would not be tolerated systemically. This can be accomplished with or without use of a cup liner that is left on the heart following device removal, depending on the needs of the patient.

Furthermore, the present invention is able to augment heart function as is required to create and maintain required hemodynamic stability in a manner that is synchronized with the heart's native rhythm and in a manner that can alter the native rhythm toward a more favorable state. The purely complimentary nature of this support relieves the stress on the heart and promotes its healing.

As previously described, it is known that application of forces to the heart can cause potentially serious, irreversible damage to the heart by fatiguing, forcing the heart into an unnatural end-systolic shape and severely bruising the heart muscle, which can ultimately prevent it from functioning. The present invention avoids this very serious and potentially life-threatening condition by changing the shape of the heart in a physiologic way, by controlling the direction and type of forces applied to the heart and by controlling the magnitude and ratio of the difference energy forms applied to the heart.

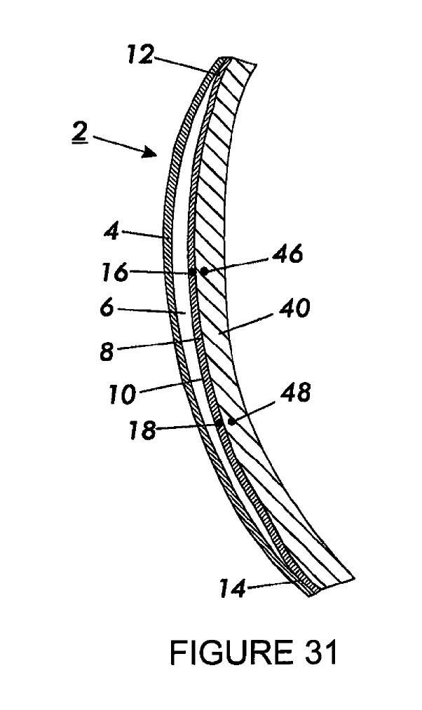

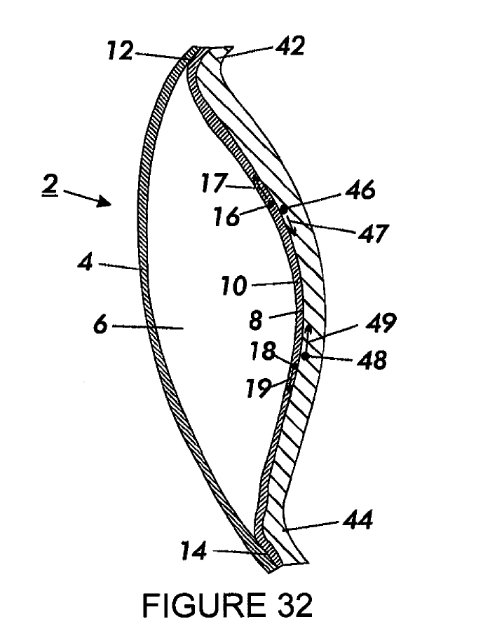

FIGS. 31 and 32 are cross-sectional schematic views depicting the action of a liner of a prior art extra-cardiac assist device upon the wall of the heart. Referring to FIGS. 31 and 32, in prior art extra-cardiac assist devices such as that disclosed in U.S. Pat. No. 5,119,804 and U.S. Pat. No. 7,494,459, there is provided an extra-cardiac assist device 2 comprising a rigid or dynamic shell wall 4 with passive sealing (in contrast to the present invention's dynamic shell wall, dynamic sealing characteristics and liner), and an elastic liner 10 joined to wall 4 at upper region 12 and lower region 14, thereby forming a cavity 6 between such liner and wall 4. The Cup and liner surround the heart, the ventricle wall 40 of which is contiguous with liner 10.

Again referring to FIGS. 31 and 32, in operation of prior art device 2, a fluid is pumped into cavity 6, thereby displacing liner 10 inwardly from shell wall 4. This displacement forces ventricle wall 40 inwardly a corresponding displacement, thereby resulting in systolic action of the heart.

However, it is noted that operation of the prior art device produces several effects that are undesirable. In FIG. 31 depicting the end-diastolic state of the device and heart, at the interstice 8 of liner 10 and ventricle wall 40, point 16 in the liner 10 and point 46 in the ventricle wall 40 are substantially contiguous with each other; and point 18 in the liner 10 and point 48 in the ventricle wall 40 are substantially contiguous with each other.

Subsequently it is apparent that in FIG. 32 depicting the systole state of the device and heart, at the interstice 8 of liner and ventricle wall 40, point 16 in the liner 10 and point 46 in the ventricle wall 40 have been displaced from each other as indicated by arrows 17 and 47; and point 18 in the liner 10 and point 48 in the ventricle wall 40 have also been displaced from each other as indicated by arrows 19 and 49.

This displacement is a consequence of several factors relating to the manner in which the liner 10 is joined to the shell wall 4 and to the properties of the liner material, which can produce localized non-uniformities in the stretching of the liner. The resulting displacement of point 16 and point 46 away from each other, and point 18 and point 48 away from each other produces localized shear stresses in these regions, which is very undesirable as previously indicated. In addition, such displacement also results in slippage of the liner along the surface of the ventricle wall, which over time can result in the undesirable abrading of the surface of the ventricle wall.

It is also known that there are shear stresses created along the circumferential direction of the ventricle wall, i.e., in the horizontal direction in the ventricle wall. Without wishing to be bound to any particular theory, applicants believe that these stresses are due to the tendency of the liners of prior art devices to self-subdivide during systolic action into nodes, wherein uniform portions of the liner are displaced inwardly, divided by narrow bands of the liner that are displaced outwardly.

In one embodiment described in U.S. Pat. No. 5,119,804, four such nodes are observed to be present when the device is operated without being fitted to a heart.

It is also apparent that regions 42 and 44 of ventricle wall 40, which are contiguous with upper region 12 and lower region 14 where elastic liner 10 is joined to wall 4, are subjected to intermittent high bending and shear stresses as a result of the repeating transitions between systolic and diastolic action of the device 2. Such intermittent bending and shear stresses can fatigue the heart tissue in these regions 42 and 44, and are thus clearly undesirable. These stresses occur also for the dynamic liner described in U.S. Pat. No. 7,494,459.

The reason for this is clear; in all these embodiments the liner is of more or less fixed surface area, and cannot follow in a one-to-one fashion the shrinking surface area of the epicardium. For these reasons, a sheath interposed between the liner and the heart is needed which is capable of reducing its surface area. Such layers are characterized as flowable in the sense they flow to form a new surface area. This condition can be achieved by either presenting a compressible layer with preferably close cell structure or a gel layer that grows in thickness and its surface area decreases.

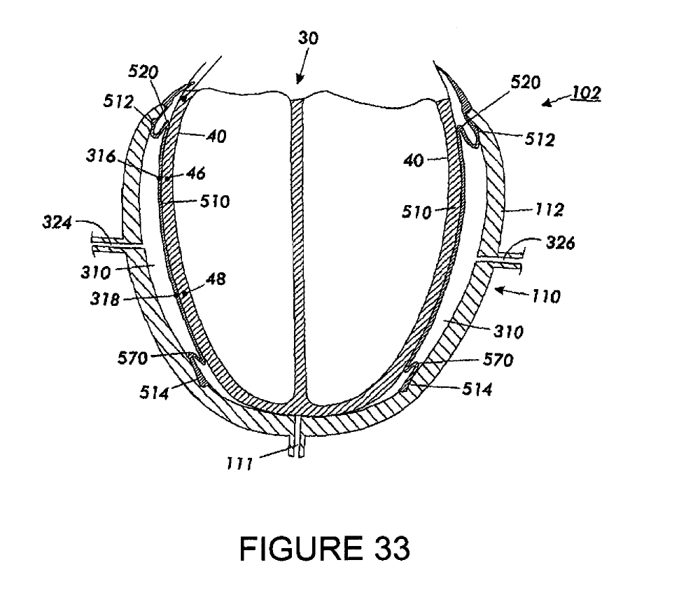

FIGS. 33-35 are cross-sectional schematic views depicting the action of the liner and sheath of the physiologic extra-cardiac assist device Cup of the present invention upon the wall of the heart. The liner need not cover the whole heart. The nodal structure is fixed by the placement of the anchoring lines anteriorly and posteriorly. Additional anchoring points may be needed for larger Cup sizes. Those additional to the anchoring lines separating left and right sides may be single point attachments so as not to impede flow within a right or left side. Such fixation is useful because it allows the inner linear to be as small in area as a relatively thin axial strip covering these nodal points.

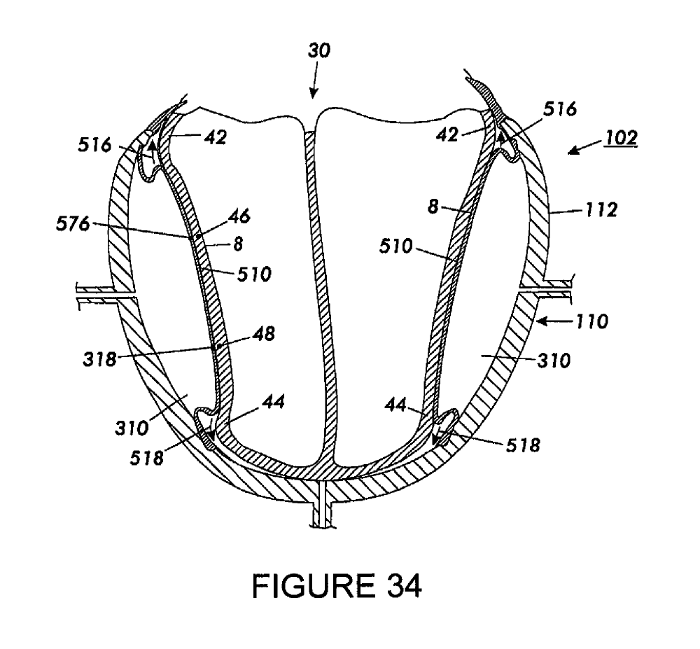

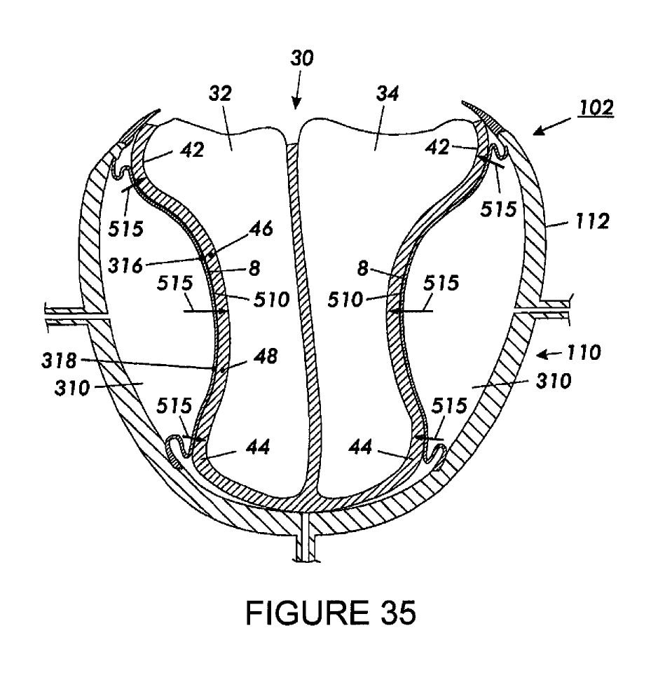

FIG. 33 depicts the diastole state of the device and the heart, FIG. 34 depicts the device assisting the systolic action of the heart at an intermediate stage of systolic action, and FIG. 35 depicts the completion of systolic action of the device and the heart.

For the sake of simplicity of illustration, the heart 30 of FIGS. 33-35 is shown with substantially thinner ventricle and septum walls than would typically be present in a physiologic extra-cardiac assist device assisted heart. Accordingly, there is no intent to limit the use of the physiologic extra-cardiac assist device to a heart of such proportions.

Referring to FIG. 33, physiologic extra-cardiac assist device 100 comprises a cup-shaped shell 110 having a rigid or semi-rigid wall 112, and a liner 510 joined at upper region 512 and lower region 514 to shell wall 112. Liner 510 joined to shell wall 112 thus forms a cavity 310 there between, into which a fluid is intermittently delivered and withdrawn. Such intermittent delivery and withdrawal of fluid to and from cavity 310 effects the cycling of the physiologic extra-cardiac assist device and the heart back and forth between the diastolic and systolic states.

In the preferred embodiment, liner 510 is provided with an upper telescoping liner section 520 and a lower telescoping liner section 570. Sections 520 and 570 may be comprised of one (a depicted) or more folds or sinusoidal corrugations which expand substantially laterally when inflated and collapse preferentially along the peaks and valleys of the corrugations. Interposed between the liner and the heart is a sheath 525, the surface area of which decreases and the thickness increases.

The effect of which is to cause points 316 and 318 within the liner to follow points 46 and 48 on the heart, and apply pressure (positive or negative) to the surface of the heart that substantially eliminates stresses tangent to the surface of the heart in cardiac tissue that otherwise such stresses result from the action of prior art devices previously described.

In operation, liner 510 is completely unloaded and the action of the working fluid on the heart is substantially normal to the wall 40 thereof. In other words, this embodiment of the present invention prevents the formation of substantial forces with components tangent to the heart wall within the heart muscle by applying forces to the heart that are perpendicular to the surface of the heart.

This embodiment allows the magnitude of the difference between adjacent forces to be large without introducing non-normal forces as is the case when a local inertial force is applied. In the case of inertial forces, the curvature of the liner is such that the forces are always normal to the heart surface, and non-normal forces cause the liner to flow to compensate. The use of such rolling diaphragm, as well as preferred liner materials to be subsequently described in this specification, eliminate the formation of shear forces within the heart muscle which leads to bruising damage to the heart tissue which in turn leads to muscle fatigue and potential failure of the heart. Thus the DMVA apparatus of the present invention is atraumatic, i.e., the apparatus does not inflict any injury upon the heart.

Rolling diaphragm sections 520 and 570 at the top and bottom of liner 510 are intended to reduce shear stresses in cardiac tissue, but without an interposed liner that can change surface area and preserve a one-to-one correspondence between points of the liner and points on the surface of the heart shear stress or motion will otherwise result from the action of the physiologic extra-cardiac assist device Cup 100.

Referring again to FIG. 33, the rolling diaphragm liner 510 comprised of upper rolling diaphragm section 520 and lower rolling diaphragm section 570 with one or more corrugations eliminates the single flexure regions of the liners used in earlier Cup designs. The angle through which any one fold travels from diastolic position to systolic position is distributed over the multiplicity of the folds and over the full length of the corrugated profile. Stress is therefore distributed rather than localized.

As was previously described and shown in FIGS. 31 and 32, such regions 12 and 14 of prior art device 2 where elastic liner 10 is joined to wall 4, are subjected to intermittent high bending and shear stresses as a result of the repeating transitions between systolic and diastolic action of such device 2.

Referring to FIG. 34, it can be seen that the displacement of the liner 510 by the filling of cavity 310 with fluid changes the radius of curvature of liner 510 with the center of curvature in the diastolic position at a location interior to the Cup to a radius of curvature of liner 510 with a center of curvature in the mid-systolic position at a location exterior to the Cup.

The curvature of liner 510 goes from a diastolic concave geometry to a systolic convex geometry. Consequently, the liner goes through a point in which the surface of liner 510 is approximately planar and its surface area is a minimum. However, the liner is essentially incompressible, and under the action of inflation is not inclined to compress in the plane of the liner. Thus there is need for a liner 525 that will compress in a plane tangent to the heart surface in regions where the liner is not anchored to the shell wall. Regions anchored to the shell wall do not go through a concave to convex transformation.

The consequence of this action is for the liner 510 to fold axially while the liner compresses circumferentially. This relationship between the liner and heart effects the systolic action of the heart without inducing substantial stresses in the ventricular wall 40 thereof. At the interstice 8 of liner 525 and ventricle wall 40, point 316 in liner 525 and point 46 in ventricle wall 40 have remained substantially contiguous with each other, and point 318 in liner 525 and point 48 in ventricle wall 40, as well as point 576 in the liner 524 and point 46 in the ventricle wall 40, have remained substantially contiguous with each other.

In addition it can be seen that the radius of curvature in upper region 42 and lower region 44 of ventricle wall 40 is substantially greater than such radius of curvature resulting from the use of the prior art device as depicted in FIG. 34.

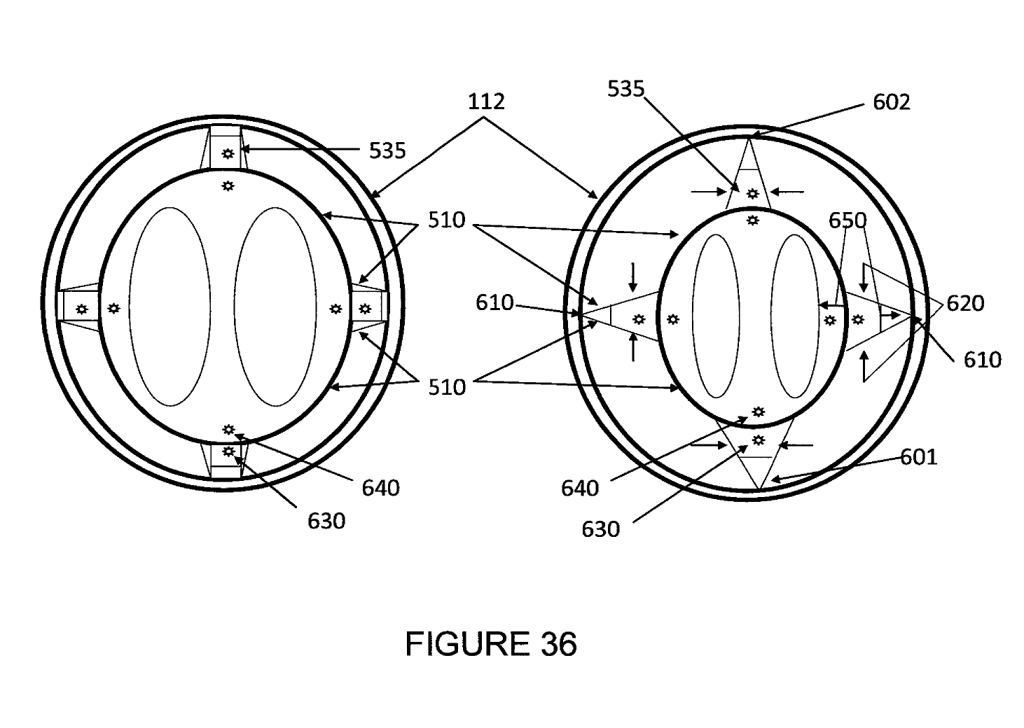

Thus, the bending stresses produced in regions 42 and 44 of ventricular wall 40 are substantially less as a result of the use of liner 525 of the present invention. It can be further seen that liner 525 is engaged with ventricle wall 40 in a progressing compressive action as indicated by upper arrows 516 and lower arrows 518. The matter of axial folding will be addressed in FIG. 36.

FIG. 35 is a cross-sectional view depicting the physiologic extra-cardiac assist device apparatus assisting a heart, at the completion of systolic action of the device and the heart.

Referring to FIG. 35, the displacement of liner 510 of apparatus 102 is at its maximum value, having squeezed ventricular walls 8 to an optimal conformational change wherein heart 30 has an approximately “hour-glass” or “apple-core” shape, with a minimum diameter, (i.e. the “cardiac core diameter”) at the plane defined by opposing arrows 515. At the completion of systole, apparatus 100 has caused, or assisted in the displacement of, a cardiac ejection fraction of approximately 0.55 from left ventricle 32 and right ventricle 34. At this position it can be seen liner 510 is in a convex geometry, and liner 525 is once again in a relaxed configuration similar to the end diastolic position of FIG. 33. The liner 525 has transformed from relaxed end diastolic state to maximally compressed mid systolic state, to relaxed end systolic state. Throughout this transformation points on the heart and points on the liner 525 have remained juxtaposed.

Even at the maximum displacement of liner 510, it can be seen that at the interstice 8 of liner 510 and ventricle wall 40, point 316 in liner 525 and point 46 in ventricle wall 40 have remained substantially contiguous with each other, and point 318 in liner 535 and point 48 in ventricle wall 40 have remained substantially contiguous with each other; and that the radius of curvature in upper region 42 and lower region 44 of ventricle wall 40 is substantially greater than such radius of curvature resulting from the use of the prior art device as depicted in FIG. 32. Thus, the bending stresses produced in regions 42 and 44 of ventricular wall 40 are maintained at a low value.

Referring again to FIGS. 34 and 35, it can also be seen that liner 510 has rolled progressively as indicated by arrows 516 and 518, to a maximum extent along upper ventricle regions 42 and lower ventricle regions 44 shown in FIG. 35. The force applied by liner 510 upon ventricle walls 40 at all points along interstice 8, resulting from the isotropy of the fluid pressure within cavity 310 during end systole, is substantially perpendicular to ventricle walls 40, as indicated by arrows 515. During this action liner 525 is compressed progressively in the regions 515 until reaching a maximum compression at end systole. Thus, the presence of any shear force in the ventricle walls 40 in this region is minimized.

FIG. 36 is a cross sectional view perpendicular to the axis of the heart. The liner 510 is in the end systolic position as also depicted in FIG. 35 in cross sectional view parallel to the axis of the heart. Note nodal folds oriented axially and corresponding to anterior 601 and posterior 602 anchoring lines and nodal folds 610 corresponding to single point anchors located circumferentially midway across of ventricular free walls. Arrows 620 indicate the local vectors of liner 535 which serve to keep liner points 630 in juxtaposition with ventricular points 640. Note during systolic actuation the width of liner strip 535 narrows (not drawn to scale) in response to force vectors 620 and the width increases as indicated by vectors 650. The same principle holds if the entire inner surface of the liner was covered with a compressible sheath. Additionally, liners 535 can be swellable such that proteinaceous fluid present in the Cup caused the liner elements to affix to the myocardial surface and thus provide lateral and axial fixation.

Referring again to FIG. 33, wherein physiologic extra-cardiac assist device apparatus 102 is provided with a further innovation with a first physiologic extra-cardiac assist device drive fluid port 324 and a second physiologic extra-cardiac assist device drive fluid port 326.

In one embodiment, the portion of cavity 310 that is in communication with drive fluid port 324 is made separate from the portion of cavity 310 that is in communication with drive fluid port 326 by making the anterior 601 and posterior 602 anchoring lines nonporous and spanning the distance from lip seam to apical seam of the liner. In addition, each of ports 324 and 326 are provided with separate physiologic extra-cardiac assist device fluid supply/withdrawal means. In this manner, the fluid cavity in communication with drive fluid port 324 can be filled and emptied independently of the fluid cavity in communication with drive fluid port 326, so that right ventricle 32 (see FIG. 14) can be actuated independently of left ventricle 34.

An alternative approach to adjusting left-right balance may be based on one or more physical barriers to the flow of drive fluid from its first (LV) entry position to the opposite (RV) side of the heart.

One example (illustrated in FIG. 43 and discussed later) is a pair of flow limiting partitions aligned approximately at the septal plane, and protruding inwardly from the physiologic extra-cardiac assist device Cup shell or outwardly from the physiologic extra-cardiac assist device Cup liner, or both. These partitions may alternatively be attached to both shell and liner at the septal plane, and be porous to the degree that they delay pressure rise on the RV side of the Cup while not overly impeding the flow of drive fluid (typically air).

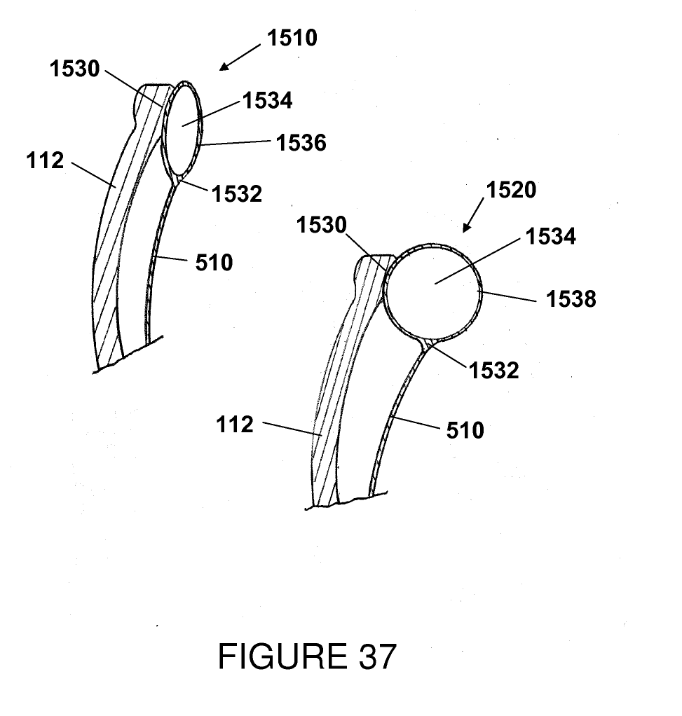

FIG. 37 depicts an inflatable rim seal used to affix the physiologic extra-cardiac assist device Cup on the heart and to provide an effective seal to prevent excess gas or fluid from entering the space between the physiologic extra-cardiac assist device Cup liner and the surface of the heart. The rim seal is hermetically bonded to the body of the physiologic extra-cardiac assist device Cup and preferably may be otherwise coated with a temporary bio-adhesive as described elsewhere in this description. The left side of FIG. 37 shows the rim seal in its initial un-expanded state and the right side of the figure depicts the rim seal partially inflated. A separate lumen (not shown) connects the interior of the rim seal to an external pump and valve. It will be obvious to those skilled in the art that a variety of fluids and designs may be used to effect the proper actuation of the rim seal.

It will readily be seen that by incorporating the rim seal in the design of the physiologic extra-cardiac assist device Cup a relatively large range of heart size may be accommodated in a single Cup size. Thus, a smaller number of manufactured Cup sizes will be required, and the process of sizing a Cup to a specific patient is made far simpler. In addition, when the rim seal is deflated, the process of placing the Cup over the heart during implantation is made easier; when removing the Cup from the heart, deflating the rim seal makes removal easier.

The bio-adhesive may be any of a variety of biocompatible and preferably bio-absorbable materials known to those skilled in the art. In particular materials that are tacky and hydrophilic are preferred; these may be applied as a thin coating but may also be applied in a relatively thick layer of 1 millimeter or more, and upon contact with body fluids may swell to multiples of this thickness.

In combination with the features shown in FIG. 37 an additional free liner or sheath (not shown) may be fitted to the interior of the physiologic extra-cardiac assist device Cup in order to improve fit of the Cup to a particular heart. The liner is preferably formed from a relatively soft polymer having modulus at or below 25 Shore A. The liner may also be formed from a closed cell foam material, or alternatively from an open cell foam material that serves to draw body fluid by capillary action, or conduct retention vacuum across the surface of the heart, or both. Changes in the aggregate property of the open cell material as it absorbs and reacts with entrained body fluids may be designed to increase its Poisson ratio to 0.5 or higher over a time period ranging from minutes to hours after implantation of the physiologic extra-cardiac assist device Cup.

An additional feature that may be incorporated into the physiologic extra-cardiac assist device Cup is a semi-rigid patch that protects a particularly vulnerable area of myocardium from the forces associated with support by the physiologic extra-cardiac assist device Cup. This patch is preferably affixed to the liner at the time of surgical installation of the Cup so as to be properly aligned with the friable area of tissue.

Referring to FIG. 37, two views show the relative shape of a Cup with its annular retention ring deflated 1510 and also with its annular ring fully inflated 1520. The annular ring 1536, 1538 is affixed to the shell of the Cup 112 with an appropriate adhesive at location 1530. The interior 1534 of the retention ring may be filled with air or another gas, with a liquid, a gel, or a material such as liquid silicone rubber that cures over time after inflation. Each of these approaches may have its benefits in terms of rigidity, physical stability and fatigue resistance, and the ability to deflate the ring for ease of explantation. Means for inflating and deflating are not shown but a simple approach would be a small tube connecting the retaining ring with an external supply of the desired material. This tube may be integrated with the shell 112 at time of manufacture, or alternatively may be retained along the exterior surface of the shell.

The junction 1532 between liner 510 and retention ring 1536, 1538 describes a circle not shown in the cross-sectional view, and its tapered shape in transitioning between the ring and liner is intended to minimize material fatigue caused by cyclic bending during operation. The liner 510 in both views 1510 and 1520 is shown in FIG. 37 in a relatively full diastolic position; it will be appreciated that cycling between diastolic and systolic shapes will result in highly repetitive bending of the liner 510 and junction 1532, so the tapered shape is intended as a stress relief. The relative shape of the inflatable retention ring 1536, 1538 and the location of the junction 1532 between the ring and liner 510 is also carefully designed so that the shape of the interior of the Cup assembly will track the natural shape of a heart as the annular ring is adjusted to a particular patient's physiology.

The design of the physiologic extra-cardiac assist device Cup is carefully balanced to provide several features effective operation on hearts that span a large range in sizes; effective retention on the heart during operation provided in part by vacuum applied at the apex of the Cup, but also on retention provided by the inflatable annular ring and by bio-adhesive applied in the region of the liner and ring near the A-V groove; ease of installation and removal of the Cup provided by deflation of the retention ring; and shape factors that accomplish the above without creating a pinching action in the area near the A-V groove.

A more detailed description of the invention, which is a method for using internally sensed data in conjunction with extra-cardiac assist devices, is now presented.

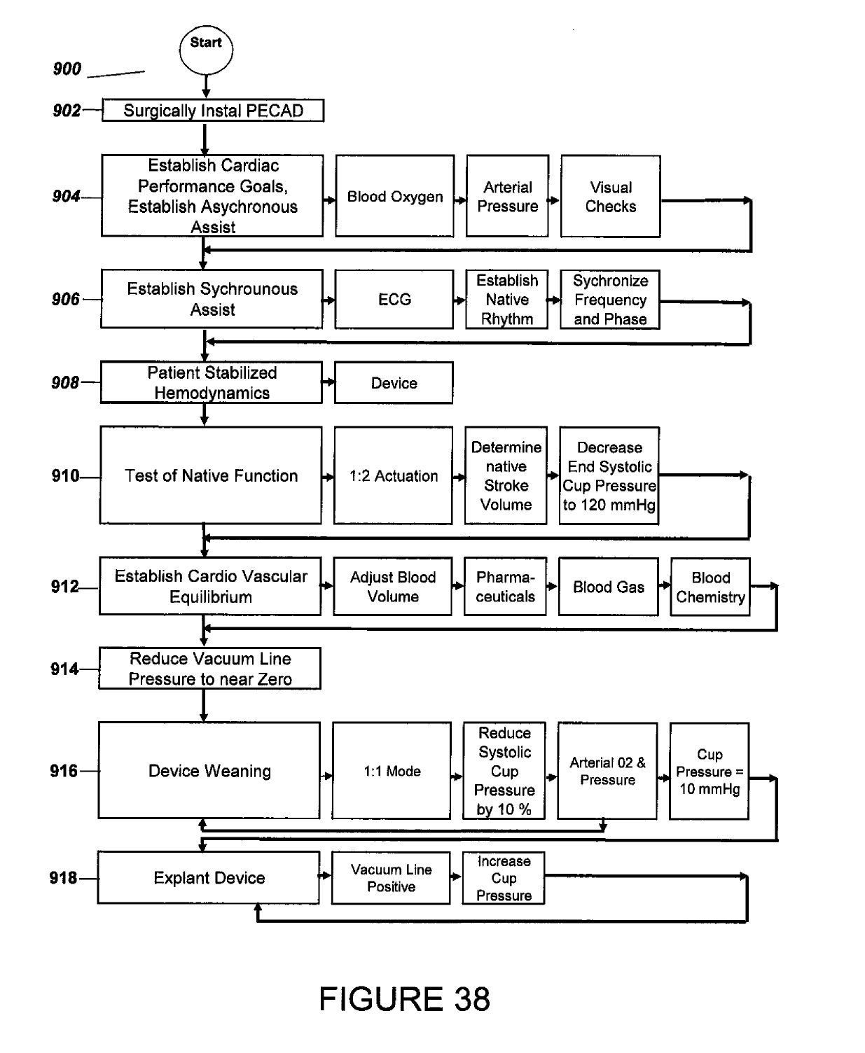

FIG. 38 is a flow chart depicting such a method for using internally data to guide physiologic extra-cardiac assist device installation in an emergency situation and to assess cardiac performance under the influence of physiologic extra-cardiac assist device. Referring to FIG. 38, method 900 includes the following steps 902-918, which are offered here as illustrative and not limiting.

It is presumed that the patient's heart has stopped beating or is beating insufficiently to sustain life. Therefore at this initial point there is no need for synchronizing the actuation of the device to an electrocardiogram.

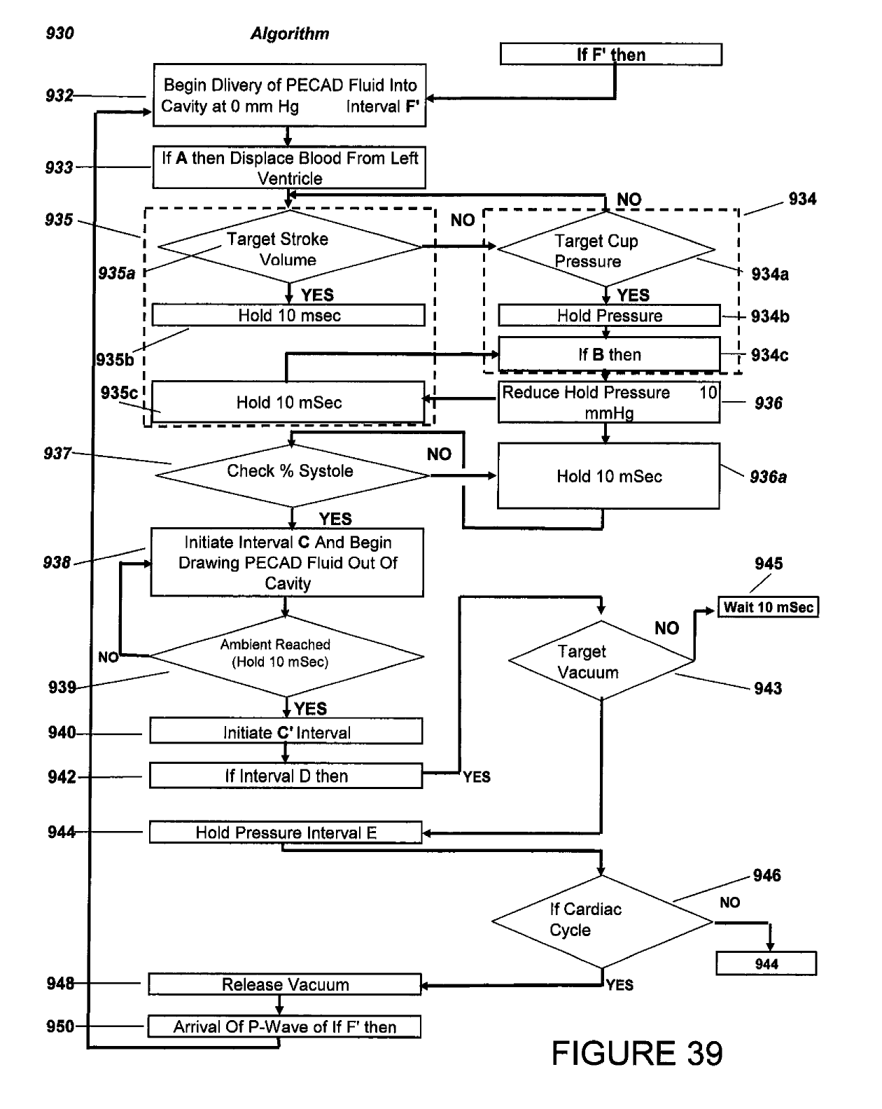

In step 902, the physiologic extra-cardiac assist device Cup is placed on the exposed heart using suction, the adjustable rim seal is inflated until no fluid is flowing in the vacuum line and the physiologic extra-cardiac assist device Cup is firmly held in place at the A-V grove, and the device is actuated using the asynchronous algorithm detailed in FIG. 39. Improvement in the patient's cardiovascular status is assessed by traditional diagnostic means.

For example, arterial blood pressure and arterial oxygen saturation. The patient should be placed on a heart monitor, if not already on one, to assess the patient's electrical status. Subsequently, in step 904 required performance improvement objectives are established. In step 904, patient's blood oxygen and arterial pressure is monitored as well as visual checks of the retention of the heart within the Cup. The vacuum and/or basal rim seal pressure can be increased or decreased to improve the localization of the heart within the Cup. Venous pressure or visual inspection can be used to assess left-right flow balance. Increasing the ratio of left flow to right flow can achieved by increasing the inertial to baric energy ratio, as described in detail subsequently. Once pressure and blood oxygen are shown to be life sustaining and stable, attempts can be made to restore heart rhythm by electrical means.

Step 906 involves transition from asynchronous assist to synchronous assist. When regular ECG is restored, the external ECG leads are connected to the physiologic extra-cardiac assist device. The system will detect the presence of a regular ECG and activate the synchronous activation button and indicate the system is ready. Depression of the synchronous button causes the system to slowly adjust frequency and phase of actuation to coincide with the native heart rhythm. The system will show on monitor concurrent traces of ECG and cup actuation. The clinician may adjust the delay between EC and cup actuation using visual cues obtained by watching the motion of the heart within the cup, as well as other physiologic data. Blood oxygen can generally be obtained in real time and is of particular use in these fine adjustments. Other adjustments include vacuum level, end systolic pressure, and % systole, as described later in detail.

In step 908, the patient is stabilized, optionally the chest is closed and the heart is well localized within the cup. At this point the clinician may want to assess the need for additional support.

In step 910, the clinician selects the 1:2 button which causes the system to actuate the cup on every other beat and open the liner cavity to ambient on every beat not actuated. If the native heart has no or little systolic function the heart will become congested during the no-beat cycle, and during the next actuation the cardiac output will generally be 1.5 to 2 times the normal stroke volume due to this congested state.

Accordingly, the patient generally suffers no decrease in mean blood flow when placed in the 1:2 mode for limited periods of time. The system monitors changes in cup volume using flows and pressures during actuation, and during the off cycle. During the off cycle, if the heart is contracting air will be drawn into the liner cavity. In this mode a real-time plot of cup volume during and off actuation is plotted. With this display the clinician can assess if the native contractility is improving or worsening. In an improving scenario, the clinician may elect to use a higher value for end systolic pressure during the support periods to unload the heart during the off period. As the intensity of the native contractions improves, the systolic support pressure can be decreased to approximately 120 mmHg.

In step 912, the clinician is primarily concerned with establishing cardiovascular equilibrium. For example, the mean arterial pressure should be brought to a normal level by adjusting fluids, vasodilators, or vasoconstrictors. All blood gases and blood chemistry should be near normal. Distal perfusion of organs and their function should be improving. At this point it should be clear whether the patient will require a long term support device or is likely to recover without additional mechanical support.

In step 914, if a liner is used generally the heart is well localized within the cup within a couple of hours. The clinician can reduce the vacuum line negative pressure in successive steps, watching for heart motion out of the cup or evidence of rim seal insufficiency. The presence of fluid flow in the vacuum line indicates poor seal. If the seal is maintained in most cases the vacuum can be reduced to 0, or a minimal maintenance level. At this point cardiovascular equilibrium is maintained during a recovery period that may span hour, days, or longer.

In step 916, in the case where the patient is likely to recover the clinician then starts the device weaning process; this is dependent on a number of factors related to patient condition so its time span may be minutes, hours, or even days. This process is designed to gradually reduce support while actuating the cup in a way that does not load the heart. In this mode, the patient is generally driven in the 1:1 mode and the support level is reduced and applied uniformly for every heart beat. Generally, weaning consists in reducing end systolic pressure to an end point of 10 mmHg above ambient. In a later phase, the diastolic support is reduced to approximately −10 mmHg below ambient. When adequate blood oxygen and pressure is maintained under these conditions, the patient is ready for device explant.

The process of weaning may involve the following steps of (a) actuating the heart in synchrony with native ventricular contraction, (b) actuating the heart on every other cardiac cycle, (c) assessing the health of the heart during the non-assist cycles by measuring change in cup volume while the driveline is freely open to the atmosphere, (d) measuring the change in cup volume during the assist cycles, (e) determining a measure of heart health by comparing the changes in cup volume during assist cycles and during non-assist cycles, (f) gradually reducing the amount of support energy until the heart is beat substantially on its own, optionally when the ratio of volumes computed in step (e) reaches a value less than 2.0, (g) releasing the seal around the base of the heart, (h) making the suction line slightly positive pressure, and/or (i) gradually increasing the support level until the heart decouples from the device.

In step 918, device explantation is conducted. In the case where an absorbable liner is placed between the cup and heart, the goal is to detach the cup from the liner. In the case where the liner is attached to the cup or no liner is used, the goal is to detach the heart from the cup. The proteinaceous bonds to the cup are not strong and fail readily in a peal mode. First the vacuum line pressure is adjusted to be slightly positive, but not greater than the end systolic drive pressure. Then the adjustable rim seal is evacuated to its most open position. Following this the end systolic pressure is gradually increased until the heart will be ejected from the cup and the cup can be removed. Additional increases of pressure in the vacuum line can be made as the end systolic pressure is increased, this will cause the release of the heart from the cup.

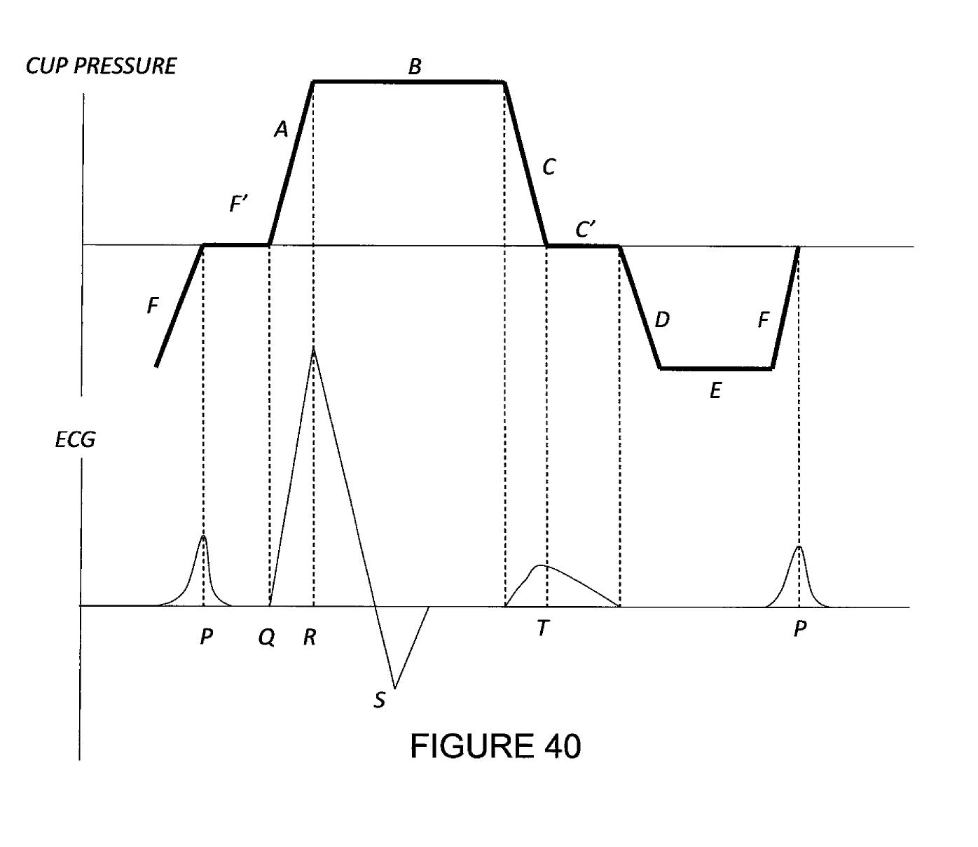

More detailed descriptions of the invention, which is directed to methods and algorithms for specific feedback control of the physiologic extra-cardiac assist device Cup, are now presented, with reference in particular to FIGS. 39 and 40.

FIG. 39 is a flowchart of one specific algorithm for automatically adjusting the function of an embodiment of the physiologic extra-cardiac assist device Cup. It is to be understood that this algorithm is one example of many that are possible, which may be defined and selected according to the particular patient and cardiac disorder for which physiologic extra-cardiac assist device assistance is indicated. For a better understanding of the following description of method 930 of FIG. 39, reference may also be added to algorithm 960 FIG. 40 and FIGS. 13 and 14-30, the latter were previously described in this specification. It is to be understood that pressures provide in millimeters of mercury (Hg) are gage pressures, with 0 mm Hg being ambient atmospheric pressure.

Referring to FIG. 39 and FIG. 16 and the A-F algorithm 960 of FIG. 40, method 930 begins at the initiation of Interval F′, ahead of the heart systole and corresponding to the P-wave of the ECG with step 932, wherein delivery of drive fluid into cavity 119 of physiologic extra-cardiac assist device Cup 100 begins, at a delivery pressure of 0 mm Hg (ambient). This interval is not an actuation interval but is needed when a single driveline powers the Cup. In order to make optimal use of the reservoir pressure it is desirable to equilibrate the driveline to ambient before supplying positive drive pressure.

In step 933, Interval A is initiated at the Q-wave, blood begins to displace from the left ventricle 32. Cup pressure is calculated from driveline pressure, enabling check 934. Blood volume and/or flow sensors, and imaging and/or other cardiac state sensors described elsewhere in this specification optionally provide data to the physiologic extra-cardiac assist device controller; more typically the volume supplied to the cup is calculated internally using an equation involving flow, time, and pressure, enabling check 935.

If the Cup volume is twice the target stroke volume the drive pressure is decreased 10 mmHg and the Cup volume rechecked. The system internally checks coincidence of the hold pressure with the occurrence of the R-wave. The reservoir pressure is increased 10 mmHg in the next cycle if the target volume occurs after the R-wave and decreased 10 mmHg if the target volume occurs before the R-wave.

During Interval B, step 936, and after the hold pressure has been maintained for at least 10 ms step 937 checks the elapsed time of combined Intervals A and B and divides this time to the duration of the last cardiac cycle and calculates a % systole value (Interval A&B/Interval A-F). If no % systole value is input from the console, Interval C is initiated. If a value for % systole has been input and it is equal to or greater than the input value Interval C 940 is initiated.

Step 937 switches the driveline from the pressure reservoir to the vacuum reservoir and begins drawing fluid out of physiologic extra-cardiac assist device Cup. Check 939 is made of the driveline pressure, when it reaches ambient this pressure is held. If the ambient pressure is held for more than 10 ms then the coincidence of hold ambient and the start of the T-wave is compared. If a value for % systole is input, then the algorithm proceeds to step 942. If no value for % systole is input, then ambient is held to completion of the T-wave.

If the decision to go to step 942 occurs after completion of the T-wave, then negative pressure of the vacuum reservoir is increased by 10 mmHg.

Step 942 reconnects the vacuum reservoir to the driveline. Check step 943 measures the pressure in the driveline and compares to target vacuum pressure. If the target pressure is held for 10 ms then Interval E is initiated in step 944. Check step 946 compares the total elapsed time from Interval A to the current time and compares this with input cardiac cycle (in asynchronous mode) or prior measured time period from cardiac cycle P-wave to P-wave (in synchronous mode). If current elapsed time is equal to cardiac cycle minus 50 milliseconds then step 948 is initiated. During interval F, step 948 passively equilibrates the driveline to ambient. In the synchronous mode, arrival of the P-wave triggers step 950 which returns the control process to step 932, which uses pressure to force the driveline pressure to ambient, as needed.

Alternatively, in asynchronous mode, step 950 is triggered by the cardiac cycle input value.

The algorithm and method of FIGS. 39 and 40 are predicated on the use of a plenum driven system in which reservoirs of pressure and vacuum are switched by servo valve to the driveline. In a piston driven system intervals C′ and F′ are not needed since there is no possibility that pressure will be delivered discontinuously to an evacuated driveline and vice versa.

A more detailed description of the invention, which is directed to internal sensing types and methodology, is now presented with reference to FIGS. 41-49.

Cup volume is measured at all times relative to ambient pressure. The calculation involves resetting the volume value during intervals C′ and F′, during which time the cup volume is presumed to be zero. Increased cup volume is calculated by integrating the value of instantaneous flow X pressure/target pressure. This value is used by the internal controls to compare with input value heart stroke volume or cardiac output. In the case of input value for cardiac output, the cardiac output is divided by input heart rate or ECG determined heart rate and multiplied by 2 and compared to measured cup volume.

In the case of input value for stroke volume, this value is multiplied by 2 and compared to measured cup volume. Driveline pressure and flow can be measured within the console in the first length of tubing.

This control method is not dependent upon measuring ECG, but in instances where ECG is available for input, simple AC coupling with derivatives applied to the waveform is suitable for determining the various wave structures. Alternatively, the output waveform could be display and the clinician could assign the various interval markets, and the system could use the relative positions of these markers in time and the easily detectable QRS peak to provide for real-time synchronization. The ECG signals used in timing the system are preferably derived from standard external three-point monitoring schemes.

The purpose of physiologic extra-cardiac assist device is to maintain cardiac output. This output may be characterized by stroke volume (the volume of blood expelled from the heart during each systolic interval) and pressure at which this volume is delivered from the heart. In yet another embodiment of the present invention, working fluid pressure, and/or flow rate sensors are integrated into the Cup and/or Cup drive assembly to collect data that can be used to control the inflation/deflation of Cup liner, which in turn enables control of stroke volume and blood pressure.

In more elaborate but more precise application of the fluid volume and pressure sensing, FIG. 41 is a schematic representation of working fluid pressure and/or flow rate sensors integrated into the Cup and the drive assembly thereof.

Referring to FIG. 41 physiologic extra-cardiac assist device Cup 108 comprises fluid pressure sensors 1261, 1263, 1265, and 1267, which are placed between the Cup shell 110 and liner 114 (pressure sensor 1261), and/or within the liner inflation/deflation duct 322 (pressure sensors 1263 and 1267), and/or within the pump assembly 330 (pressure sensor 1265) used to pump physiologic extra-cardiac assist device working fluid indicated by arrows 399 from within physiologic extra-cardiac assist device control unit 1301. By measuring the pressure of DMVA working fluid over time it is possible to infer the volume of working fluid delivered to Cup 108.

Alternately, the volume of working fluid delivered to Cup 108 can be measured directly by placing flow rate sensor(s) 1269 within liner inflation/deflation duct 322 to measure the rate of flow of working fluid into or out of Cup 108 as indicated by arrows 399. Alternately, the flow of working fluid into Cup 108 can be determined by calculating the volumetric displacement of pump 330.

In one embodiment wherein pump assembly 330 of physiologic extra-cardiac assist device 108 comprises a piston pump, such volumetric displacement is determined by multiplying the cross-sectional area of the bore 332 of pump cylinder 332 or of pump piston 334 by pump stroke 336 due to piston driver 338. It is to be understood that similar means can be used to determine volumetric displacement of other types of fluid pumping devices.

Sensor output from sensors 1261, 1263, 1265, and 1267, and/or other sensors described previously or subsequently in this specification, is delivered to the physiologic extra-cardiac assist device control unit 1301, which in turn directs the inflation and deflation of the Cup liner 114 as required to provide the desired amount of cardiac output. Information will be used to optimize physiologic extra-cardiac assist device action on the heart, dictate weaning protocols and algorithms, etc. In another embodiment, fluid flow rate sensors monitor the inflation and deflation volume of the liner(s), which correspond respectively to the systolic output from and diastolic input to the heart. By controlling the total volume of fluid pumped into and out of the liner(s), the physiologic extra-cardiac assist device is able to precisely control stroke volume.

In other embodiments, blood pressure is controlled in a number of ways, including the use of Cup working fluid flow rate sensors. The vascular structure of the body has a variable resistance to blood flow as the body opens and closes resistance vessels depending upon a variety of internal and external factors.

Typically, resistance does not change much in a minute. However, a sudden change such as e.g., a precipitous decrease in ambient temperature will produce a very rapid change in resistance, due to such factors as the diameter, length, and geometry of arteries, veins, etc., which restrict the flow of blood. Therefore increasing or decreasing the rate of Cup liners inflation against this hemodynamic resistance will either increase or decrease systolic blood pressure, respectively.

Likewise, increasing or decreasing the rate of Cup liner deflation against this hemodynamic resistance will either increase or decrease diastolic blood pressure, respectively. Since the rate of flow of working fluid into the Cup liner directly controls liner inflation and deflation, measurement and control of Cup working fluid flow rate sensors can also be used to control blood pressure. In yet another preferred embodiment, the Cup working fluid consists essentially of an electro-rheological fluid (e.g., isotonic saline) that provides a unique and easily detectable flow rate signature.

In another embodiment, blood pressure is controlled by use of Cup working fluid pressure sensors. Since Cup liner inflation or deflation is dependent upon the pressure at which the working fluid is delivered to or removed from the liners, it is possible to use measurement and control of physiologic extra-cardiac assist device working fluid pressure to control blood pressure. Specifically, the higher or lower Cup liner inflation or deflation pressures can be used to control systolic or diastolic blood pressure, respectively.

Any of the aforementioned control methods and algorithms can additionally employ means to control the type of energy form delivered to the heart. The primary reason for this is to control left-right cardiac output characteristics.

The following properties of the Cup and physiologic extra-cardiac assist device method as described in this disclosure are critical to obtaining left-right cardiac output control inertial actuation is local; 70% of pumpable blood volume is located in 25% of heart length; the heart is constrained against left-right motion and axial motion; a seal with active compliant adjustment is needed to reduce long term exposure of heart to vacuum; adjustment to heart size with an inflatable ring seal is needed to provide custom sizing to a wider range of heart sizes; and/or the ability to wean the heart off physiologic extra-cardiac assist device is conditional on the ability of the heart/device system to be able to reproduce Frank-Starling mechanism.

Blood flow kinematics associated with the pumping ventricle of the heart obeys the following equation:

W=PV+½mv²+kx+max

Where W is the work performed on the heart by a physiologic extra-cardiac assist device, P is the mean change in pressure, V is the mean change in volume, m is mass, v is the mean change in velocity, k is a spring constant, x is a displaced distance, a is the mean acceleration. More precisely we have:

$${W}_{baric} = - {\int }_{{v}_{i}}^{{v}_{f}} P d V$$

Which expresses the baric work done on or by the heart for a given pressure and change of volume Vf−Vi.

$${W}_{inertial} = {\int }_{{p}_{i}}^{{p}_{f}} p d p = 1 / 2 {mv}^{2}$$

Which expresses the inertial work done on or by the heart for a given change in momentum.

$${W}_{potential} = {\int }_{{x}_{i}}^{{x}_{f}} k d x = kx$$

Which expresses the potential work done on or by the heart for a given change in potential energy. There are strategies for increasing the potential energy composition of a delivered amount of support energy. Potential energy is stored in the walls of the heart and more importantly in the walls of the major vessels proximal to the heart. Once the valve closes, the potential energy stored in the aorta is an important source of energy that sustains arterial flow during the diastolic phase of the cardiac cycle.

The term max is the time derivative of the inertial term; we will call the accelerative term.

W_{accelerative}=mx(dv/dt)=max

Which is damped out fairly quickly and converts primarily to inertial and potential energy. Thus we have the general equation for work:

W(P,x,\dot{x},\ddot{x})=W_{baric}(P)+Wₚₒₜₑₙₜᵢₐₗ(x)+Wᵢₙₑᵣₜᵢₐₗ(\dot{x})+W_{accelerative}(\ddot{x})

Thus, there are 3 principle forms of energy that can be delivered to the heart, and the capacity for energy story in any one form is almost independent of the capacity of storage in the other forms. Thus to optimally and indeed naturally supply energy to the circulatory system, one must supply that energy is all these different forms. The proportion of the energy stored in these forms depends on the pressure in the Cup, the distance the liner displaces the heart free walls, the first time derivative of that displacement (slope) and the second time derivative of that displacement (rate of slope change).

In most discussions of extra-cardiac assist devices and all disclosures of such devices have treated the energy contribution to the heart as purely baric, i.e., the rate and location of fluid entering the device is not discussed. Performing the integrations above, we have calculated the contributions of each of the energy components for normal cardiovascular parameters supported by an extra-cardiac assist device in one cardiac cycle at 90 beats/min. On the left is the contributions with high velocity directed flow (present invention), and on the right the contributions in a low velocity uniform pressure inflation mode (extra-cardiac assist).

### Physiologic Extra-Cardiac Assist Device

W_{baric}=0.0050 Joules W_{baric}=0.0140 Joules
Wᵢₙₑᵣₜᵢₐₗ=0.0025 Joules Wᵢₙₑᵣₜᵢₐₗ<0.0005 Joules
W_{accelerative}=0.0025 Joules W_{accelerative}<0.0005 Joules

The power per beat contributed from these forms of energy is 0.015 Joules. The total power can be calculated from an average 5 L/min (90 beats/min.) to be 1.35 Watts.
Wₚₒₜₑₙₜᵢₐₗ=0.0100 Joules Wₚₒₜₑₙₜᵢₐₗ=0.0050 Joules

About 50% of the power is stored as potential energy (kx), this is delivered by the tension in the heart wall (atrio-ventricular septum) and compliance of the aorta. We call the baric support time independent since it depends only on pressure and volume. The inertial and accelerative components of support are time dependent.

Accordingly, the velocity and acceleration of the drive fluid is critical. The velocity and acceleration of drive fluid conveys this form of energy to the liner locally. The momentum of the gas is deposited almost entirely near the point of impact, such that if the fluid flow is directed perpendicular to the liner it imparts inertial energy primarily on the first collision and a secondarily on subsequent collision located in the vicinity of the first collision. Thus a fluid jet directed at the top 25% of the left ventricle imparts nearly all of its inertial energy into the maximum blood volume of the left ventricle. The same occurs with the accelerative component, which is imparted as a non-isotropic, left-only directed dynamic force.

These two components are 25% of the total energy required. Furthermore, the difference between the potential energy in the physiologic extra-cardiac assist device case and the EAC case is another 0.005 J. This is due to the faster speed of the blood exiting the left ventricle, which due to compliance of the aorta tends to cause dilation of the aorta rather than increased blood flow. Thus potential energy and reserve blood volume is returned to the left circulation after the aortic valve closes.

In mock loop experiments where the time dependent profile of the blood flow and pressure is compared under baric and inertia conditions, the inertial case duplicates the velocity and pressure distributions found in normal cardiovascular systems, where the baric driven velocity profile was less pulsatile, and was comparable to dilated hearts even though the mean flows achieved were the same. Thus three sources of localized (left only) energy that sum to about ½ the total energy requirement for left and right circulation. In addition, left and right sides share equally the baric contribution. Consequently, up to ¾ of the total energy supplied to the heart can be directed to the left ventricular circulation. Of course an inertial system can be made baric by slowing down the rate of fluid injection and directing the flow more apically.

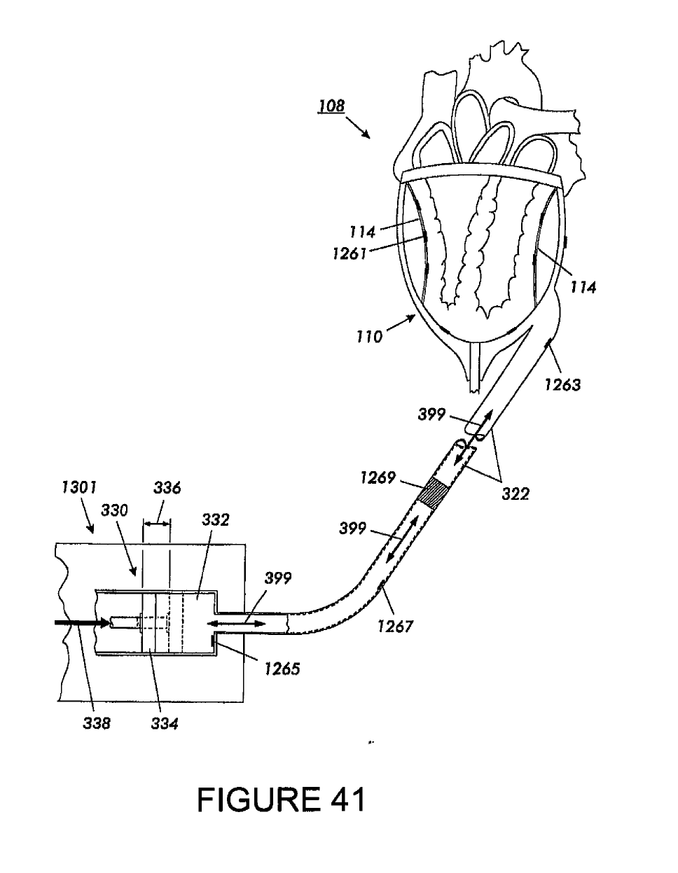

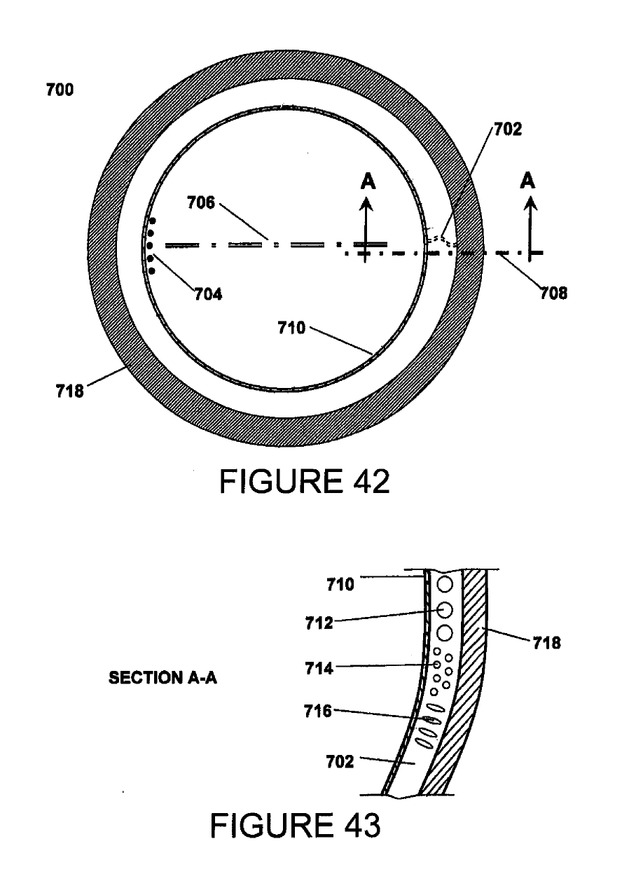

FIG. 42 is coronal view schematic of the physiologic extra-cardiac assist device Cup 700 showing elements required to achieve inertial mode heart actuation. Delaying application of baric pressure from LV to RV is achieved by partially blocking flow of drive fluid. In addition other means are used to eliminate any lateral movement of the heart within the Cup that could otherwise cause tissue damage.

Referring to FIG. 42 physiologic extra-cardiac assist device Cup 700 is shown with two different features that may be used alone or in combination. The first feature is a strip 704 of bio-adhesive that is aligned vertically in the Cup at the septal plane 706. Adhesive strip 704 serves to fix the heart so that it will not shift laterally in the Cup 700 due to an imbalance of applied forces. The second feature is a barrier membrane 702 that joins the Cup shell 718 and liner 710, and is aligned vertically at the septal plane 706.

In FIG. 42 the barrier membrane 702 is shown as being un-stretched or slack; alternatively it may be designed to be under tension at full diastole, thus providing elastic rebound energy during the diastolic portion of Cup operation.

Section view FIG. 43, taken at section A-A 708 from FIG. 42, shows more detail of the barrier membrane 702 and the perforations that permit drive fluid flow from the initial entry location on the LV side of the Cup toward the RV side. Shell 718 is connected to liner 710 at a location proximate to the septal plane 706 as shown in FIG. 42. The size, number, and location of perforations will be chosen to provide the appropriate timing and flow of drive fluid; three alternatives are shown schematically in FIG. 43.

One approach uses a small number of relatively large perforations 712.

A second approach is to use a larger number of smaller perforations 714; this will cause a larger relative timing offset between pressurization of LV and RV portions of the Cup due to the non-linear relationship of pressure drop and flow to the size of an orifice.

A third alternative using oval perforations 716 may be used to minimize the effects of material fatigue during cycling over a large number of Cup actuations. The three alternatives suggested by perforations 712, 714, and 716 may be used individually or in combination to optimize Cup performance and reliability.

It will be appreciated by those skilled in the art that the use of a bio-adhesive strip 704 and/or a barrier membrane 702 can be effective in preventing heart volley and septal bulging, both of would otherwise degrade the performance of the Cup and potentially cause trauma to the heart.

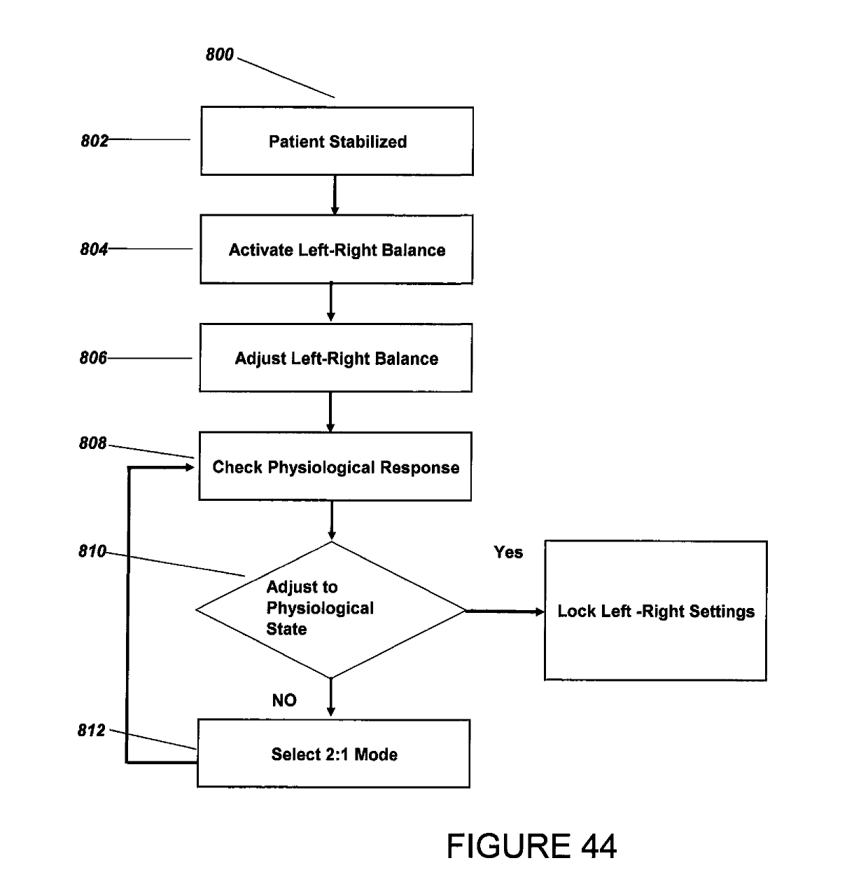

FIG. 44 is an algorithm 800 which can be employed additionally to the algorithms already mentioned that principally controls the rate of Cup inflation to change the balance of flow between the left and right sides.

In step 802, the clinician has determined that the patient has reached some form of cardiovascular steady state, such that changes in left-right output would provide reliable convergence to a more preferred support regimen. Auxiliary clinical parameters the clinician may check are arterial pressure pulsatility, arterial blood flow, or a heart imaging means, such as ultrasound.

However, in general additional surveillance strategies are not required, since increases in left heart output are almost immediately apparent in terms of lowering of venous pressure, improvement in pulmonary function and improvement in blood oxygenation. The primary piece of information a clinician needs to know to chart a path to improve support is whether the change in drive parameters increases or decreases left ventricular stroke volume relative to right ventricular stroke volume.

A variety of approaches may be taken to provide a graphic user interface that clearly represents proper left-right output and departures from same, for purposes of system adjustment.

In step 804 the clinician activates this mode of operation and step 806 adjusts the left-right balance in a preferred direction.

In step 808 the clinician rechecks his physiological parameters and confirms expected improvement. In step 810, the clinician continues to adjust until a balanced state or another preferred cardiovascular state is reached. In check 810, the flow balance controls are locked in their current settings.

If a desired state has not been reached the clinician continues to step 812 where a 1:2 actuation mode is selected. The system will begin actuating the cup every other beat if the synchronous mode is in use, or the system will halve the console input beat frequency. The clinician may alternatively elect to change any other console setting such as % systole. In general, greater duration of systolic actuation will proportionally increase the inertial to baric energy ratio. Then the algorithm returns to step 808, physiological parameters are checked, and step 810 balance control is adjusted, etc.

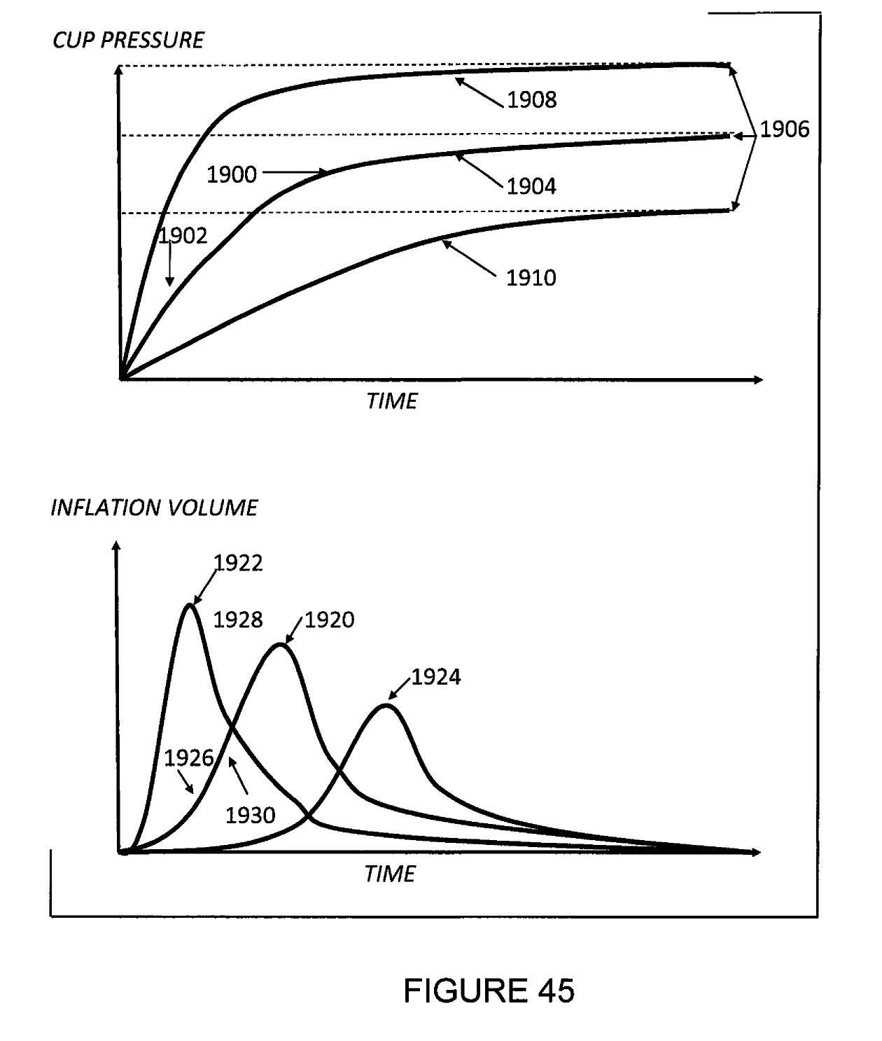

The action of the balance control is to increase or decrease the pressure of the positive pressure reservoir. FIG. 45 illustrates typical velocity and pressure course as measured in the cup at the opening of the servo valve of the pressure reservoir. The typical pressure profile 1900 is a steeply rising initial interval 1902 followed by an asymptotic plateau 1904 converging to the target reservoir pressure 1906. Curve 1908 corresponds to a higher reservoir pressure and curve 1910 corresponds to a lower reservoir pressure.

The corresponding velocity profiles are 1920, a higher reservoir pressure 1922, and a lower reservoir pressure 1924. The inertial part of the curve is section 1926. Point 1928 indicates the peak velocity and maximum inertial point and Point 1930 indicates the peak acceleration and maximum accelerative point.

The inertial and accelerative energy components scale linearly with pressure in the pressure range under consideration, which is typically 100-150 mmHg. However, their relative % to the total support energy is not linear.

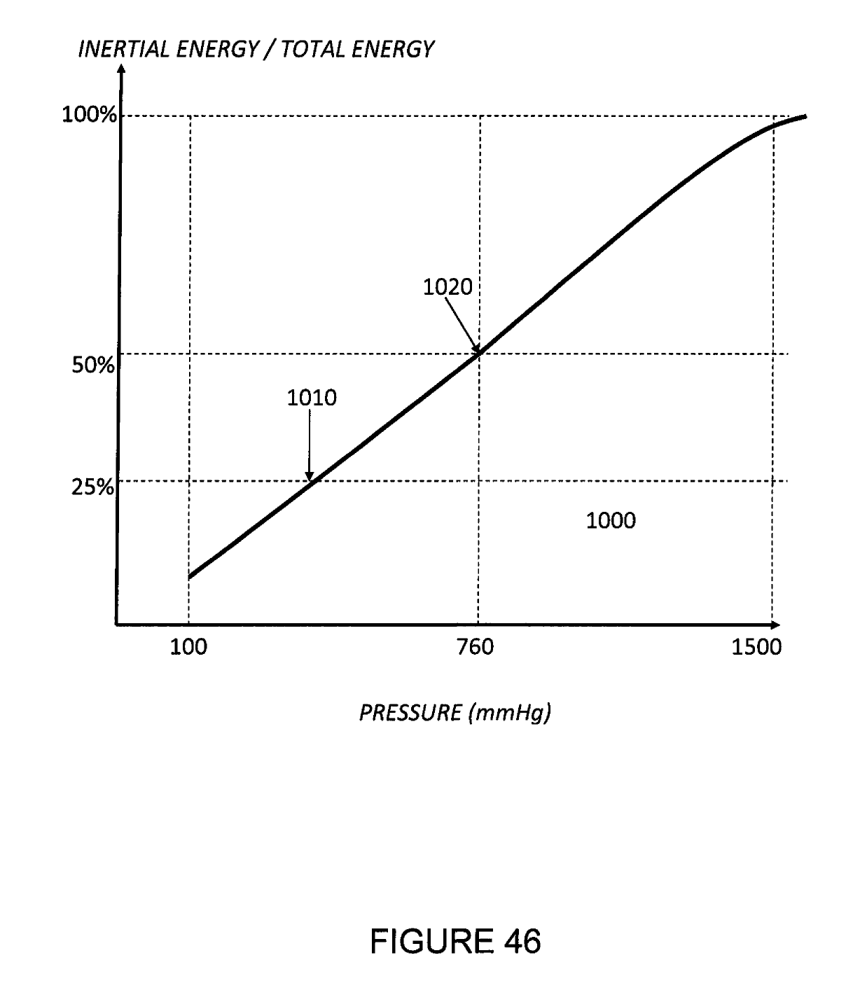

FIG. 46 shows the % inertial energy of total support energy 1000 for 72 actuations per minute for pressure 100 mmHg to 1500 mmHg. This plot is provided for illustrative purposes only, but the relative scaling is similar for all system configurations. The magnitude of any particular curve depends on the orientation of the inflow port, the geometry of the left-right partition, the diameter of the drive tube, the capacity of the pressure reservoir vessel, the rate of refill of the pressure reserve vessel, as well as other factors known in the art.

These calculations assume the inflow port is located on the LV side of the Cup. Point 1010 indicates approximately location of left-right balance for normal sized healthy hearts depressed using Esmolol, and 1020 indicates approximate location of left-right balance for congested larger than normal hearts depressed using LAD (left anterior descending artery) ligation.

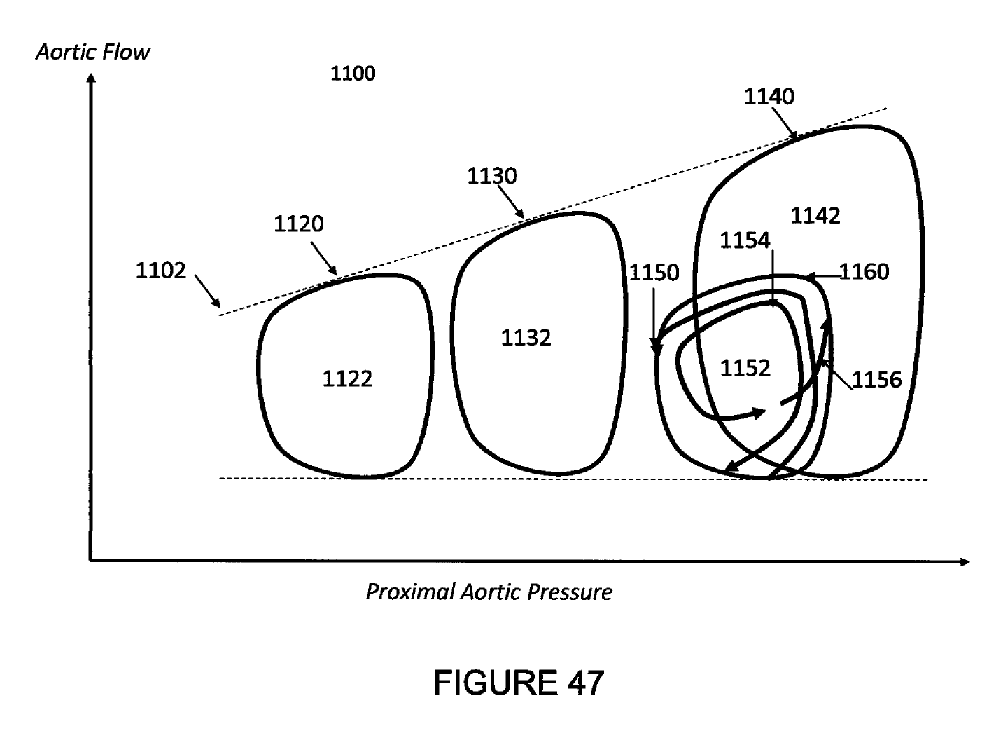

FIG. 47 illustrates a clinically useful form of displaying the effects of left-right balance and assessing the Frank-Starling mechanism in a patient on physiologic extra-cardiac assist device support. To understand this method of assessing clinical status we will present a physical interpretation of what we call the (P,F)-loop representation of hemodynamics. This representation is portrayed from the perspective of either the left or right side, where typically the left side perspective is chosen.

In this case P is the left ventricular or aortic pressure and F is the aortic flow. This representation is preferred over the typical pressure-volume representation since left ventricular volume is difficult to obtain. We wish to show that the area inscribed by the (P,F)-loop is proportional to the amount of energy produced by the device-heart system. In the case when the device is operated in 1:2 mode it is possible to quantify the degree of dependence of the native heart on the device by comparing the area of the loop when the system is OFF and the area of the loop when the system is ON.

First, the Steady Flow/Pressure Case (P=constant, F=constant). Ignoring potential energy terms due to gravity, compliance and compressibility the unit energy of a fluid system is E=ρVv² where ρ is the mass density, V is the unit volume, v is the speed, assumed to be constant everywhere in the unit volume V.

The work per cycle is the area enclosed by the (P,F)-loop. A (P,F)-loop that is a point or a line segment describes a pump that does no work, and for all (P,F)-loops, the area enclosed by the (P,F)-loop is the work per cycle. The area enclosed in a (P,F)-loop has units Energy/Time, and assuming every loop takes the same time to complete, the area equals the energy per cardiac cycle (times a scaling factor) delivered to the circuit from the pump.

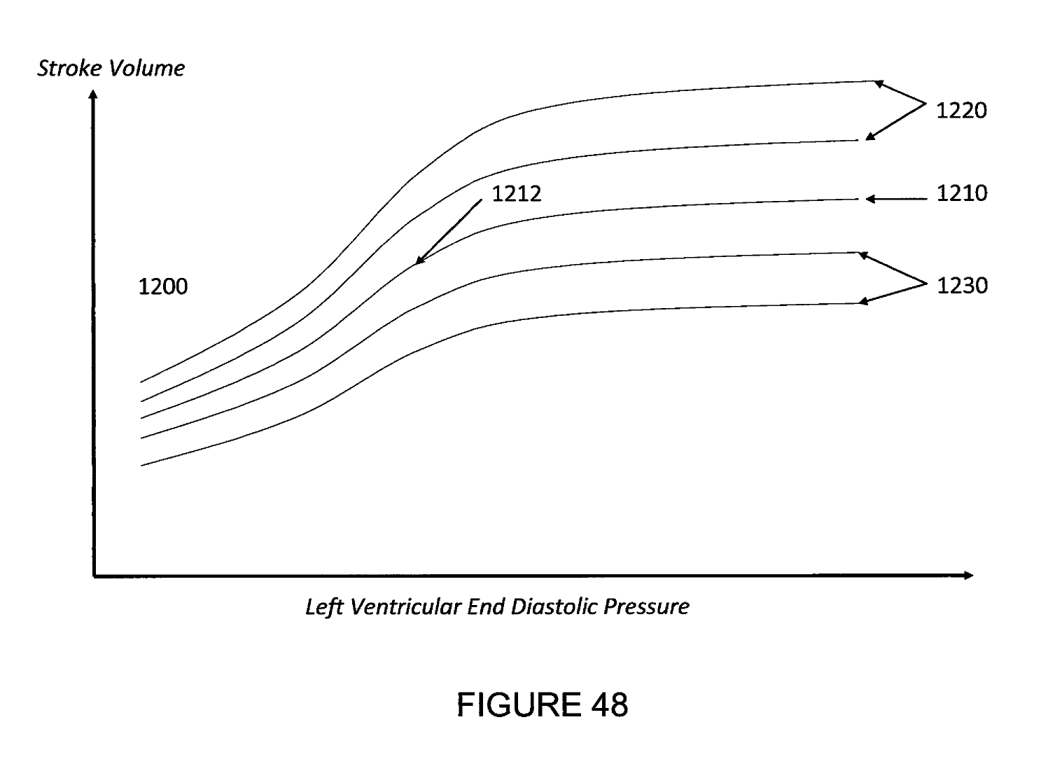

Referring to FIG. 47, a typical (P,F)-plot 1100 of a heart passing successively through four states indicated as 1120 1130, 1140, 1150. The areas 1122, 1132, 1142, and 1152 are proportional to the amount of work performed during a cardiac cycle. The sequence 1140 to 1130 to 1120 is typical for a heart under the action of a short lived beta blocker administered intravenously in two successive doses (1130 and 1120). The curve 1102 (dashed line) is the (P,F) representation of the Frank-Starling mechanism, normally represented in phase space (P,V) as illustrated in FIG. 48.

Such a curve is indicative of a healthy accommodative response to a change in cardiovascular state. Each of these curves 1140, 1130, 1120 represents an equilibrium state. These are the states we would target a supported heart to move through on the path to recovery. The sequence 1140 to 1150 is a typical plot for a heart in which the aorta is partially occluded. This mimics a condition in which the left and right sides of the heart are pure barically driven such that the right output is approximately 1.2× the left output.

Note the spiral feature 1154; this is the long term time progression of curve 1150, typically this sort of progression end in heart fibrillation or asystole. In this time progression the arterial pressure has become much less pulsatile and the mean pressure ½ or less of the normal value. In this case, the left and right ventricular pressures are not so different, and the transition indicated by 1156 occurs and the heart is supported out to curve 1160. Neither curve 1150 or 1160 lie on the Frank-Starling curve 1102. Consequently, these states are not in cardiovascular equilibrium, and the heart is entirely dependent upon the external support.

Referring to FIG. 48, 1200 is a plot of the Frank-Starling mechanism. Curve 1210 is for a normal heart showing that return to the left ventricle increases left ventricular end diastolic pressure (LVEDP) and volume, thereby increasing ventricular preload. This results in an increase in stroke volume (SV). The normal operating point 1212 is at a LVEDP of 8 mmHg and a SV of 70 ml/beat. The Frank-Starling mechanism is fully represented by a family of curve 1220 and 1230. Changes in after load and inotropy shift the Frank-Starling curve up 1220 or down 1230. However, these are normal equilibrated responses to changes in the cardiovascular state.

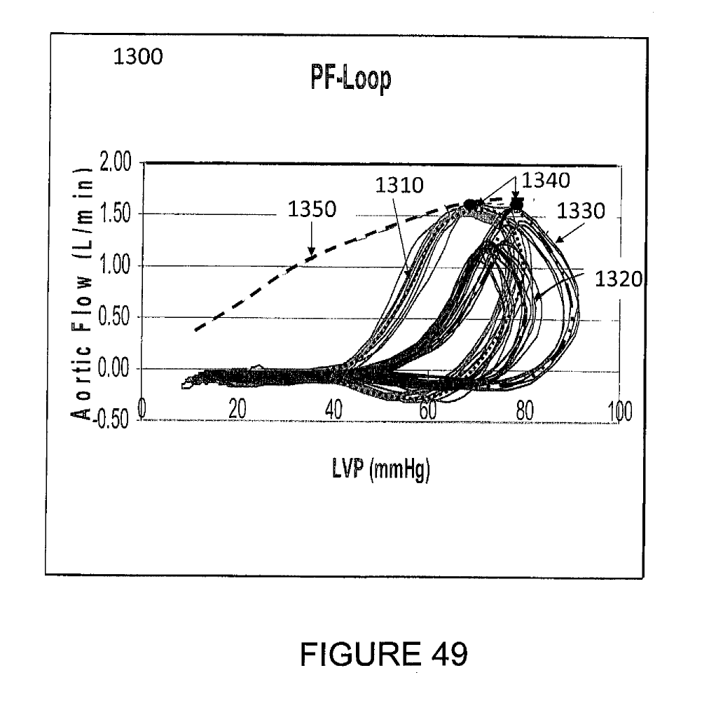

Referring to FIG. 49, plot 1300 is an actual trace of rabbit (P, F)-loops for baseline 1310, Esmolol depressed heart under pure baric support 1320, and Esmolol depressed heart under pure baric support with pulmonary artery flow (PAF) occluded to 50% of baseline 1330. The increased after load improved left side output and work (area) and returned the (P,F)-loop to the Frank-Starling curve 1350 (dashed line) and 1340 (point).

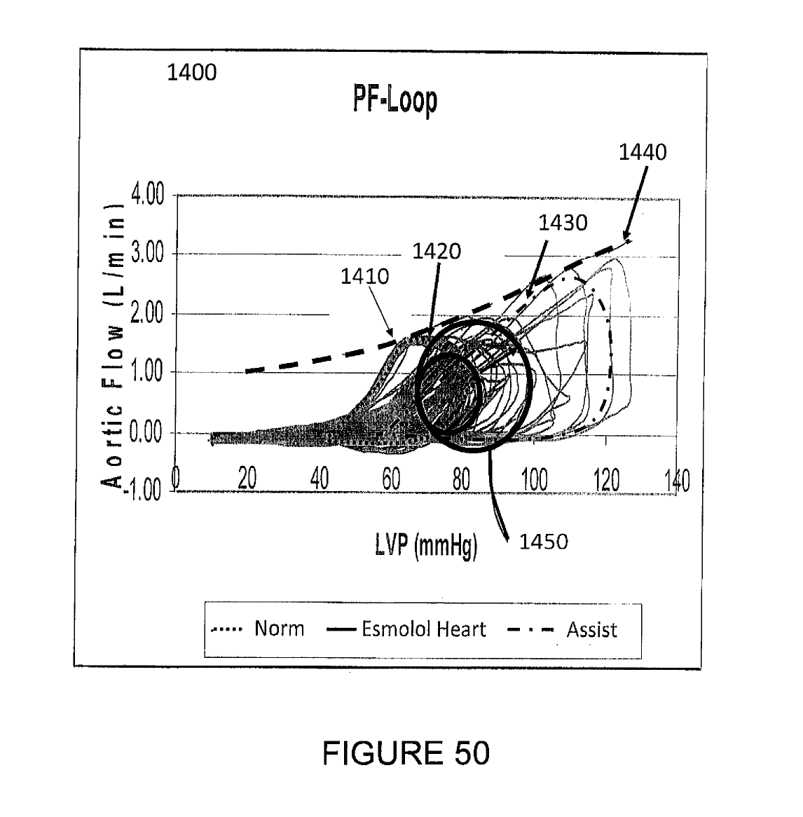

Referring to FIG. 50, a plot of normal rabbit heart 1410, Esmolol depressed heart 1420, and physiologic extra-cardiac assist device assisted heart 1430 is shown. Curve 1430 shows a continuous time sequence as the inertial component of the support is increased, at levels where the inertial/baric ratio is insufficient for left-right balance 1450 the support (P,F)-loop is below the Frank-Starling curve (dashed, 1440) the other curves where the inertial/baric ratio provides left-right balance the loops trace out the Frank-Starling curve (dashed, 1440).

A particular physiologic extra-cardiac assist device system is now described exemplifying some of the simplifying features over prior art extra-cardiac assist systems and a suitable user interface.

The physiologic extra-cardiac assist device system includes an implantable ventricular assist device (Cup) designed to support left and right ventricular function of the heart when the natural heart, with the aid of standard drug therapy and/or intra-aortic balloon counterpulsation, is unable to maintain normal blood flow and pressure in the vascular beds.

Circulatory support is achieved by unloading the right and left ventricles of the heart by periodically applying external forms of energy to the heart free walls to eject equal volumes of blood from the left and right ventricles at near normal levels and to provide pulsatile blood flow at normal arterial pressures. The physiologic extra-cardiac assist device Cup is stabilized on the heart by a combination of apical suction, a size-adjustable base seal, internal hydrophilic strips and optional liner that both become tacky in the presence of body fluids.

The combination of size-adjustable base seal and optional liner allow a single size physiologic extra-cardiac assist device Cup to support most patients, one over-sized cup size is needed for chronically enlarged hearts. In general, this particular embodiment of the physiologic extra-cardiac assist device system is designed for emergency use and should not require any special preparation such as sizing the Cup to the heart. The physiologic extra-cardiac assist device Cup is provided with integrated drivelines for connection to physiologic extra-cardiac assist device Consoles. The physiologic extra-cardiac assist device Cup is capable of providing circulatory support with the chest open or closed. The physiologic extra-cardiac assist device Cup base seal allows one cup size to be effective in supporting a variety of heart sizes. The physiologic extra-cardiac assist device Cup is provided sterile and intended as a single-use device.

The physiologic extra-cardiac assist device Console actuates the Cup using compressed room air. The system normalizes the pressure of the air in the drivelines by periodically venting to the atmosphere before and during circulatory support. The Console applies a slight vacuum to the actuation volume (liner cavity) of the Cup during diastole to facilitate ventricular filling.

In addition, there is a slight vacuum applied to the apex of the heart to fix the Cup on the heart during circulatory support.

The physiologic extra-cardiac assist device Cups will be provided with the following accessories: base seal inflation device, implantation kit, two external driveline extensions.

The system will have the following intended uses and indications.

The physiologic extra-cardiac assist device Ventricular Assist System is intended to support and potentially recover the failing heart and provide temporary support for patients suffering from acute heart failure until a longer term treatment has been prescribed or the patient recovers. In particular, the physiologic extra-cardiac assist device Ventricular Assist System is indicated for use in patients with any cardiac disorder resulting in ventricular dysfunction in which use of the device is likely to allow native heart recovery. This includes, but is not limited to: cardiogenic shock, failed cardiac transplant, low output syndrome, acute cardiac disorders leading to hemodynamic instability, and viral myocarditis.

System Alarms, Alerts, and Status/Prompts are primarily features of the physiologic extra-cardiac assist device console. The physiologic extra-cardiac assist device Console provides the medical professional with comprehensive HELP SCREENS for all the Alarms and Alerts. In the presence of an Alarm or Alert message, the medical professional can utilize the HELP SCREEN by pressing the HELP key on the monitor keyboard. The medical professional will be provided with step-by-step troubleshooting instructions. Detailed information on all Alarms, Alerts, and Status/Prompts will be provided in a physiologic extra-cardiac assist device Console Operating Manual.

Alarm messages are displayed in the Alarm Messages section of the monitor display. Direct myocardial actuation will continue and a steady tone will sound. Systole Alarms are alarms that indicate that the heart is generating contractility associated with an erratically varying Cup Volume waveform. Pumping will continue, checking patient status is urgently needed. The following are some typical messages:

| MESSAGE | CAUSE |
|---|---|
| Cup Detached | Erratic vacuum pressure waveform. |
| | Cup is intermittently on and off the |
| | heart. |
| Vacuum Lost | Pressure is too high in the vacuum line; |
| | it may be detached from the Console, or |
| | its extension. |
| Cup Rupture | High flow, leakage of actuation volume |
| | into apical driveline |
| Systolic Interference | The Cup Volume waveform is varying |
| | with a regular period |

System Surveillance Alarms are provided by the physiologic extra-cardiac assist device Console constituting internal surveillance of all key drive unit functions and of drive parameters entered by the medical professional and stored within the console.

| MESSAGE | CAUSE |
|---|---|
| Electrical Test Fails | Electrical failure during power-up |
| diagnostics | |
| Processor Failure | Microprocessor or other |
| | electronic/pneumatic failure |
| Compressor Failure | Compressor fails to develop |
| | pressure or vacuum reservoir |
| | pressure |
| Vacuum Failure | Vacuum pump supplying the |
| | apical driveline fails to develop |
| | sufficient vacuum pressure |

Alert messages are displayed in the ADVISORY section of the screen. Patient status is not immediately in jeopardy. A double beep tone for 30 seconds signals the medical professional that corrective action is needed. The Alert message remains displayed until the alert condition is corrected.

| MESSAGE | CAUSE |
|---|---|
| Pressure Leak | A leak in the liner actuation system |
| Vacuum Leak | A leak in the apical suction system is causing |
| | a high net flow, but vacuum pressure is |
| | maintained |
| Low Battery | Battery time is below 30 minutes of |
| | operating time |
| Prolonged Time in Standby | System has been in standby for at least 20 |
| | minutes |
| Maintenance Required | System maintenance may be required |
| Code # | |
| No Patient Status Available | External signals were not connected or failed |

Status/Prompt messages are displayed in the ADVISORY section of the display. Status/Prompt messages do not sound any tones and are advisory in nature.

| MESSAGE | CAUSE |
|---|---|
| System Test OK | Power-up diagnostics tests passed |
| Ready to INITIATE | Notifies the medical professional the |
| Battery in Use | Internal battery is being used |

The user sets duration by specifying heart rate and % systole. The user can decrease the effective Cup volume by increasing the end diastolic pressure. The default setting for end diastolic pressure is zero (0). Actuation vacuum pressure is adjusted automatically in response to changes in circulatory volume or preload. Actuation vacuum pressure increases automatically with increased preload.

The user sets duration by specifying heart rate and % systole. The user sets stroke volume by specifying end systolic pressure. Actuation pressure is automatically adjusted in response to changes in after load. Actuation pressure automatically increases with increased after load.

The physiologic extra-cardiac assist device system can be run in an asynchronous mode where Cup actuation is not synchronized with native heart function. Generally, a heart that is recovering some of its systolic function will begin contracting in synchrony with the mechanical actuation of the heart. However, if the heart begins contracting during the diastolic phase of cup actuation it will tend to resist the motion of the Cup bladder, this can be detected in the pressure line supplying cup actuation.

Symptoms of interference between asynchronous operation and native heart contractions are modulation of the peak cardiac output across several actuation cycles; decreasing cardiac output; increased filling pressures; and/or pulmonary edema.

Adjustment of system parameters should be undertaken to achieve better alignment of the native heart function with the actuation of the physiologic extra-cardiac assist device Cup. These actions may include switch to synchronous operation, changing system actuation rate or increasing Cup suction.

Systolic volume is the sum of left and right stroke volumes. There is no simple way to directly detect an imbalance in left/right stroke volumes. Flow imbalance is most commonly manifested as elevated wedge pressure or pulmonary edema. Flow balance is improved by initiating the flow balance mode.

FIG. 47 details the steps taken while operating in flow balance mode. Results while in flow balance mode can be enhanced by increasing systolic pressure; increasing Systolic duration (% Systole); reducing actuation rate; and/or increasing the suction on the apical vacuum line (reducing pressure).

If an external ECG line is connected to the physiologic extra-cardiac assist device Console the signal will be displayed on the monitor in red. The physiologic extra-cardiac assist device Console will display a ready message on the console screen when synchronous operation may be initiated.

If an external pressure line is connected to the physiologic extra-cardiac assist device Console the signal will be displayed on the monitor in red. The physiologic extra-cardiac assist device Console does not use this signal to adjust pump pressures; this must be done manually using the SYSTOLIC PRESSURE and DIASTOLIC PRESSURE keys. The physiologic extra-cardiac assist device Console will select an initial reservoir pressure based on the keyed in cup size. This setting is overwritten by instructions entered using the SYSTOLIC PRESSURE key.

The internal signal generator creates asynchronous direct myocardial actuation. The system begins at the selected heart rate at ½ the selected systolic pressure, or ½ the default systolic pressure for the selected cup. The system slowly increases the pressure to the target or selected pressure. The pressure control is locked out during this phase, and is indicated by flashing of the SYSTOLIC PRESSURE key. The following are control panel setting options.

TABLE 1
Control Keys and Operational Ranges

| Label | Range | Increments |
|---|---|---|
| CUP SIZE | 80-140 mm | 5 mm/10 mm |
| SYSTOLIC PRESSURE | 90-300 mmHg | 10 mmHg |
| DIASTOLIC PRESSURE | 0-−100 mmHg | 10 mmHg |
| % SYSTOLE | 35%-60% | 1% |
| | (250 ms min.) | |
| HEART RATE | 60-120 BPM | 5 BPM |
| | asynchronous | |
| APICAL SUCTION | 0-−100 mmHg | 10 mmHg |

The physiologic extra-cardiac assist device system has been designed for rapid circulatory support. The timing of the implantation can be critically important to the clinical outcome. This section describes the procedures for implanting the physiologic extra-cardiac assist device Cup and initiating biventricular support. This procedure can be applied to Cup implantation through a sternotomy or left thoracotomy. The Cup is flexible enough to be inserted through a minimal-incision thoracotomy.
1. Expose the heart and open the pericardium from the apex to just above the left atrial appendage. Avoid damaging the phrenic nerve. Adhesions between the ventricles and pericardium should be taken down to allow trauma-free placement of the physiologic extra-cardiac assist device Cup around the heart within the pericardial cavity.
2. Establish console power, verify MAINS POWER SWITCH and CONSOLE ON/OFF SWITCH are ON.
3. Connect all external data streams to the console.
4. Verify the data displayed on the Console monitor is equivalent to the data displayed on external monitors.
5. Instruct the perfusionist to allow volume into the heart if on CPB.
6. (IF a sterile procedure is to be used) Examine the two External Driveline Extensions. One side of each driveline will be capped; the other side will be open. Pass the open ends of the drivelines out of the sterile field. Secure the sterile, capped portions of the driveline extensions to the sterile drape.
7. Connect the Console to the non-sterile ends of the drivelines. The driveline connections at the console are of different size to avoid improper connection.
8. Connect the Cup drivelines to the sterile side of the driveline extensions. First remove the A caps and connect the apical driveline to the A driveline extension. Second remove the B caps and connect the bladder driveline to the B driveline extension. These connections are of different size to avoid improper connection.
9. Connect the Cup drivelines to the sterile side of the driveline extensions. First remove the A caps and connect the apical driveline to the A driveline extension. Second remove the B caps and connect the bladder driveline to the B driveline extension. These connections are of different size to avoid improper connection.
10. At the Console, push the INITIATE key on the Console. Check that suction is supplied to the apex of the cup and the actuation bladder is in the contracted, diastolic position (against the Cup shell).
11. Place the physiologic extra-cardiac assist device Cup on the heart and use the Rim Seal inflation kit to fit the Cup to the heart.
12. At the Console, select the system parameters: actuation rate (AR), systolic pressure (SP), systolic duration (% SYSTOLE), and diastolic pressure (DP).
13. Push the INITIATE key again. The cup will begin to actuate. The INITIATE key will cease to flash. Examine the expansion and contraction of the actuation bladder.
14. Monitor the apex driveline for blood. If blood is seen in the driveline, consideration should be given for the possibility of seal leakage.

Volume administration, vasoactive pharmacologic support, and efficient removal of fluid overload are important factors in successful management. Optimization of hemodynamic parameters is key in achieving maximum cardiac output and systemic perfusion. To properly assess right- and left-sided pressure values, a Swan-Ganz® catheter with continuous oximetry is recommended for the duration of support on the physiologic extra-cardiac assist device system.

TABLE 2
Recommended Target Hemodynamics
Recommended Target Hemodynamics

| Parameter | Target Value |
|---|---|
| MAP | 70-80 mmHg |
| CVP | 12-16 mmHg |
| LA/PCWP | 12-14 mmHg |
| SVR | Approx. 1000 dyne × sec/cm⁵ |
| PVR | <250 dynes × sec/cm⁵ |
| CI | >2.2 L/min./m² |

It is, therefore, apparent that there has been provided, in accordance with the present invention, a method and apparatus for Physiological Extra-Cardiac Actuation. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A device for restoring cardiovascular equilibrium comprising:
a cup configured to encompass, and to seal to the atrio-ventricular groove of a heart;
said cup applying inertial and baric energy to the left ventricle and baric energy to right ventricle;
said cup localizing a heart such that motion of the heart's center of mass is minimized.

2. The device of claim 1, wherein the ratio of inertial energy to baric energy delivered to the heart is controllable from 4 to 0.1.

3. The device of claim 1, wherein the quotient of energy delivered to the left ventricle divided by energy delivered to the right ventricle is from 4 to 1.

4. The device of claim 1, wherein the heart localization is achieved by at least one of the following: an adjustable rim seal, hydrophilic anterior and posterior axially aligned strips positioned in the plane of the ventricular septum, or a hydrophilic circumferential apical strip encircling a portion of the heart apex.

5. The device of claim 4, further comprising:
hydrophilic strips which when exposed to body fluid become tacky and promote proteinaceous bonding between strips and epicardium.

6. The device of claim 1, wherein the actuation fluid is a gas, and the inertial component is derived by directing the flow of gas substantially perpendicular to the proximal 25% of the left ventricular free wall.

7. The device of claim 1, wherein the energy is delivered to the ventricles of a heart by delivery of an actuation fluid to a single chamber actuation volume that encircles at least 50% of the ventricular fee walls.

8. The device of claim 7, wherein the single chamber actuation volume contains two fluid flow limiting partitions running axially and located posteriorly and anteriorly and in the plane of the ventricular septum.

9. The device of claim 1, wherein the flow of blood forced out of the left and right ventricles of a heart by the device are approximately of equal volume during a cardiac cycle.

10. The device of claim 1, wherein a control means is provided that varies the ratio of inertial to baric energy supplied to the left ventricle by varying the magnitude of the first and second time derivatives of fluidic flow.

11. The device of claim 10, wherein the energy supporting actuation of the right ventricle is substantially baric energy.

12. The device of claim 11, wherein a flow liming partition delays delivery of baric energy to the right ventricle relative to delivery of baric energy to the left ventricle.

13. The device of claim 1, wherein the inertial energy delivered to the left ventricle is approximately equal to the baric energy delivered to the left ventricle and the energy delivered to the right ventricle is equal to the baric energy delivered to the left ventricle.

14. The device of claim 13, wherein the baric energy delivered to the right ventricle is delayed relative to delivery of baric energy to the left ventricle.

15. The device of claim 1, wherein the interior dimension of the cup configured to enclose the heart is approximately equal to the length of the exterior apical to base dimension of the heart and the cup has a tapered internal circular cross section approximately 25% greater in diameter than the corresponding exterior diameter of the heart.

16. The device of claim 15, wherein a rim seal located at the base of the cup configured to enclose the heart is inflatable to seal the base of the heart to the rim of the cup.

17. The device of claim 16, wherein the rim seal is an inflatable torus with outer periphery bonded hermetically to the inner surface of the cup.

18. The device of claim 17, wherein the rim seal is coated on the surface in contact with the heart with a hydrophilic and tacky coating.

19. The device of claim 18, wherein the coating is at least 1 mm thick, compliant and swells several times in thickness when exposed to body fluids.

20. The device of claim 19, wherein the action of swelling promotes bonding of proteins present in bodily fluids such that the coating bonds to the heart.

21. The device of claim 1, wherein a sheath comprised of a hydrophilic bioabsorbable material is inserted into the cup configured to enclose the heart.

22. The device of claim 21, wherein a variety of free liners can be exchangeably fitted to the interior of the cup configured to enclose the heart and improve fit between the heart and cup.

23. The device of claim 22, wherein the liner is comprised of closed cell pores and has a modulus less than 25 Shore A.

24. The device of claim 22, wherein liner is comprised of open pores designed to draw body fluid into the liner and conduct vacuum across the surface of the heart such that the Poisson ratio increases to 0.5 over the period of approximately 1 hour.

25. The device of claim 1, wherein cup is localized on the heart by supplying suction at the apex of the cup.

26. The device of claim 25, wherein energy is applied to the ventricles of a heart by fluidically and cyclically filling and evacuating a chamber situated between the ventricular free walls of a heart and a semi-rigid cup structure surrounding it.

27. The device of claim 25, wherein positive pressure is supplied by a pressurized reservoir and negative pressure is supplied by an evacuated reservoir and a switching means is provided for cyclically connecting the cup to said reservoirs.

28. The device of claim 27, wherein the inertial energy component is controlled by adjusting the target pressure of the pressurized reservoir.

29. The device of claim 1, wherein the heart is supported in its pumping in both systolic and diastolic phases of the cardiac cycle.

30. A method for restoring cardiovascular equilibrium comprising:
(a) providing a cup configured to encompass, and to seal to the atrio-ventricular groove of a heart;
(b) applying, using the cup, inertial and baric energy to the left ventricle and baric energy to right ventricle; and
(c) localizing a heart such that motion of the heart's center of mass is minimized such that a state of cardiovascular equilibrium is reached.

31. The method of claim 30, wherein variation of the ratio of inertial to baric components of energy applied to the heart places the patient in a state of cardiovascular equilibrium that responds to changes in ventricular after load and preload in a manner substantially equivalent to the Frank-Starling mechanism.

32. The method of claim 30, wherein the ratio of inertial to baric energy applied to the heart is controlled by selecting a target pressure for a pressure supply reservoir.

33. The method of claim 30, wherein the ratio of inertial to baric energy applied to the heart is controlled by regulating the first time derivative of fluid flow supplied to the cup.

34. The method of claim 30, wherein the ratio of inertial to baric energy applied to the heart is controlled by regulating the first and second time derivatives of fluid flow supplied to the cup.

35. The method of claim 30, wherein the majority of energy supplied to the right ventricle is baric and the majority of energy supplied to the left ventricle is inertial.

36. The method of claim 30, wherein at least half the energy supplied to the heart is stored proximal to the heart during the systolic phase of the cardiac cycle.

37. The method of claim 30, wherein greater than 50% of the energy supplied to the heart is supplied to the right side of the heart to support a heart in predominately right ventricular failures.

38. The method of claim 30, wherein greater than 50% of the energy supplied to the heart is supplied to the left side of the heart to support a heart in predominately left ventricular failure.

39. The method of claim 30, further comprising:
(d) weaning a heart off support supplied by an extra-cardiac assist device by
(d1) actuating the heart in synchrony with native ventricular contraction,
(d2) actuating the heart on every other cardiac cycle,
(d3) assessing the health of the heart during the non-assist cycles by measuring change in cup volume while the driveline is freely open to the atmosphere,
(d4) measuring the change in cup volume during the assist cycles,
(d5) determining a measure of heart health by comparing the changes in cup volume during assist cycles and during non-assist cycles, and
(d6) gradually reducing the amount of support energy until the heart is beating substantially on its own.

40. The method of claim 39, wherein gradually reducing the amount of support energy until the heart is beating substantially on its own commences, when the measure of heart health is less than a predetermined value.

41. The method of claim 39, wherein weaning a heart off support supplied by an extra-cardiac assist device further comprises (d7) releasing the seal around the base of the heart, (d8) making the suction line pressure slightly positive, and (d9) gradually increasing the support level until the heart decouples from the device.

42. The method of claim 41, wherein when the heart decouples from the device the liner is retained on the heart.

43. The method of claim 30, further comprising:
(d) weaning a heart off support supplied by an extra-cardiac assist device by
(d1) actuating the heart in synchrony with native ventricular contraction,
(d2) gradually reducing the amount of support energy and plotting the aortic flow as a function of aortic or ventricular pressure,
(d3) drawing a line through the end systolic points,
(d4) comparing the slope of this line to the slope of a normal heart under increasing after load conditions and
(d5) by this comparison deciding whether the heart possesses a viable Frank-Starling mechanism sufficient for terminating support.

44. The method of claim 30, wherein support level during recuperation of a heart is adjusted based on the robustness of the Frank-Starling mechanism.

45. The method of claim 30, wherein the need for continued mechanical respiratory support is alleviated by increasing the inertial component of energy supplied to a supported heart.

46. The method of claim 30, wherein a patient with abnormally high venous pressure has their venous pressure reduced by increasing the inertial component of energy supplied to a supported heart.

47. The method of claim 30, wherein poor end organ perfusion is improved by increasing the inertial component of energy supplied to a supported heart.

48. The method of claim 30, wherein donor organs are preserved longer by increasing the inertial component of energy supplied to a supported heart.

49. The method of claim 30, wherein a patient with coronary stenosis has their coronary artery perfusion improved by reducing the proportional duration of systole and maximizing the potential energy stored proximal to the heart.

50. The method of claim 30, wherein a sheath is used for patients who have recently received bypass graft surgery.

51. The method of claim 30, wherein a liner is used with an inflexible patch embedded in the otherwise flexible construction of the liner to prevent force being directly applied to a friable region of a supported heart.

52. The method of claim 30, wherein a patient is initially supported in an asynchronous mode and later supported in a synchronous mode.
